SEQUENCE LISTING (39 sequences)

SEQ ID NO: 1 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - protein, 57 aa, Homo sapiens
AIQCPDSQFECPDFSTCCVMVDGSWGCCPMPQASCCEDRVHCCPHGAFCDLVHTRCI

SEQ ID NO: 3 - protein, 56 aa, Homo sapiens
VMCPDARSRCPDGSTCCELPSGKYGCCPMPNATCCSDHLHCCPQDTVCDLIQSKCL

SEQ ID NO: 4 - protein, 54 aa, Homo sapiens
VPCDNVSSCPSSDTCCQLTSGEWGCCPIPEAVCCSDHQHCCPQGYTCVAEGQCQ

SEQ ID NO: 5 - protein, 56 aa, Homo sapiens
DVECGEGHFCHDNQTCCRDNRQGWACCPYRQGVCCADRRHCCPAGFRCAARGTKCL

SEQ ID NO: 6 - protein, 56 aa, Homo sapiens
GGPCQVDAHCSAGHSCIFTVSGTSSCCPFPEAVACGDGHHCCPRGFHCSADGRSCF

SEQ ID NO: 7 - protein, 57 aa, Homo sapiens
DVKCDMEVSCPDGYTCCRLQSGAWGCCPFTQAVCCEDHIHCCPAGFTCDTQKGTCEQ

SEQ ID NO: 8 - protein, 56 aa, Homo sapiens
DIGCDQHTSCPVGQTCCPSLGGSWACCQLPHAVCCEDRQHCCPAGYTCNVKARSCE

SEQ ID NO: 9 - DNA, 2130 bp, Homo sapiens
gctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaa

SEQ ID NO: 10 - DNA, 171 bp, Homo sapiens
gccatccagtgccccgacagccagttcgagtgccccgacttcagcacctgctgcgtgatggtggacggcagctggggctgctgccccatgccccaggccagctgctgcgaggacagggtgcactgctgcccccacggcgccttctgcgacctggtgcacaccaggtgcatc

SEQ ID NO: 11 - DNA, 168 bp, Homo sapiens
gtgatgtgccccgacgccaggagcaggtgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctg

SEQ ID NO: 12 - DNA, 162 bp, Homo sapiens
gtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccag

SEQ ID NO: 13 - DNA, 168 bp, Homo sapiens
gacgtggagtgcggcgagggccacttctgccacgacaaccagacctgctgcagggacaacaggcagggctgggcctgctgcccctacaggcagggcgtgtgctgcgccgacaggaggcactgctgccccgccggcttcaggtgcgccgccaggggcaccaagtgcctg

SEQ ID NO: 14 - DNA, 165 bp, Homo sapiens
ggcccctgccaggtggacgcccactgcagcgccggccacagctgcatcttcaccgtgagcggcaccagcagctgctgccccttccccgaggccgtggcctgcggcgacggccaccactgctgccccaggggcttccactgcagcgccgacggcaggagctgcttc

SEQ ID NO: 15 - DNA, 165 bp, Homo sapiens
gtgaagtgcgacatggaggtgagctgccccgacggctacacctgctgcaggctgcagagcggcgcctggggctgctgccccttcacccaggccgtgtgctgcgaggaccacatccactgctgccccgccggcttcacctgcgacacccagaagggcacctgcgag

SEQ ID NO: 16 - DNA, 168 bp, Homo sapiens
gacatcggctgcgaccagcacaccagctgccccgtgggccagacctgctgccccagcctgggcggcagctgggcctgctgccagctgccccacgccgtgtgctgcgaggacaggcagcactgctgccccgccggctacacctgcaacgtgaaggccaggagctgcgag

SEQ ID NO: 17 - DNA, 252 bp, Artificial Sequence
  signal+GEM
atgtggaccctggtgagctgggtggccctgaccgccggcctggtggccggccagaggagcggcaacaacagcgtgggcgccatccagtgccccgacagccagttcgagtgccccgacttcagcacctgctgcgtgatggtggacggcagctggggctgctgccccatgccccaggccagctgctgcgaggacagggtgcactgctgcccccacggcgccttctgcgacctggtgcacaccaggtgcatctaa

SEQ ID NO: 18 - DNA, 300 bp, Artificial Sequence
  signal+GEM
atgtggaccctggtgagctgggtggccctgaccgccggcctggtggccggcacccccaccggcacccaccccctggccaagaagctgcccgcccagaggaccaacagggccgtggccctgagcagcagcgtgatgtgccccgacgccaggagcaggtgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgtaa

SEQ ID NO: 19 - DNA, 297 bp, Artificial Sequence
  signal+GEM
atgtggaccctggtgagctgggtggccctgaccgccggcctggtggccggccagggcccccaccaggtgccctggatggagaaggcccccgcccacctgagcctgcccgacccccaggccctgaagagggacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagtaa

SEQ ID NO: 20 - DNA, 285 bp, Artificial Sequence
  signal+GEM
atgtggaccctggtgagctgggtggccctgaccgccggcctggtggccggcaaggaggtggtgagcgcccagcccgccaccttcctggccaggagcccccacgtgggcgtgaaggacgtggagtgcggcgagggccacttctgccacgacaaccagacctgctgcagggacaacaggcagggctgggcctgctgcccctacaggcagggcgtgtgctgcgccgacaggaggcactgctgccccgccggcttcaggtgcgccgccaggggcaccaagtgcctgtaa

SEQ ID NO: 21 - DNA, 252 bp, Artificial Sequence
  signal+GEM
atgtggaccctggtgagctgggtggccctgaccgccggcctggtggccggcaagtggcccaccaccctgagcaggcacctgggcggcccctgccaggtggacgcccactgcagcgccggccacagctgcatcttcaccgtgagcggcaccagcagctgctgccccttccccgaggccgtggcctgcggcgacggccaccactgctgccccaggggcttccactgcagcgccgacggcaggagctgcttctaa

SEQ ID NO: 22 - DNA, 279 bp, Artificial Sequence
  signal+GEM
atgtggaccctggtgagctgggtggccctgaccgccggcctggtggccggcagcaaggagaacgccaccaccgacctgctgaccaagctgcccgcccacaccgtgggcgacgtgaagtgcgacatggaggtgagctgccccgacggctacacctgctgcaggctgcagagcggcgcctggggctgctgccccttcacccaggccgtgtgctgcgaggaccacatccactgctgccccgccggcttcacctgcgacacccagaagggcacctgcgagtaa

SEQ ID NO: 23 - DNA, 291 bp, Artificial Sequence
  signal+GEM
atgtggaccctggtgagctgggtggccctgaccgccggcctggtggccggcaggggcagcgagatcgtggccggcctggagaagatgcccgccaggagggccagcctgagccaccccagggacatcggctgcgaccagcacaccagctgccccgtgggccagacctgctgccccagcctgggcggcagctgggcctgctgccagctgccccacgccgtgtgctgcgaggacaggcagcactgctgccccgccggctacacctgcaacgtgaaggccaggagctgcgagtaa

SEQ ID NO: 24 - DNA, 483 bp, Artificial Sequence
  signal+GEM
atgtggaccctggtgagctgggtggccctgaccgccggcctggtggccggccagaggagcggcaacaacagcgtgggcgccatccagtgccccgacagccagttcgagtgccccgacttcagcacctgctgcgtgatggtggacggcagctggggctgctgccccatgccccaggccagctgctgcgaggacagggtgcactgctgcccccacggcgccttctgcgacctggtgcacaccaggtgcatcaaggaggtggtgagcgcccagcccgccaccttcctggccaggagcccccacgtgggcgtgaaggacgtggagtgcggcgagggccacttctgccacgacaaccagacctgctgcagggacaacaggcagggctgggcctgctgcccctacaggcagggcgtgtgctgcgccgacaggaggcactgctgccccgccggcttcaggtgcgccgccaggggcaccaagtgcctgtaa

SEQ ID NO: 25 - DNA, 963 bp, Artificial Sequence
  signal+GEM
atgtggaccctggtgagctgggtggccctgaccgccggcctggtggccggccagaggagcggcaacaacagcgtgggcgccatccagtgccccgacagccagttcgagtgccccgacttcagcacctgctgcgtgatggtggacggcagctggggctgctgccccatgccccaggccagctgctgcgaggacagggtgcactgctgcccccacggcgccttctgcgacctggtgcacaccaggtgcatccagggcccccaccaggtgccctggatggagaaggcccccgcccacctgagcctgcccgacccccaggccctgaagagggacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagaggggcagcgagatcgtggccggcctggagaagatgcccgccaggagggccagcctgagccaccccagggacatcggctgcgaccagcacaccagctgccccgtgggccagacctgctgccccagcctgggcggcagctgggcctgctgccagctgccccacgccgtgtgctgcgaggacaggcagcactgctgccccgccggctacacctgcaacgtgaaggccaggagctgcgagaaggaggtggtgagcgcccagcccgccaccttcctggccaggagcccccacgtgggcgtgaaggacgtggagtgcggcgagggccacttctgccacgacaaccagacctgctgcagggacaacaggcagggctgggcctgctgcccctacaggcagggcgtgtgctgcgccgacaggaggcactgctgccccgccggcttcaggtgcgccgccaggggcaccaagtgcctgtaa

SEQ ID NO: 26 - DNA, 51 bp, Homo sapiens
atgtggaccctggtgagctgggtggccctgaccgccggcctggtggccggc

SEQ ID NO: 27 - protein, 83 aa, Artificial Sequence
  Signal + linker/leader + GEM F (GEM 1)
MWTLVSWVALTAGLVAGQRSGNNSVGAIQCPDSQFECPDFSTCCVMVDGSWGCCPMPQASCCEDRVHCCPHGAFCDLVHTRCI

SEQ ID NO: 28 - protein, 99 aa, Artificial Sequence
  signal+linker+GEM
MWTLVSWVALTAGLVAGTPTGTHPLAKKLPAQRTNRAVALSSSVMCPDARSRCPDGSTCCELPSGKYGCCPMPNATCCSDHLHCCPQDTVCDLIQSKCL

SEQ ID NO: 29 - protein, 98 aa, Artificial Sequence
  signal+linker+GEM
MWTLVSWVALTAGLVAGQGPHQVPWMEKAPAHLSLPDPQALKRDVPCDNVSSCPSSDTCCQLTSGEWGCCPIPEAVCCSDHQHCCPQGYTCVAEGQCQ

SEQ ID NO: 30 - protein, 94 aa, Artificial Sequence
  signal+linker+GEM
MWTLVSWVALTAGLVAGKEVVSAQPATFLARSPHVGVKDVECGEGHFCHDNQTCCRDNRQGWACCPYRQGVCCADRRHCCPAGFRCAARGTKCL

SEQ ID NO: 31 - protein, 83 aa, Artificial Sequence
  signal+linker+GEM
MWTLVSWVALTAGLVAGKWPTTLSRHLGGPCQVDAHCSAGHSCIFTVSGTSSCCPFPEAVACGDGHHCCPRGFHCSADGRSCF

SEQ ID NO: 32 - protein, 92 aa, Artificial Sequence
  signal+linker+GEM
MWTLVSWVALTAGLVAGSKENATTDLLTKLPAHTVGDVKCDMEVSCPDGYTCCRLQSGAWGCCPFTQAVCCEDHIHCCPAGFTCDTQKGTCE

SEQ ID NO: 33 - protein, 96 aa, Artificial Sequence
  signal+linker+GEM
MWTLVSWVALTAGLVAGRGSEIVAGLEKMPARRASLSHPRDIGCDQHTSCPVGQTCCPSLGGSWACCQLPHAVCCEDRQHCCPAGYTCNVKARSCE

SEQ ID NO: 34 - protein, 160 aa, Artificial Sequence
  signal+linker+GEM
MWTLVSWVALTAGLVAGQRSGNNSVGAIQCPDSQFECPDFSTCCVMVDGSWGCCPMPQASCCEDRVHCCPHGAFCDLVHTRCIKEVVSAQPATFLARSPHVGVKDVECGEGHFCHDNQTCCRDNRQGWACCPYRQGVCCADRRHCCPAGFRCAARGTKCL

SEQ ID NO: 35 - protein, 320 aa, Artificial Sequence
  signal+linker+GEM
MWTLVSWVALTAGLVAGQRSGNNSVGAIQCPDSQFECPDFSTCCVMVDGSWGCCPMPQASCCEDRVHCCPHGAFCDLVHTRCIQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCL

SEQ ID NO: 36 - protein, 71 aa, Artificial Sequence
  GEM
AMDIGCDQHTSCPVGQTCCPSLGGSWACCQLPHAVCCEDRQHCCPAGYTCNVKARSCEKLAAALEHHHHHH

SEQ ID NO: 37 - protein, 72 aa, Artificial Sequence
  GEM
AMAIQCPDSQFECPDFSTCCVMVDGSWGCCPMPQASCCEDRVHCCPHGAFCDLVHTRCIKLAAALEHHHHHH

SEQ ID NO: 38 - DNA, 6389 bp, Artificial Sequence
  vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 6932 bp, Artificial Sequence
  vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaatcgaattctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2025/0092105 A1
(43) Pub. Date: Mar. 20, 2025

(54) GRANULIN/EPITHELIN MODULES AND COMBINATIONS THEREOF TO TREAT NEURODEGENERATIVE DISEASE

(71) Applicant: ALPHA COGNITION INC., Vancouver (CA)

(72) Inventor: Denis G. KAY, Charlottetown (CA)

(21) Appl. No.: 18/562,241

(22) PCT Filed: May 20, 2022
(86) PCT No.: PCT/CA2022/050802

§ 371 Date: Nov. 17, 2023

Related U.S. Application Data
(60) Provisional application No. 63/309,118, filed on Feb. 11, 2022.
(60) Provisional application No. 63/191,255, filed on May 20, 2021.

(30) Foreign Application Priority Data
May 20, 2021 (US) 63191255
Feb. 11, 2022 (US) 63309118

(51) Int. Cl. C07K 14/475; A61K 38/18; A61P 25/28; C12N 15/861; C12N 15/864

(52) U.S. Cl. CPC C07K 14/475; A61K 38/18; A61P 25/28; C12N 15/861; C12N 15/8645; C07K 2319/02; C12N 2710/10011; C12N 2710/14011; C12N 2740/15011

(57) ABSTRACT

Methods and compositions including granulin/epithelin modules (GEMs) or combinations thereof suitable for treating neurodegenerative diseases. Also disclosed are methods of treatment of neurodegenerative diseases, such as methods of administering therapeutic recombinant GEM proteins or gene therapies for delivering recombinant GEM gene products.

The present invention is in the field of pharmaceutical and biological agents for treating brain disease. The invention relates to granulin/epithelin modules (GEMs) or combinations thereof. The invention relates to methods and compositions comprising granulin/epithelin modules (GEMs) or combinations thereof suitable for treating neurodegenerative diseases.

The invention further relates to methods of treatment of neurodegenerative diseases using granulin/epithelin modules (GEMs) or combinations thereof, such as methods of administering therapeutic recombinant GEM proteins or gene therapies for delivering recombinant GEM gene products.

In embodiments, the invention relates to a recombinant polypeptide, or combination of multiple recombinant polypeptides, comprising two to six granulin/epithelin modules (GEMs). In embodiments, the recombinant polypeptide or combination of polypeptides comprises two to six granulin/epithelin modules (GEMs), comprising GEM E, and additionally one or more of GEM F, GEM C and GEM D.

In embodiments, the invention relates to a therapeutic nucleic acid molecule or combination of multiple therapeutic nucleic acid molecules, that express two to six granulin/epithelin modules (GEMs). In embodiments, the combination of granulin/epithelin modules (GEMs) comprises two to six GEMs, comprising GEM E, and additionally one or more of GEM F, GEM C and GEM D.

### BACKGROUND

Progranulin (PGRN) is a growth factor-like protein that is involved in the regulation of multiple processes including development, wound healing, angiogenesis, growth and maintenance of neuronal cells, and inflammation. Altered PGRN expression has been shown in multiple neurodegenerative diseases, including Creutzfeldt-Jakob disease, motor neuron disease, Parkinson's disease, and Alzheimer's disease. For example, recent studies into the genetic etiology of neurodegenerative diseases have shown that heritable mutations in the PGRN gene may lead to adult-onset neurodegenerative diseases due to reduced neuronal survival.

Progranulin has been linked to various medical conditions. For example, PGRN is involved in in lung inflammation, PGRN is a known factor involved in Gaucher disease, a common lysosomal storage disease. PGRN is also involved in neuronal ceroid lipofuscinosis (NCL), also a lysosome storage disease. Frontotemporal dementia is also associated with PGRN, some FTD being caused by mutations in an allele of GRN, the gene encoding PGRN. Spinal Muscular Atrophy has also been associated with PGRN, where PGRN overexpression has been shown to reverse impaired development of primary motor neurons. PGRN also plays a role in ALS and Huntington's disease, and low PGRN appears to be a risk factor for ALS, PD, AD, Schizophrenia, and Bi-polar conditions. Low PGRN levels are also known to be associated with peripheral inflammatory conditions like arthritis and atherosclerosis.

PGRN itself is a pro-protein that can be cleaved into smaller domains called granulin/epithelin modules (GEMs), otherwise known as granulins (A-G and p). Granulins share a highly disulfide-rich, evolutionarily conserved β-sheet fold. Such characteristics are often found in highly stable proteins that can withstand heat and pH changes. Indeed, recent studies have highlighted that progranulin cleavage, and therefore, GEM production, occurs in the acidic environment of the lysosome, through the action of lysosomal cathepsins. Individual GEMs may oppose the function of the full-length protein in cell growth and inflammation. Alternatively, some evidence suggests that GEM domains also bind to and stimulate Cathepsin D (CTSD) enzymatic activity. Although multi-GEM-sized peptides have been reported in highly degenerative brain regions from Alzheimer's disease (AD) patients, the actual molecular functions of individual GEMs, multi-GEM fragments, and the full-length protein are still incompletely understood.

Currently, there is no effective cure for many neurodegenerative diseases, such as ALS, Alzheimer's disease, or Parkinson's disease. Current treatment generally involves efforts by physicians to slow progression of the symptoms and make patients more comfortable. While there are a number of drugs in development and a limited number that are FDA approved for treatment (Riluzole, for ALS; L-dopa for Parkinson's disease; cognitive enhancers, such as Aricept, for AD) these treatments only mask the progression of neurologic disease and may act to marginally prolong the lives of some patients.

Thus, there is a significant need for methods and compositions directed to treatment of neurodegenerative diseases.

### SUMMARY OF THE INVENTION

In light of the prior art, the technical problem underlying the invention was the provision of an alternative or improved agent suitable for treating neurodegenerative disease, in particular frontotemporal dementia (FTD), Parkinson's disease, Alzheimer's disease or amyotrophic lateral sclerosis. A further object of the invention was to provide combinations of GEMs with improved biological properties with respect to the aforementioned treatments.

This problem is solved by the features of the independent claims. Preferred embodiments of the present invention are provided by the dependent claims.

Aspects and embodiments of the invention are presented as follows:

In one aspect, the invention relates to a recombinant polypeptide, or combination of multiple recombinant polypeptides, comprising two to six granulin/epithelin modules (GEMs).

In one embodiment, the recombinant polypeptide comprises two to six granulin/epithelin modules (GEMs), comprising GEM E, and additionally one or more of GEM F, GEM B, GEM C and GEM D.

In one embodiment, the recombinant polypeptide comprises two to six granulin/epithelin modules (GEMs), comprising GEM E, and additionally one or more of GEM F, GEM C and GEM D.

In one embodiment, the recombinant polypeptide comprises granulin/epithelin module (GEM) E, and additionally GEM F.

In one embodiment, the recombinant polypeptide comprises granulin/epithelin module (GEM) E, and additionally GEM C.

In one embodiment, the recombinant polypeptide comprises granulin/epithelin module (GEM) E, and additionally GEM B.

In one embodiment, the recombinant polypeptide comprises granulin/epithelin module (GEM) E, and additionally GEM F and GEM D.

In one embodiment, the recombinant polypeptide comprises granulin/epithelin module (GEM) E, and additionally GEM F, GEM D and GEM C.

In one embodiment, the recombinant polypeptide comprises two to six granulin/epithelin modules (GEMs), comprising GEM F, and additionally one or more of GEM A, GEM B, GEM E, GEM C and GEM D.

In one embodiment, the recombinant polypeptide comprises two to six granulin/epithelin modules (GEMs), comprising GEM F, and additionally one or more of GEM B, GEM E, GEM A and GEM D. The combinations provided above have been shown to exhibit improved function using in vitro assessments relevant to determining therapeutic efficacy.

As shown in the examples below, treatment with GEMs A to F, optionally in combination, such as GEM E, in combination with one or more of GEM A, GEM B, GEM F, GEM C and GEM D, shows improved levels of cell survival in motor neuron-like cell lines (NSC-34) cultured with minimal serum.

To the knowledge of the inventor(s), the combinations above have not been disclosed or tested previously and represent an advantageous combinatorial approach towards treating disease associated with neuronal cell death.

The combinations can, without limitation, be derived from those investigated in the examples disclosed below. For example:

In one embodiment, the recombinant polypeptide comprises granulin/epithelin module (GEM) F.

In one embodiment, the recombinant polypeptide comprises granulin/epithelin module (GEM) B.

In one embodiment, the recombinant polypeptide comprises granulin/epithelin module (GEM) C.

In one embodiment, the recombinant polypeptide comprises granulin/epithelin module (GEM) E.

In one embodiment, the recombinant polypeptide comprises granulin/epithelin module (GEM) G.

In one embodiment, the recombinant polypeptide comprises granulin/epithelin module (GEM) A.

In one embodiment, the recombinant polypeptide comprises granulin/epithelin module (GEM) D.

In one embodiment, the recombinant polypeptide comprises granulin/epithelin module (GEM) F, and additionally GEM A.

In one embodiment, the recombinant polypeptide comprises granulin/epithelin module (GEM) F, and additionally GEM B.

In one embodiment, the recombinant polypeptide comprises granulin/epithelin module (GEM) F, and additionally GEM C.

In one embodiment, the recombinant polypeptide comprises granulin/epithelin module (GEM) F, and additionally GEM E.

In one embodiment, the recombinant polypeptide comprises granulin/epithelin module (GEM) F, and additionally GEM D.

In one embodiment, the recombinant polypeptide comprises granulin/epithelin module (GEM) B, and additionally GEM C.

In one embodiment, the recombinant polypeptide comprises granulin/epithelin module (GEM) B, and additionally GEM E.

In one embodiment, the recombinant polypeptide comprises granulin/epithelin module (GEM) C, and additionally GEM E.

In one embodiment, the recombinant polypeptide comprises granulin/epithelin module (GEM) E, and additionally GEM F and GEM B.

In one embodiment, the recombinant polypeptide comprises granulin/epithelin module (GEM) E, and additionally GEM F and GEM C.

In one embodiment, the recombinant polypeptide comprises granulin/epithelin module (GEM) E, and additionally GEM F, GEM D, GEM C and GEM B.

In one embodiment, the recombinant polypeptide comprises granulin/epithelin module (GEM) E, and additionally GEM F, with no further GEMs, or one or more further GEMs.

Surprisingly, these GEMs and GEM combinations enabled proliferation of NSC34 cells to a greater extent than full length progranulin. By identifying individual GEMs or combinations of GEMs that exhibit beneficial effects on NSC34 cells, improvements may be achieved over the full length PGRN as known in the art.

Without being bound by theory, it appears that the administration of not all GEMs in combination, as present in full length PGRN, is beneficial to NSC34 proliferation. The isolation of one or more GEMs, without using the full combination of GEMs as in full length PGRN, are beneficial when administered. The inventor(s) have therefore found that GEMs in isolation, or in particular combinations, enable improved effects over full length PGRN, indicating that some GEMs or GEM combinations may represent a “version” or “truncated form” or “minimal form” of PGRN, optimal for neuronal cell proliferation.

In some embodiments, these GEMs or GEM combinations may be selectively effective in neuronal cells, or may induce proliferation in neuronal cells, such as NSC34, but not in other cell types. The inventor(s) have therefore identified optimal GEMs or combinations of GEMs that are “neuro-supportive”, i.e., support neuronal survival and proliferation and may therefore be therapeutically beneficial in applications of reducing neuronal cell death, i.e. in treatment of the diseases disclosed herein.

In embodiments, the GEMs or GEM combinations of the invention enable a “reduced complexity” of the active molecule compared to full length PGRN. Without being bound by theory, the inventor(s) postulates that by reducing the full length PGRN molecule to a selection of GEM combinations, comprising 2-6 GEMs, potentially unnecessary or even toxic aspects of the full length PGRN molecule can be removed, thus improving the active agent compared to longer or full length PGRN drug molecules. In some embodiments, off-target effects or other toxicities may be reduced by administering a GEM combination of the invention, compared to full length PGRN, or to combinations with large numbers of GEMs. Thus, GEM combinations with 2, 3 or 4 GEMs may, in some embodiments, be preferred over more complex molecules with combinations of 5 or 6 GEMs. GEM combinations with 2 or 3 GEMs may, in some embodiments, be preferred over more complex molecules with combinations of 4, 5 or 6 GEMs.

In embodiments, a GEM combination may exhibit two GEMs, such combinations may be referred to as GEM dimers. A GEM combination may exhibit three GEMs, such combinations may be referred to as GEM trimers.

### NSC34 Cell Proliferation:

In preferred embodiments, the GEMs and GEM combinations of the invention exhibit an enhanced effect on NSC34 cell proliferation. In embodiments, these GEM combinations relate to GEMs F+E, F+B, F+C and B+C, B+E and C+E in addition to GEMs F+E+D+C. In embodiments, these GEM combinations relate to F+E+B, and F+E+C.

In embodiments, GEM combinations including GEM E, with one or more of GEM B, C or E, show improved performance over full length PGRN or in comparison to other GEMs individually or in combination. In embodiments, the GEM combinations including GEM B, with one or more of GEM C or E, show improved performance over full length PGRN or in comparison to other GEMs individually or in combination. In embodiments, the GEM combinations including GEM C, with GEM E, show improved performance over full length PGRN or in comparison to other GEMs individually or in combination.

In embodiments, GEM combinations comprising GEM dimer or trimer combinations of GEMs E, C, B and F, appear to support cell proliferation of NSC34 motor-neuron like cell line to a greater extent than treatment with full length PGRN or other GEMs alone or in combination.

### Cathepsin D Maturation:

In preferred embodiments, the GEMs and GEM combinations of the invention exhibit improved performance in a Cathepsin D Maturation assay relative to full length PGRN. In embodiments, these GEM combinations relate to GEM F with one or more of E, D or G., which show improved performance over full length PGRN and in comparison to other GEMs individually or in combination.

In embodiments, GEM combinations including GEM B, with one or more of C, E, G, A or D, show over full length PGRN or in comparison to other GEMs individually or in combination. In embodiments, GEM combinations including GEM C, with one or more of E, G, A or D, show improved performance over full length PGRN or in comparison to other GEMs individually or in combination. In embodiments, the GEM combinations including GEM E, with one or more of G, A or D, show improved performance over full length PGRN or in comparison to other GEMs individually or in combination.

In embodiments, the GEM combinations including GEM G, with one or more of A or D, show improved performance over full length PGRN or in comparison to other GEMs individually or in combination. In embodiments, GEMs A and/or D alone or in combination show improvements.

In embodiments, GEM combinations comprising double GEM combinations of GEMs B+D, C+D, E+D, G+D, A+D, and GEM D, show enhanced Cathepsin D effects in the motor neuron cells. In embodiments, GEM combinations comprising double GEM combinations of GEMs E, C, B and/or F, show enhanced Cathepsin D effects in the motor neuron cells.

### TDP-43 Accumulation:

In preferred embodiments, the GEMs and GEM combinations of the invention exhibit improved performance in reducing TDP-43 accumulation relative to full length PGRN. In embodiments, the GEM combinations including GEM F with one or more of E, D, A or G, show improved performance over full length PGRN or in comparison to other GEMs individually or in combination. In embodiments, the GEM combinations including GEM B, with A, showed improved performance over full length PGRN and in comparison to other GEMs individually or in combination.

In embodiments, the GEM combinations including GEM F+E, F+A, or F+D, appeared to show an enhanced effect on the ability of motor neuron cells to properly clear TDP-43, even with a known TDP-43 mutation. In other embodiments, these GEM combinations cover GEMs F+E+A and/or GEMs F+E+D.

### Mini-PGRNs:

In preferred embodiments, the GEMs and GEM combinations of the invention exhibit improved performance after stable genomic incorporation in a target cell.

In embodiments, the invention relates to so-called “mini-PGRNs”, other known as GEM combinations corresponding to the amino-terminal half (GEMs GFB) or the carboxy-terminal half (GEMs CDE) of PGRN or full length human PGRN (hPGRN).

In embodiments, the GEMs and GEM combinations of the invention provide protection from serum-deprivation stress-challenges. In embodiments, GEMs E and F provide protection. In embodiments, the protective activity in CDE is at least in part effected by the E module and/or the protective activity of GFB is at least in part effected by module F.

### Functional Features:

In embodiments, the GEMs and GEM combinations of the invention provide beneficial effects in survival, using a TDP-43 cell toxicity challenge.

In embodiments, the GEMs and GEM combinations of the invention provide beneficial effects in maintenance of neuronal morphology upon serum-deprivation stress.

In embodiments, the GEMs and GEM combinations of the invention provide beneficial effects in the rate of neurite extension.

In embodiments of the invention, the beneficial functional characteristics demonstrated by the examples provided herein apply to and are considered disclosed in combination with each of the GEM combinations, and any possible combination of GEMs, falling within the scope of the disclosure. The disclosure of the present invention is intended such that each of the embodiments disclosed herein may be combined with other embodiments, for example functional or structural features disclosed for any given GEM combination may be applied for other GEM combinations.

Any disclosure of a GEM combination expressed in terms of a “+” sign between two or more GEMs may relate either to the combined presence of the GEMs in a recombinant protein or corresponding nucleic acid encoding said combination, and/or to the combined administration or preparation in spatial proximity (for example in a kit) of the GEMs or corresponding nucleic acid molecules, linked by the “+” sign.

Each of the GEM combinations disclosed in the context of a functional feature, e.g., with respect to beneficial potentially therapeutic effects, is considered disclosed both in combination with, an independently of, said functional feature. Each of the functional features disclosed herein may apply to any of the disclosed GEMs or GEM combinations, and may be supported further by one or more of the experimental approaches disclosed herein.

### Further Embodiments

Any GEM or GEM combination disclosed herein is considered disclosed in combination with its corresponding signal sequence and/or linker/leader sequence, as disclosed in the sequence listing below.

As is disclosed in the embodiments of the invention herein, the recombinant polypeptides, either administered as recombinant protein or by gene therapy, can be configured for effective delivery and therapeutic efficacy.

In embodiments, the recombinant polypeptide, or combination of multiple recombinant polypeptides, does not comprise full length PGRN.

In embodiments, the recombinant polypeptide, or combination of multiple recombinant polypeptides, does not comprise a truncation of full-length PGRN.

In embodiments, the recombinant polypeptide, or combination of multiple recombinant polypeptides, comprises a non-naturally occurring sequence, such as a recombinant or synthetic sequence, differing from the naturally occurring PGRN sequence or truncation or fragment thereof.

In embodiments, the recombinant polypeptide, or combination of multiple recombinant polypeptides, comprises a non-naturally occurring linker, leader and/or signal sequence, preferably distinct from the linker sequences in their natural order or sequence as present in full length naturally occurring PGRN.

In one embodiment, the recombinant polypeptide comprises a signal sequence positioned N-terminally of the GEMs.

In one embodiment, the recombinant polypeptide comprises one or more linker sequences positioned between the GEMs (also referred to as a leader sequence).

In these embodiments, the signal an/or linker/leader may provide improved expression, folding, cleavage and/or activity of the GEM fragments, especially when expressed in combination.

In one embodiment, the recombinant polypeptide comprises or consists of a mixed portion of a full-length PGRN sequence according to SEQ ID NO 1, and/or does not consist of the full-length PGRN sequence according to SEQ ID NO 1. These embodiments refer to the combination of GEMs, selected from the full-length PGRN protein, but not in the exact constellation of the full length PGRN protein. In embodiments, the GEM combinations described herein, as synthetic constructs, show advantages over full-length PGRN treatment.

All embodiments presented herein, relating to a recombinant polypeptide, or combination of multiple recombinant polypeptides, also relate to a therapeutic nucleic acid molecule or combination of multiple therapeutic nucleic acid molecules. The GEM combinations disclosed herein may, without limitation, be administered as proteins, or via gene therapy, i.e. by administering a nucleic acid molecule encoding the GEM(s) or combinations thereof disclosed herein.

In embodiments, the invention relates to a recombinant polypeptide comprising 2-6 GEMs. In embodiments, the invention relates to a combination of multiple recombinant polypeptides comprising 2-6 GEMs. The invention therefore relates to a single recombinant polypeptide, or a combination of multiple recombinant polypeptides, either of which is defined by the presence of the 2-6 GEMs. For example, the 2-6 GEMs may be present in separate polypeptides, but present in combination, for example for combined administration to a subject. For example, the 2-6 GEMs may be present in a single polypeptide.

These embodiments regarding 2-6 combined GEMs present in a single molecule, or present in multiple molecules but in combination, also apply to the nucleic acid molecules of the present invention.

In embodiments, the invention relates to a combination of multiple nucleic acid molecules encoding 2-6 GEMs. The invention therefore relates to a single nucleic acid molecule encoding the GEMs, or a combination of multiple nucleic acid molecules encoding the GEMs, either of which is defined by the presence of the two to six GEMs. For example, the 2-6 GEMs may be present/encoded in separate nucleic acid molecules encoding the GEMs, but present in combination, for example for combined administration to a subject. For example, the 2-6 GEMs may be present/encoded in a single nucleic acid molecule encoding the GEMs.

The nomenclature of the GEMs employed herein corresponds to the nomenclature established in the art. Alternative nomenclature may be provided herein.

### Preferred Sequences of the Invention Relate to:

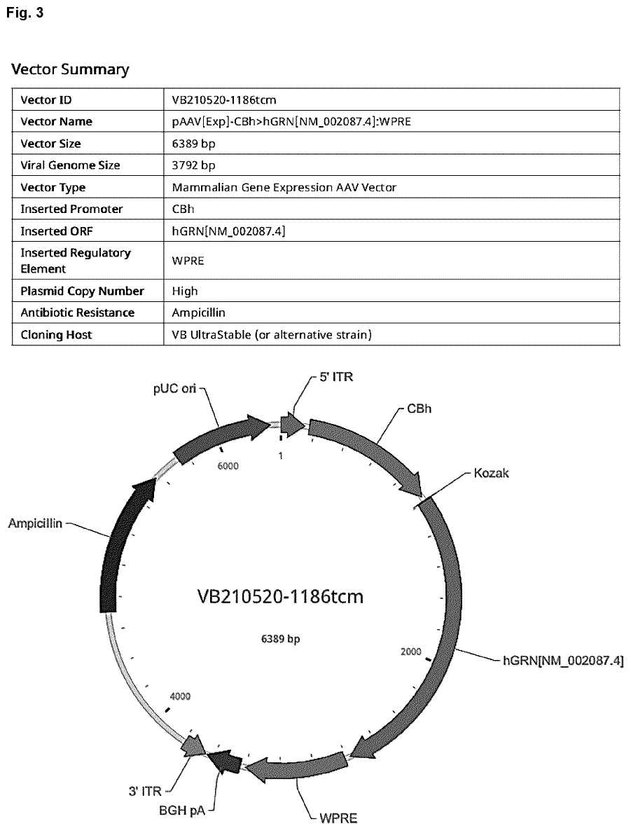

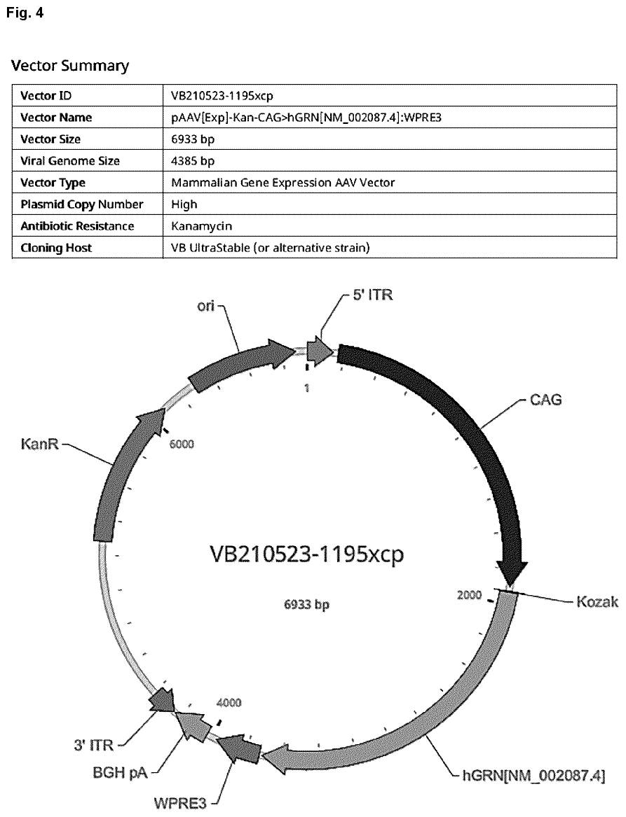

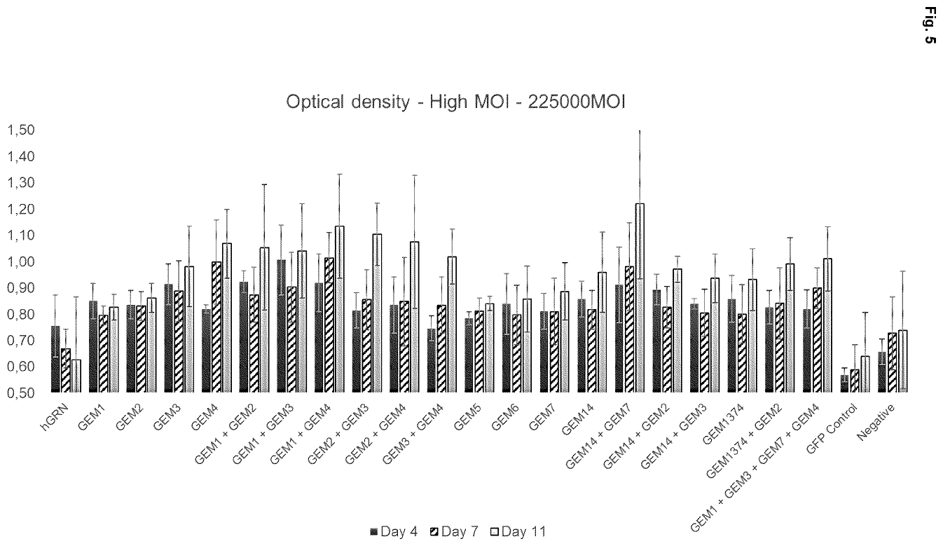

Human PGRN amino acid sequence. Length 593 aa, NP_002078.1
SEQ ID NO 1:
MWTLVSWVALTAGLVAGTRCPDGQFCPVACCLDPGGASYSCCRPLLDKWPTTLSRHLGGPCQVDA
HCSAGHSCIFTVSGTSSCCPFPEAVACGDGHHCCPRGFHCSADGRSCFQRSGNNSVGAIQCPDSQ
FECPDFSTCCVMVDGSWGCCPMPQASCCEDRVHCCPHGAFCDLVHTRCITPTGTHPLAKKLPAQR
TNRAVALSSSVMCPDARSRCPDGSTCCELPSGKYGCCPMPNATCCSDHLHCCPQDTVCDLIQSKC
LSKENATTDLLTKLPAHTVGDVKCDMEVSCPDGYTCCRLQSGAWGCCPFTQAVCCEDHIHCCPAGF
TCDTQKGTCEQGPHQVPWMEKAPAHLSLPDPQALKRDVPCDNVSSCPSSDTCCQLTSGEWGCCPI
PEAVCCSDHQHCCPQGYTCVAEGQCQRGSEIVAGLEKMPARRASLSHPRDIGCDQHTSCPVGQTC
CPSLGGSWACCQLPHAVCCEDRQHCCPAGYTCNVKARSCEKEVVSAQPATFLARSPHVGVKDVEC
GEGHFCHDNQTCCRDNRQGWACCPYRQGVCCADRRHCCPAGFRCAARGTKCLRREAPRWDAPL
RDPALRQLL
GEM F (GEM 1)
SEQ ID NO 2:
AIQCPDSQFECPDFSTCCVMVDGSWGCCPMPQASCCEDRVHCCPHGAFCDLVHTRCI
GEM B (GEM 2)
SEQ ID NO 3:
VMCPDARSRCPDGSTCCELPSGKYGCCPMPNATCCSDHLHCCPQDTVCDLIQSKCL
GEM C (GEM 3)
SEQ ID NO 4:
VPCDNVSSCPSSDTCCQLTSGEWGCCPIPEAVCCSDHQHCCPQGYTCVAEGQCQ
GEM E (GEM 4)
SEQ ID NO 5:
DVECGEGHFCHDNQTCCRDNRQGWACCPYRQGVCCADRRHCCPAGFRCAARGTKCL
GEM G (GEM 5)
SEQ ID NO 6:
GGPCQVDAHCSAGHSCIFTVSGTSSCCPFPEAVACGDGHHCCPRGFHCSADGRSCF
GEM A (GEM 6)
SEQ ID NO 7:
DVKCDMEVSCPDGYTCCRLQSGAWGCCPFTQAVCCEDHIHCCPAGFTCDTQKGTCEQ
GEM D (GEM 7)
SEQ ID NO 8:
DIGCDQHTSCPVGQTCCPSLGGSWACCQLPHAVCCEDRQHCCPAGYTCNVKARSCE
Human PGRN nucleic acid sequence, NM_002087.4 Homo sapiens granulin precursor (GRN), mRNA
SEQ ID NO 9:
GCTGCTGCCCAAGGACCGCGGAGTCGGACGCAGGCAGACCATGTGGACCCTGGTGAGCTGGG
TGGCCTTAACAGCAGGGCTGGTGGCTGGAACGCGGTGCCCAGATGGTCAGTTCTGCCCTGTGG
CCTGCTGCCTGGACCCCGGAGGAGCCAGCTACAGCTGCTGCCGTCCCCTTCTGGACAAATGGC
CCACAACACTGAGCAGGCATCTGGGTGGCCCCTGCCAGGTTGATGCCCACTGCTCTGCCGGCC
ACTCCTGCATCTTTACCGTCTCAGGGACTTCCAGTTGCTGCCCCTTCCCAGAGGCCGTGGCATG
CGGGGATGGCCATCACTGCTGCCCACGGGGCTTCCACTGCAGTGCAGACGGGCGATCCTGCTT
CCAAAGATCAGGTAACAACTCCGTGGGTGCCATCCAGTGCCCTGATAGTCAGTTCGAATGCCCG
GACTTCTCCACGTGCTGTGTTATGGTCGATGGCTCCTGGGGGTGCTGCCCCATGCCCCAGGCT
TCCTGCTGTGAAGACAGGGTGCACTGCTGTCCGCACGGTGCCTTCTGCGACCTGGTTCACACC
CGCTGCATCACACCCACGGGCACCCACCCCCTGGCAAAGAAGCTCCCTGCCCAGAGGACTAAC
AGGGCAGTGGCCTTGTCCAGCTCGGTCATGTGTCCGGACGCACGGTCCCGGTGCCCTGATGGT
TCTACCTGCTGTGAGCTGCCCAGTGGGAAGTATGGCTGCTGCCCAATGCCCAACGCCACCTGC
TGCTCCGATCACCTGCACTGCTGCCCCCAAGACACTGTGTGTGACCTGATCCAGAGTAAGTGCC
TCTCCAAGGAGAACGCTACCACGGACCTCCTCACTAAGCTGCCTGCGCACACAGTGGGGGATG
TGAAATGTGACATGGAGGTGAGCTGCCCAGATGGCTATACCTGCTGCCGTCTACAGTCGGGGG
CCTGGGGCTGCTGCCCTTTTACCCAGGCTGTGTGCTGTGAGGACCACATACACTGCTGTCCCG
CGGGGTTTACGTGTGACACGCAGAAGGGTACCTGTGAACAGGGGCCCCACCAGGTGCCCTGGA
TGGAGAAGGCCCCAGCTCACCTCAGCCTGCCAGACCCACAAGCCTTGAAGAGAGATGTCCCCT
GTGATAATGTCAGCAGCTGTCCCTCCTCCGATACCTGCTGCCAACTCACGTCTGGGGAGTGGG
GCTGCTGTCCAATCCCAGAGGCTGTCTGCTGCTCGGACCACCAGCACTGCTGCCCCCAGGGCT
ACACGTGTGTAGCTGAGGGGCAGTGTCAGCGAGGAAGCGAGATCGTGGCTGGACTGGAGAAG
ATGCCTGCCCGCCGGGCTTCCTTATCCCACCCCAGAGACATCGGCTGTGACCAGCACACCAGC
TGCCCGGTGGGGCAGACCTGCTGCCCGAGCCTGGGTGGGAGCTGGGCCTGCTGCCAGTTGCC
CCATGCTGTGTGCTGCGAGGATCGCCAGCACTGCTGCCCGGCTGGCTACACCTGCAACGTGAA
GGCTCGATCCTGCGAGAAGGAAGTGGTCTCTGCCCAGCCTGCCACCTTCCTGGCCCGTAGCCC
TCACGTGGGTGTGAAGGACGTGGAGTGTGGGGAAGGACACTTCTGCCATGATAACCAGACCTG
CTGCCGAGACAACCGACAGGGCTGGGCCTGCTGTCCCTACCGCCAGGGCGTCTGTTGTGCTGA
TCGGCGCCACTGCTGTCCTGCTGGCTTCCGCTGCGCAGCCAGGGGTACCAAGTGTTTGCGCAG
GGAGGCCCCGCGCTGGGACGCCCCTTTGAGGGACCCAGCCTTGAGACAGCTGCTGTGAGGGA
CAGTACTGAAGACTCTGCAGCCCTCGGGACCCCACTCGGAGGGTGCCCTCTGCTCAGGCCTCC
CTAGCACCTCCCCCTAACCAAATTCTCCCTGGACCCCATTCTGAGCTCCCCATCACCATGGGAG
GTGGGGCCTCAATCTAAGGCCTTCCCTGTCAGAAGGGGGTTGTGGCAAAAGCCACATTACAAGC
TGCCATCCCCTCCCCGTTTCAGTGGACCCTGTGGCCAGGTGCTTTTCCCTATCCACAGGGGTGT
TTGTGTGTGTGCGCGTGTGCGTTTCAATAAAGTTTGTACACTTTCTTAA
GEM F (GEM 1)
SEQ ID NO 10:
GCCATCCAGTGCCCCGACAGCCAGTTCGAGTGCCCCGACTTCAGCACCTGCTGCGTGATGGTG
GACGGCAGCTGGGGCTGCTGCCCCATGCCCCAGGCCAGCTGCTGCGAGGACAGGGTGCACTG
CTGCCCCCACGGCGCCTTCTGCGACCTGGTGCACACCAGGTGCATC
GEM B (GEM 2)
SEQ ID NO 11:
GTGATGTGCCCCGACGCCAGGAGCAGGTGCCCCGACGGCAGCACCTGCTGCGAGCTGCCCAG
CGGCAAGTACGGCTGCTGCCCCATGCCCAACGCCACCTGCTGCAGCGACCACCTGCACTGCTG
CCCCCAGGACACCGTGTGCGACCTGATCCAGAGCAAGTGCCTG
GEM C (GEM 3)
SEQ ID NO 12:
GTGCCCTGCGACAACGTGAGCAGCTGCCCCAGCAGCGACACCTGCTGCCAGCTGACCAGCGG
CGAGTGGGGCTGCTGCCCCATCCCCGAGGCCGTGTGCTGCAGCGACCACCAGCACTGCTGCC
CCCAGGGCTACACCTGCGTGGCCGAGGGCCAGTGCCAG
SEQ ID NO 13:
GEM E (GEM 4)
GACGTGGAGTGCGGCGAGGGCCACTTCTGCCACGACAACCAGACCTGCTGCAGGGACAACAG
GCAGGGCTGGGCCTGCTGCCCCTACAGGCAGGGCGTGTGCTGCGCCGACAGGAGGCACTGCT
GCCCCGCCGGCTTCAGGTGCGCCGCCAGGGGCACCAAGTGCCTG
GEM G (GEM 5)
SEQ ID NO 14:
GGCCCCTGCCAGGTGGACGCCCACTGCAGCGCCGGCCACAGCTGCATCTTCACCGTGAGCGG
CACCAGCAGCTGCTGCCCCTTCCCCGAGGCCGTGGCCTGCGGCGACGGCCACCACTGCTGCC
CCAGGGGCTTCCACTGCAGCGCCGACGGCAGGAGCTGCTTC
GEM A (GEM 6)
SEQ ID NO 15:
GTGAAGTGCGACATGGAGGTGAGCTGCCCCGACGGCTACACCTGCTGCAGGCTGCAGAGCGG
CGCCTGGGGCTGCTGCCCCTTCACCCAGGCCGTGTGCTGCGAGGACCACATCCACTGCTGCCC
CGCCGGCTTCACCTGCGACACCCAGAAGGGCACCTGCGAG
GEM D (GEM 7)
SEQ ID NO 16:
GACATCGGCTGCGACCAGCACACCAGCTGCCCCGTGGGCCAGACCTGCTGCCCCAGCCTGGG
CGGCAGCTGGGCCTGCTGCCAGCTGCCCCACGCCGTGTGCTGCGAGGACAGGCAGCACTGCT
GCCCCGCCGGCTACACCTGCAACGTGAAGGCCAGGAGCTGCGAG
Signal + GEM F (GEM 1)-signal: underlined, linker/leader: unmarked, GEM: bold underlined
SEQ ID NO 17:
ATGTGGACCCTGGTGAGCTGGGTGGCCCTGACCGCCGGCCTGGTGGCCGGCCAGAGGAGCGG
CAACAACAGCGTGGGCGCCATCCAGTGCCCCGACAGCCAGTTCGAGTGCCCCGACTTCAGCA
CCTGCTGCGTGATGGTGGACGGCAGCTGGGGCTGCTGCCCCATGCCCCAGGCCAGCTGCTGC
GAGGACAGGGTGCACTGCTGCCCCCACGGCGCCTTCTGCGACCTGGTGCACACCAGGTGCAT
CTAA
Signal + GEM B (GEM 2)-signal: underlined, linker/leader: unmarked, GEM: bold underlined
SEQ ID NO 18:
ATGTGGACCCTGGTGAGCTGGGTGGCCCTGACCGCCGGCCTGGTGGCCGGCACCCCCACCGG
CACCCACCCCCTGGCCAAGAAGCTGCCCGCCCAGAGGACCAACAGGGCCGTGGCCCTGAGCA
GCAGCGTGATGTGCCCCGACGCCAGGAGCAGGTGCCCCGACGGCAGCACCTGCTGCGAGCT
GCCCAGCGGCAAGTACGGCTGCTGCCCCATGCCCAACGCCACCTGCTGCAGCGACCACCTGC
ACTGCTGCCCCCAGGACACCGTGTGCGACCTGATCCAGAGCAAGTGCCTGTAA
Signal + GEM C (GEM 3)-signal: underlined, linker/leader: unmarked, GEM: bold underlined
SEQ ID NO 19:
ATGTGGACCCTGGTGAGCTGGGTGGCCCTGACCGCCGGCCTGGTGGCCGGCCAGGGCCCCCA
CCAGGTGCCCTGGATGGAGAAGGCCCCCGCCCACCTGAGCCTGCCCGACCCCCAGGCCCTGA
AGAGGGACGTGCCCTGCGACAACGTGAGCAGCTGCCCCAGCAGCGACACCTGCTGCCAGCTG
ACCAGCGGCGAGTGGGGCTGCTGCCCCATCCCCGAGGCCGTGTGCTGCAGCGACCACCAGC
ACTGCTGCCCCCAGGGCTACACCTGCGTGGCCGAGGGCCAGTGCCAGTAA
Signal + GEM E (GEM 4)-signal: underlined, linker/leader: unmarked, GEM: bold underlined
SEQ ID NO 20:
ATGTGGACCCTGGTGAGCTGGGTGGCCCTGACCGCCGGCCTGGTGGCCGGCAAGGAGGTGGT
GAGCGCCCAGCCCGCCACCTTCCTGGCCAGGAGCCCCCACGTGGGCGTGAAGGACGTGGAGT
GCGGCGAGGGCCACTTCTGCCACGACAACCAGACCTGCTGCAGGGACAACAGGCAGGGCTG
GGCCTGCTGCCCCTACAGGCAGGGCGTGTGCTGCGCCGACAGGAGGCACTGCTGCCCCGCC
GGCTTCAGGTGCGCCGCCAGGGGCACCAAGTGCCTGTAA
Signal + GEM G (GEM 5)-signal: underlined, linker/leader: unmarked, GEM: bold underlined
SEQ ID NO 21:
ATGTGGACCCTGGTGAGCTGGGTGGCCCTGACCGCCGGCCTGGTGGCCGGCAAGTGGCCCAC
CACCCTGAGCAGGCACCTGGGCGGCCCCTGCCAGGTGGACGCCCACTGCAGCGCCGGCCAC
AGCTGCATCTTCACCGTGAGCGGCACCAGCAGCTGCTGCCCCTTCCCCGAGGCCGTGGCCTG
CGGCGACGGCCACCACTGCTGCCCCAGGGGCTTCCACTGCAGCGCCGACGGCAGGAGCTGC
TTCTAA
Signal + GEM A (GEM 6)-signal: underlined, linker/leader: unmarked, GEM: bold underlined
SEQ ID NO 22:
ATGTGGACCCTGGTGAGCTGGGTGGCCCTGACCGCCGGCCTGGTGGCCGGCAGCAAGGAGAA
CGCCACCACCGACCTGCTGACCAAGCTGCCCGCCCACACCGTGGGCGACGTGAAGTGCGACA
TGGAGGTGAGCTGCCCCGACGGCTACACCTGCTGCAGGCTGCAGAGCGGCGCCTGGGGCTG
CTGCCCCTTCACCCAGGCCGTGTGCTGCGAGGACCACATCCACTGCTGCCCCGCCGGCTTCA
CCTGCGACACCCAGAAGGGCACCTGCGAGTAA
Signal + GEM D (GEM 7)-signal: underlined, linker/leader: unmarked, GEM: bold underlined
SEQ ID NO 23:
ATGTGGACCCTGGTGAGCTGGGTGGCCCTGACCGCCGGCCTGGTGGCCGGCAGGGGCAGCGA
GATCGTGGCCGGCCTGGAGAAGATGCCCGCCAGGAGGGCCAGCCTGAGCCACCCCAGGGACA
TCGGCTGCGACCAGCACACCAGCTGCCCCGTGGGCCAGACCTGCTGCCCCAGCCTGGGCGG
CAGCTGGGCCTGCTGCCAGCTGCCCCACGCCGTGTGCTGCGAGGACAGGCAGCACTGCTGCC
CCGCCGGCTACACCTGCAACGTGAAGGCCAGGAGCTGCGAGTAA
Signal + GEM F + GEM E (GEM 14)-signal: underlined, linker/leader: unmarked, GEM: bold
underlined
SEQ ID NO 24:
ATGTGGACCCTGGTGAGCTGGGTGGCCCTGACCGCCGGCCTGGTGGCCGGCCAGAGGAGCGG
CAACAACAGCGTGGGCGCCATCCAGTGCCCCGACAGCCAGTTCGAGTGCCCCGACTTCAGCA
CCTGCTGCGTGATGGTGGACGGCAGCTGGGGCTGCTGCCCCATGCCCCAGGCCAGCTGCTGC
GAGGACAGGGTGCACTGCTGCCCCCACGGCGCCTTCTGCGACCTGGTGCACACCAGGTGCAT
CAAGGAGGTGGTGAGCGCCCAGCCCGCCACCTTCCTGGCCAGGAGCCCCCACGTGGGCGTGA
AGGACGTGGAGTGCGGCGAGGGCCACTTCTGCCACGACAACCAGACCTGCTGCAGGGACAA
CAGGCAGGGCTGGGCCTGCTGCCCCTACAGGCAGGGCGTGTGCTGCGCCGACAGGAGGCAC
TGCTGCCCCGCCGGCTTCAGGTGCGCCGCCAGGGGCACCAAGTGCCTGTAA
Signal + GEM F + GEM C + GEM D + GEM E (GEM 1374)-signal: underlined, linker/leader:
unmarked, GEM: bold underlined
SEQ ID NO 25:
ATGTGGACCCTGGTGAGCTGGGTGGCCCTGACCGCCGGCCTGGTGGCCGGCCAGAGGAGCGG
CAACAACAGCGTGGGCGCCATCCAGTGCCCCGACAGCCAGTTCGAGTGCCCCGACTTCAGCA
CCTGCTGCGTGATGGTGGACGGCAGCTGGGGCTGCTGCCCCATGCCCCAGGCCAGCTGCTGC
GAGGACAGGGTGCACTGCTGCCCCCACGGCGCCTTCTGCGACCTGGTGCACACCAGGTGCAT
CCAGGGCCCCCACCAGGTGCCCTGGATGGAGAAGGCCCCCGCCCACCTGAGCCTGCCCGACC
CCCAGGCCCTGAAGAGGGACGTGCCCTGCGACAACGTGAGCAGCTGCCCCAGCAGCGACAC
CTGCTGCCAGCTGACCAGCGGCGAGTGGGGCTGCTGCCCCATCCCCGAGGCCGTGTGCTGCA
GCGACCACCAGCACTGCTGCCCCCAGGGCTACACCTGCGTGGCCGAGGGCCAGTGCCAGAG
GGGCAGCGAGATCGTGGCCGGCCTGGAGAAGATGCCCGCCAGGAGGGCCAGCCTGAGCCACC
CCAGGGACATCGGCTGCGACCAGCACACCAGCTGCCCCGTGGGCCAGACCTGCTGCCCCAG
CCTGGGCGGCAGCTGGGCCTGCTGCCAGCTGCCCCACGCCGTGTGCTGCGAGGACAGGCAG
CACTGCTGCCCCGCCGGCTACACCTGCAACGTGAAGGCCAGGAGCTGCGAGAAGGAGGTGGT
GAGCGCCCAGCCCGCCACCTTCCTGGCCAGGAGCCCCCACGTGGGCGTGAAGGACGTGGAGT
GCGGCGAGGGCCACTTCTGCCACGACAACCAGACCTGCTGCAGGGACAACAGGCAGGGCTG
GGCCTGCTGCCCCTACAGGCAGGGCGTGTGCTGCGCCGACAGGAGGCACTGCTGCCCCGCC
GGCTTCAGGTGCGCCGCCAGGGGCACCAAGTGCCTGTAA
Signal sequence:
SEQ ID NO 26:
ATGTGGACCCTGGTGAGCTGGGTGGCCCTGACCGCCGGCCTGGTGGCCGGC
Signal + linker/leader + GEM F (GEM 1)
SEQ ID NO 27:
MWTLVSWVALTAGLVAGQRSGNNSVGAIQCPDSQFECPDFSTCCVMVDGSWGCCPMPQASCCED
RVHCCPHGAFCDLVHTRCI
Signal + linker/leader + GEM B (GEM 2)
SEQ ID NO 28:
MWTLVSWVALTAGLVAGTPTGTHPLAKKLPAQRTNRAVALSSSVMCPDARSRCPDGSTCCELPSG
KYGCCPMPNATCCSDHLHCCPQDTVCDLIQSKCL
Signal + linker/leader + GEM C (GEM 3)
SEQ ID NO 29:
MWTLVSWVALTAGLVAGQGPHQVPWMEKAPAHLSLPDPQALKRDVPCDNVSSCPSSDTCCQLTS
GEWGCCPIPEAVCCSDHQHCCPQGYTCVAEGQCQ
Signal + linker/leader + GEM E (GEM 4)
SEQ ID NO 30:
MWTLVSWVALTAGLVAGKEVVSAQPATFLARSPHVGVKDVECGEGHFCHDNQTCCRDNRQGWAC
CPYRQGVCCADRRHCCPAGFRCAARGTKCL
Signal + linker/leader + GEM G (GEM 5)
SEQ ID NO 31:
MWTLVSWVALTAGLVAGKWPTTLSRHLGGPCQVDAHCSAGHSCIFTVSGTSSCCPFPEAVACGDG
HHCCPRGFHCSADGRSCF
Signal + linker/leader + GEM A (GEM 6)
SEQ ID NO 32:
MWTLVSWVALTAGLVAGSKENATTDLLTKLPAHTVGDVKCDMEVSCPDGYTCCRLQSGAWGCCPF
TQAVCCEDHIHCCPAGFTCDTQKGTCE
Signal + linker/leader + GEM D (GEM 7)
SEQ ID NO 33:
MWTLVSWVALTAGLVAGRGSEIVAGLEKMPARRASLSHPRDIGCDQHTSCPVGQTCCPSLGGSWA
CCQLPHAVCCEDRQHCCPAGYTCNVKARSCE
Signal + linker/leader + GEM F + linker/leader + GEM E (GEM 14)
SEQ ID NO 34:
MWTLVSWVALTAGLVAGQRSGNNSVGAIQCPDSQFECPDFSTCCVMVDGSWGCCPMPQASCCED
RVHCCPHGAFCDLVHTRCIKEVVSAQPATFLARSPHVGVKDVECGEGHFCHDNQTCCRDNRQGWA
CCPYRQGVCCADRRHCCPAGFRCAARGTKCL
Signal + linker/leader + GEM F + linker/leader + GEM C + linker/leader + GEM D + linker/
leader + GEM E (GEM 1374)
SEQ ID NO 35:
MWTLVSWVALTAGLVAGQRSGNNSVGAIQCPDSQFECPDFSTCCVMVDGSWGCCPMPQASCCED
RVHCCPHGAFCDLVHTRCIQGPHQVPWMEKAPAHLSLPDPQALKRDVPCDNVSSCPSSDTCCQLT
SGEWGCCPIPEAVCCSDHQHCCPQGYTCVAEGQCQRGSEIVAGLEKMPARRASLSHPRDIGCDQH
TSCPVGQTCCPSLGGSWACCQLPHAVCCEDRQHCCPAGYTCNVKARSCEKEVVSAQPATFLARSP
HVGVKDVECGEGHFCHDNQTCCRDNRQGWACCPYRQGVCCADRRHCCPAGFRCAARGTKCL
GEM D-2 (can be used in place of GEM D)
SEQ ID NO 36
AMDIGCDQHTSCPVGQTCCPSLGGSWACCQLPHAVCCEDRQHCCPAGYTCNVKARSCEKLAAALE
HHHHHH
GEM F-2 (can be used in place of GEM F)
SEQ ID NO 37
AMAIQCPDSQFECPDFSTCCVMVDGSWGCCPMPQASCCEDRVHCCPHGAFCDLVHTRCIKLAAAL
EHHHHHH
Example Vector 1 sequence for control, with full length PGRN as the expressed transgene; FIG. 3
SEQ ID NO 38
CCTGCAGGCAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAACTC
CATCACTAGGGGTTCCTTCTAGACAACTTTGTATAGAAAAGTTGCGTTACATAACTTACGGTAAAT
GGCCCGCCTGGCTGACCGCCCAACGACCCCCGCCCATTGACGTCAATAGTAACGCCAATAGGG
ACTTTCCATTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTGGCATTGT
GCCCAGTACATGACCTTATGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACCC
CCAATTTTGTATTTATTTATTTTTTAATTATTTTGTGCAGCGATGGGGGGGGGGGGGGGGGGGG
GGCGCGCGCCAGGCGGGGGGGGGCGGGGCGAGGGGGGGGGCGGGGCGAGGCGGAGAGGT
GCGGCGGCAGCCAATCAGAGCGGCGCGCTCCGAAAGTTTCCTTTTATGGCGAGGCGGCGGCG
GCGGCGGCCCTATAAAAAGCGAAGCGCGCGGCGGGCGGGAGTCGCTGCGCGCTGCCTTCGCC
CCGTGCCCCGCTCCGCCGCCGCCTCGCGCCGCCCGCCCCGGCTCTGACTGACCGCGTTACTC
CCACAGGTGAGCGGGCGGGACGGCCCTTCTCCTCCGGGCTGTAATTAGCTGAGCAAGAGGTAA
GGGTTTAAGGGATGGTTGGTTGGTGGGGTATTAATGTTTAATTACCTGGAGCACCTGCCTGAAAT
CACTTTTTTTCAGGTTGGCAAGTTTGTACAAAAAAGCAGGCTGCCACCATGTGGACCCTGGTGA
GCTGGGTGGCCTTAACAGCAGGGCTGGTGGCTGGAACGCGGTGCCCAGATGGTCAGTTCTGCC
CTGTGGCCTGCTGCCTGGACCCCGGAGGAGCCAGCTACAGCTGCTGCCGTCCCCTTCTGGACA
AATGGCCCACAACACTGAGCAGGCATCTGGGTGGCCCCTGCCAGGTTGATGCCCACTGCTCTG
CCGGCCACTCCTGCATCTTTACCGTCTCAGGGACTTCCAGTTGCTGCCCCTTCCCAGAGGCCGT
GGCATGCGGGGATGGCCATCACTGCTGCCCACGGGGCTTCCACTGCAGTGCAGACGGGCGAT
CCTGCTTCCAAAGATCAGGTAACAACTCCGTGGGTGCCATCCAGTGCCCTGATAGTCAGTTCGA
ATGCCCGGACTTCTCCACGTGCTGTGTTATGGTCGATGGCTCCTGGGGGTGCTGCCCCATGCC
CCAGGCTTCCTGCTGTGAAGACAGGGTGCACTGCTGTCCGCACGGTGCCTTCTGCGACCTGGT
TCACACCCGCTGCATCACACCCACGGGCACCCACCCCCTGGCAAAGAAGCTCCCTGCCCAGAG
GACTAACAGGGCAGTGGCCTTGTCCAGCTCGGTCATGTGTCCGGACGCACGGTCCCGGTGCCC
TGATGGTTCTACCTGCTGTGAGCTGCCCAGTGGGAAGTATGGCTGCTGCCCAATGCCCAACGC
CACCTGCTGCTCCGATCACCTGCACTGCTGCCCCCAAGACACTGTGTGTGACCTGATCCAGAGT
AAGTGCCTCTCCAAGGAGAACGCTACCACGGACCTCCTCACTAAGCTGCCTGCGCACACAGTG
GGGGATGTGAAATGTGACATGGAGGTGAGCTGCCCAGATGGCTATACCTGCTGCCGTCTACAG
TCGGGGGCCTGGGGCTGCTGCCCTTTTACCCAGGCTGTGTGCTGTGAGGACCACATACACTGC
TGTCCCGCGGGGTTTACGTGTGACACGCAGAAGGGTACCTGTGAACAGGGGCCCCACCAGGTG
CCCTGGATGGAGAAGGCCCCAGCTCACCTCAGCCTGCCAGACCCACAAGCCTTGAAGAGAGAT
GTCCCCTGTGATAATGTCAGCAGCTGTCCCTCCTCCGATACCTGCTGCCAACTCACGTCTGGGG
AGTGGGGCTGCTGTCCAATCCCAGAGGCTGTCTGCTGCTCGGACCACCAGCACTGCTGCCCCC
AGGGCTACACGTGTGTAGCTGAGGGGCAGTGTCAGCGAGGAAGCGAGATCGTGGCTGGACTG
GAGAAGATGCCTGCCCGCCGGGCTTCCTTATCCCACCCCAGAGACATCGGCTGTGACCAGCAC
ACCAGCTGCCCGGTGGGGCAGACCTGCTGCCCGAGCCTGGGTGGGAGCTGGGCCTGCTGCCA
GTTGCCCCATGCTGTGTGCTGCGAGGATCGCCAGCACTGCTGCCCGGCTGGCTACACCTGCAA
CGTGAAGGCTCGATCCTGCGAGAAGGAAGTGGTCTCTGCCCAGCCTGCCACCTTCCTGGCCCG
TAGCCCTCACGTGGGTGTGAAGGACGTGGAGTGTGGGGAAGGACACTTCTGCCATGATAACCA
GACCTGCTGCCGAGACAACCGACAGGGCTGGGCCTGCTGTCCCTACCGCCAGGGCGTCTGTTG
TGCTGATCGGCGCCACTGCTGTCCTGCTGGCTTCCGCTGCGCAGCCAGGGGTACCAAGTGTTT
GCGCAGGGAGGCCCCGCGCTGGGACGCCCCTTTGAGGGACCCAGCCTTGAGACAGCTGCTGT
GAACCCAGCTTTCTTGTACAAAGTGGGAATTCCGATAATCAACCTCTGGATTACAAAATTTGTGA
AAGATTGACTGGTATTCTTAACTATGTTGCTCCTTTTACGCTATGTGGATACGCTGCTTTAATGCC
TTTGTATCATGCTATTGCTTCCCGTATGGCTTTCATTTTCTCCTCCTTGTATAAATCCTGGTTGCT
GTCTCTTTATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAGCTGACGTCCTTTCCATGGCT
GCTCGCCTGTGTTGCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTTCCGCGTCTTCGC
CTTCGCCCTCAGACGAGTCGGATCTCCCTTTGGGCCGCCTCCCCGCATCGGGAATTCCTAGAG
CTCGCTGATCAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAGGACAGCAAGGGGG
AGGATTGGGAAGAGAATAGCAGGCATGCTGGGGAGGGCCGCAGGAACCCCTAGTGATGGAGTT
GGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCTGCCTGCAGGGGCG
CCTGATGCGGTATTTTCTCCTTACGCATCTGTGCGGTATTTCACACCGCATACGTCAAAGCAACC
ATAGTACGCGCCCTGTAGCGGCGCATTAAGCGCGGCGGGGGTGGTGGTTACGCGCAGCGTGA
CCGCTACACTTGCCAGCGCCTTAGCGCCCGCTCCTTTCGCTTTCTTCCCTTCCTTTCTCGCCAC
GTTCGCCGGCTTTCCCCGTCAAGCTCTAAATCGGGGGCTCCCTTTAGGGTTCCGATTTAGTGCT
TTACGGCACCTCGACCCCAAAAAACTTGATTTGGGTGATGGTTCACGTAGTGGGCCATCGCCCT
GATAGACGGTTTTTCGCCCTTTGACGTTGGAGTCCACGTTCTTTAATAGTGGACTCTTGTTCCAA
ACTGGAACAACACTCAACTCTATCTCGGGCTATTCTTTTGATTTATAAGGGATTTTGCCGATTTCG
GTCTATTGGTTAAAAAATGAGCTGATTTAACAAAAATTTAACGCGAATTTTAACAAAATATTAACGT
TTACAATTTTATGGTGCACTCTCAGTACAATCTGCTCTGATGCCGCATAGTTAAGCCAGCCCCGA
CACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCG
CGAGACGAAAGGGCCTCGTGATACGCCTATTTTTATAGGTTAATGTCATGATAATAATGGTTTCTT
AGACGTCAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTGAAAAAGG
AAGAGTATGAGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAACG
TTTTCCAATGATGAGCACTTTTAAAGTTCTGCTATGTGGCGCGGTATTATCCCGTATTGACGCCG
GGCAAGAGCAACTCGGTCGCCGCATACACTATTCTCAGAATGACTTGGTTGAGTACTCACCAGT
CACAGAAAAGCATCTTACGGATGGCATGACAGTAAGAGAATTATGCAGTGCTGCCATAACCATG
AGTGATAACACTGCGGCCAACTTACTTCTGACAACGATCGGAGGACCGAAGGAGCTAACCGCTT
TTTTGCACAACATGGGGGATCATGTAACTCGCCTTGATCGTTGGGAACCGGAGCTGAATGAAGC
CATACCAAACGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGATAAATCTGGA
GCCGGTGAGCGTGGAAGCCGCGGTATCATTGCAGCACTGGGGCCAGATGGTAAGCCCTCCCGT
ATCGTAGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATACTTTAGA
TTGATTTAAAACTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTAACTGGCTTCAGCA
GAGCGCAGATACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCGGGCT
GAACGGGGGGTTCGTGCACACAGCCCAGCTTGGAGCGAACGACCTACACCGAACTGAGATACC
TACAGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGCCTTTTGCTG
GCCTTTTGCTCACATGT
Example Vector 2 sequence for control, with full length PGRN as the expressed transgene FIG. 4
SEQ ID NO 39
CCTGCAGGCAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAACTC
CATCACTAGGGGTTCCTTCTAGACAACTTTGTATAGAAAAGTTGCTCGACATTGATTATTGACTAG
TTATTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTG
GCAGTACATCTACGTATTAGTCATCGCTATTACCATGGTCGAGGTGAGCCCCACGTTCTGCTTCA
CTCTCCCCATCTCCCCCCCCTCCCCACCCCCAATTTTGTATTTATTTATTTTTTAATTATTTTGTGC
AGCGATGGGGGGGGGGGGGGGGGGGGGGGGCGCGCCAGGCGGGGGGGGGGGGGGCGAGG
GGCGGGGCGGGGCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAAGCGCGCGGCGGG
CGGGAGTCGCTGCGCGCTGCCTTCGCCCCGTGCCCCGCTCCGCCGCCGCCTCGCGCCGCCCG
CCCCGGCTCTGACTGACCGCGTTACTCCCACAGGTGAGCGGGCGGGACGGCCCTTCTCCTCCG
GGCTGTAATTAGCGCTTGGTTTAATGACGGCTTGTTTCTTTTCTGTGGCTGCGTGAAAGCCTTGA
GGGGCTCCGGGAGGGCCCTTTGTGCGGGGGGAGCGGCTCGGGGGGTGCGTGCGTGTGTGTG
TGCGTGGGGAGCGCCGCGTGCGGCTCCGCGCTGCCCGGCGGCTGTGAGCGCTGCGGGCGCG
GCGCGGGGCTTTGTGCGCTCCGCAGTGTGCGCGAGGGGAGCGCGGCCGGGGGGGGTGCCCC
GCGGTGCGGGGGGGGCTGCGAGGGGAACAAAGGCTGCGTGCGGGGTGTGTGCGTGGGGGGG
TGAGCAGGGGGTGTGGGCGCGTCGGTCGGGCTGCAACCCCCCCTGCACCCCCCTCCCCGAGT
TGCTGAGCACGGCCCGGCTTCGGGTGCGGGGCTCCGTACGGGGCGTGGCGCGGGGCTCGCC
GTGCCGGGGGGGGGGGGCGGCAGGTGGGGGTGCCGGGGGGGGGGGGGCCGCCTCGGGCC
GGGGAGGGCTCGGGGGAGGGGCGCGGCGGCCCCCGGAGCGCCGGCGGCTGTCGAGGCGCG
GCGAGCCGCAGCCATTGCCTTTTATGGTAATCGTGCGAGAGGGCGCAGGGACTTCCTTTGTCC
CAAATCTGTGCGGAGCCGAAATCTGGGAGGCGCCGCCGCACCCCCTCTAGCGGGCGCGGGGC
GAAGCGGTGCGGCGCCGGCAGGAAGGAAATGGGGGGGGAGGGCCTTCGTGCGTCGCCGCGC
CGCCGTCCCCTTCTCCCTCTCCAGCCTCGGGGCTGTCCGCGGGGGGACGGCTGCCTTCGGGG
GGGACGGGGCAGGGGGGGGTTCGGCTTCTGGCGTGTGACCGGCGGCTCTAGAGCCTCTGCTA
ACCATGTTCATGCCTTCTTCTTTTTCCTACAGCTCCTGGGCAACGTGCTGGTTATTGTGCTGTCT
CATCATTTTGGCAAAGAATTGCAAGTTTGTACAAAAAAGCAGGCTGCCACCATGTGGACCCTGGT
GAGCTGGGTGGCCTTAACAGCAGGGCTGGTGGCTGGAACGCGGTGCCCAGATGGTCAGTTCTG
CCCTGTGGCCTGCTGCCTGGACCCCGGAGGAGCCAGCTACAGCTGCTGCCGTCCCCTTCTGGA
CAAATGGCCCACAACACTGAGCAGGCATCTGGGTGGCCCCTGCCAGGTTGATGCCCACTGCTC
TGCCGGCCACTCCTGCATCTTTACCGTCTCAGGGACTTCCAGTTGCTGCCCCTTCCCAGAGGCC
GTGGCATGCGGGGATGGCCATCACTGCTGCCCACGGGGCTTCCACTGCAGTGCAGACGGGCG
ATCCTGCTTCCAAAGATCAGGTAACAACTCCGTGGGTGCCATCCAGTGCCCTGATAGTCAGTTC
GAATGCCCGGACTTCTCCACGTGCTGTGTTATGGTCGATGGCTCCTGGGGGTGCTGCCCCATG
CCCCAGGCTTCCTGCTGTGAAGACAGGGTGCACTGCTGTCCGCACGGTGCCTTCTGCGACCTG
GTTCACACCCGCTGCATCACACCCACGGGCACCCACCCCCTGGCAAAGAAGCTCCCTGCCCAG
AGGACTAACAGGGCAGTGGCCTTGTCCAGCTCGGTCATGTGTCCGGACGCACGGTCCCGGTGC
CCTGATGGTTCTACCTGCTGTGAGCTGCCCAGTGGGAAGTATGGCTGCTGCCCAATGCCCAAC
GCCACCTGCTGCTCCGATCACCTGCACTGCTGCCCCCAAGACACTGTGTGTGACCTGATCCAGA
GTAAGTGCCTCTCCAAGGAGAACGCTACCACGGACCTCCTCACTAAGCTGCCTGCGCACACAGT
GGGGGATGTGAAATGTGACATGGAGGTGAGCTGCCCAGATGGCTATACCTGCTGCCGTCTACA
GTCGGGGGCCTGGGGCTGCTGCCCTTTTACCCAGGCTGTGTGCTGTGAGGACCACATACACTG
CTGTCCCGCGGGGTTTACGTGTGACACGCAGAAGGGTACCTGTGAACAGGGGCCCCACCAGGT
GCCCTGGATGGAGAAGGCCCCAGCTCACCTCAGCCTGCCAGACCCACAAGCCTTGAAGAGAGA
TGTCCCCTGTGATAATGTCAGCAGCTGTCCCTCCTCCGATACCTGCTGCCAACTCACGTCTGGG
GAGTGGGGCTGCTGTCCAATCCCAGAGGCTGTCTGCTGCTCGGACCACCAGCACTGCTGCCCC
CAGGGCTACACGTGTGTAGCTGAGGGGCAGTGTCAGCGAGGAAGCGAGATCGTGGCTGGACT
GGAGAAGATGCCTGCCCGCCGGGCTTCCTTATCCCACCCCAGAGACATCGGCTGTGACCAGCA
CACCAGCTGCCCGGTGGGGCAGACCTGCTGCCCGAGCCTGGGTGGGAGCTGGGCCTGCTGCC
AGTTGCCCCATGCTGTGTGCTGCGAGGATCGCCAGCACTGCTGCCCGGCTGGCTACACCTGCA
ACGTGAAGGCTCGATCCTGCGAGAAGGAAGTGGTCTCTGCCCAGCCTGCCACCTTCCTGGCCC
GTAGCCCTCACGTGGGTGTGAAGGACGTGGAGTGTGGGGAAGGACACTTCTGCCATGATAACC
AGACCTGCTGCCGAGACAACCGACAGGGCTGGGCCTGCTGTCCCTACCGCCAGGGCGTCTGTT
GTGCTGATCGGCGCCACTGCTGTCCTGCTGGCTTCCGCTGCGCAGCCAGGGGTACCAAGTGTT
TGCGCAGGGAGGCCCCGCGCTGGGACGCCCCTTTGAGGGACCCAGCCTTGAGACAGCTGCTG
TGAATCGAATTCTCGAGATAATCAACCTCTGGATTACAAAATTTGTGAAAGATTGACTGGTATTCT
TAACTATGTTGCTCCTTTTACGCTATGTGGATACGCTGCTTTAATGCCTTTGTATCATGCTATTGC
TTCCCGTATGGCTTTCATTTTCTCCTCCTTGTATAAATCCTGGTTAGTTCTTGCCACGGCGGAACT
CATCGCCGCCTGCCTTGCCCGCTGCTGGACAGGGGCTCGGCTGTTGGGCACTGACAATTCCGT
GGTGACCCAGCTTTCTTGTACAAAGTGGGAATTCCTAGAGCTCGCTGATCAGCCTCGACTGTGC
CTTCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGAGAATAGCAGG
CATGCTGGGGAGGGCCGCAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGCGGCC
TCAGTGAGCGAGCGAGCGCGCAGCTGCCTGCAGGGGCGCCTGATGCGGTATTTTCTCCTTACG
CATCTGTGCGGTATTTCACACCGCATACGTCAAAGCAACCATAGTACGCGCCCTGTAGCGGCGC
ATTAAGCGCGGCGGGTGTGGTGGTTACGCGCAGCGTGACCGCTACACTTGCCAGCGCCTTAGC
GCCCGCTCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAAAAACTT
GATTTGGGTGATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAACTCTATCTCG
GGCTATTCTTTTGATTTATAAGGGATTTTGCCGATTTCGGTCTATTGGTTAAAAAATGAGCTGATT
TAACAAAAATTTAACGCGAATTTTAACAAAATATTAACGTTTACAATTTTATGGTGCACTCTCAGTA
CAATCTGCTCTGATGCCGCATAGTTAAGCCAGCCCCGACACCCGCCAACACCCGCTGACGCGC
CCTGACGGGCTTGTCTGCTCCCGGCATCCGCTTACAGACAAGCTGTGACCGTCTCCGGGAGCT
GCATGTGTCAGAGGTTTTCACCGTCATCACCGAAACGCGCGAGACGAAAGGGCCTCGTGATAC
GCCTATTTTTATAGGTTAATGTCATGATAATAATGGTTTCTTAGACGTCCTGGCCCGTGTCTCAAA
ATCTCTGATGTTACATTGCACAAGATAAAAATATATCATCATGAACAATAAAACTGTCTGCTTACAT
AAACAGTAATACAAGGGGTGTTATGAGCCATATTCAACGGGAAACGTCGAGGCCGCGATTAAAT
TCCAACATGGATGCTGATTTATATGGGTATAAATGGGCTCGCGATAATGTCGGGCAATCAGGTG
CGACAATCTATCGCTTGTATGGGAAGCCCGATGCGCCAGAGTTGTTTCTGAAACATGGCAAAGG
TAGCGTTGCCAATGATGTTACAGATGAGATGGTCAGACTAAACTGGCTGACGGAATTTATGCCTC
TTCCGACCATCAAGCATTTTATCCGTACTCCTGATGATGCATGGTTACTCACCACTGCGATCCCC
GGAAAAACAGCATTCCAGGTATTAGAAGAATATCCTGATTCAGGTGAAAATATTGTTGATGCGCT
GGCAGTGTTCCTGCGCCGGTTGCATTCGATTCCTGTTTGTAATTGTCCTTTTAACAGCGATCGCG
TATTTCGTCTCGCTCAGGCGCAATCACGAATGAATAACGGTTTGGTTGATGCGAGTGATTTTGAT
GACGAGCGTAATGGCTGGCCTGTTGAACAAGTCTGGAAAGAAATGCATAAACTTTTGCCATTCTC
ACCGGATTCAGTCGTCACTCATGGTGATTTCTCACTTGATAACCTTATTTTTGACGAGGGGAAAT
TAATAGGTTGTATTGATGTTGGACGAGTCGGAATCGCAGACCGATACCAGGATCTTGCCATCCTA
TGGAACTGCCTCGGTGAGTTTTCTCCTTCATTACAGAAACGGCTTTTTCAAAAATATGGTATTGAT
AATCCTGATATGAATAAATTGCAGTTTCATTTGATGCTCGATGAGTTTTTCTATCAGAATTGGTTAA
TTGGTTGTAACACTGGCAGAGCATTACGCTGACTTGACGGGACGGCGCAAGCTCATGACCAAAA
TCCCTTAACGTGAGTTACGCGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTTTGTTTGCC
GGATCAAGAGCTACCAACTCTTTTTCCGAAGGTAACTGGCTTCAGCAGAGCGCAGATACCAAAT
ACTGTTCTTCTAGTGTAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTCGTGTCTTACCGGG
TTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGGGGAGCCTATGG
AAAAACGCCAGCAACGCGGCCTTTTTACGGTTCCTGGCCTTTTGCTGGCCTTTTGCTCACATGT

In embodiments of the invention, the recombinant polypeptide as described herein is characterized by:
a. GEM E comprises or consists of SEQ ID NO 5,
b. GEM F comprises or consists of SEQ ID NO 2,
c. GEM C comprises or consists of SEQ ID NO 4,
d. GEM D comprises or consists of SEQ ID NO 8,
e. GEM A comprises or consists of SEQ ID NO 7,
f. GEM B comprises or consists of SEQ ID NO 3.

In embodiments, the leader (signal) sequence comprises or consists of SEQ ID NO 26.

In embodiments, the linker sequence comprises or consists of one or more of the sequences presented in the table above, positioned for example between GEM coding regions, presented as unmarked sequence in the constructs SEQ ID NO 17-25, and found in SEQ ID NO 27-35, disclosed above.

The invention further relates to a recombinant polypeptide as described herein, comprising or consisting of one or more of SEQ ID NO 2, 3, 4, 5, 6, 7, 8, 36 and/or 37.

The invention further relates to a recombinant polypeptide as described herein, comprising or consisting of one or more of SEQ ID NO 27-35.

The invention further relates to nucleic acid molecules encoding a polypeptide as described herein, comprising or consisting of a sequence that encodes one or more of SEQ ID NO 2, 3, 4, 5, 6, 7, 8, 36 and/or 37.

The invention further relates to a nucleic acid molecule as described herein, comprising or consisting of a sequence according to SEQ ID NO 17, 18, 19, 20, 21, 22, 23, 24 and/or 25.

The embodiments disclosed herein regarding GEM sequences further relate to sequence variants of the sequences, as disclosed in more detail below and in the detailed description. Each sequence is considered to include sequence variants with a percentage sequence identity to the specific sequence of at least 70%, 75%, 80%, 85%, preferably 90%, more preferably at least 95%. Each sequence is also considered to include sequence variants with a truncation or extension in length, of e.g., a 0 to 10 amino acid addition or deletion at either terminus of the sequence.

In one embodiment the invention encompasses a nucleic acid molecule, and various uses thereof as described herein, selected from the group consisting of:
a) a nucleic acid molecule comprising or consisting of a nucleotide sequence that encodes one or more of a combination of multiple granulin polypeptides selected from the group consisting of GEM A, GEM B, GEM C, GEM D, GEM E, GEM F and GEM G, or any other embodiment or combination of GEMs as disclosed herein, or the nucleic acid sequences of SEQ ID NO 10-26,
b) a nucleic acid molecule comprising or consisting of a nucleotide sequence that encodes one or more of a combination of multiple granulin polypeptides selected from the group consisting of GEM A, GEM B, GEM C, GEM D, GEM E, GEM F and GEM G, wherein the length of each GEM is in between 20 and 100 amino acids preferably between 40 and 70 amino acids,
c) a nucleic acid molecule which is complementary to a nucleotide sequence in accordance with a) or b);
d) a nucleic acid molecule comprising a nucleotide sequence having sufficient sequence identity to be functionally analogous/equivalent to a nucleotide sequence according to a), b) or c), comprising preferably a sequence identity to a nucleotide sequence according to a) or b) of at least 70%, 80%, preferably 90%, more preferably 95%;
e) a nucleic acid molecule which, as a consequence of the genetic code, is degenerated into a nucleotide sequence according to a) through d); and
f) a nucleic acid molecule according to a nucleotide sequence of a) through e) which is modified by deletions, additions, substitutions, translocations, inversions and/or insertions and functionally analogous/equivalent to a nucleotide sequence according to a) through d).

One embodiment of the invention therefore relates to a polypeptide or combination thereof as described herein comprising or consisting of an amino acid sequence selected from the group consisting of:
a) the amino acid sequences disclosed herein for GEM A, GEM B, GEM C, GEM D, GEM E, GEM F and GEM G, or any other embodiment or combination of GEMs as disclosed herein, or the amino acid sequences of SEQ ID NO 2-8 or 27-35,
b) an amino acid sequence according to a) that comprises a 0 to 10 amino acid addition or deletion at the N and/or C terminus, c) an amino acid sequence comprising an amino acid sequence according to the a) or b), wherein the length of the amino acid sequence is in between 25 and 150 amino acids preferably between 40 and 100 amino acids, most preferably between 50 and 70 amino acids, amino acids,
c) an amino acid sequence having sufficient sequence identity to be functionally analogous/equivalent to an amino acid sequence according to a), or b), comprising preferably a sequence identity to an amino acid sequence according to a) of at least 70%, 80%, preferably 90%, more preferably 95%; and
d) an amino acid molecule according to an amino acid sequence of a), b), c) or d) which is modified by deletions, additions, substitutions, translocations, inversions and/or insertions and functionally analogous/equivalent to an amino acid sequence according to a), b), c) or d).

In a further aspect, the invention relates to a combination of multiple recombinant polypeptides, said combination comprising the two to six granulin/epithelin modules (GEMs).

In one embodiment, the combination of multiple recombinant polypeptides comprises GEM E (4), and additionally one or more of GEM F (1), GEM C (3) and GEM D (7).

In a further aspect, the invention relates to a nucleic acid molecule encoding a recombinant polypeptide or combination of multiple recombinant polypeptides as described herein.

In embodiments, the nucleic acid molecule is present as a combination of multiple nucleic acid molecules, each encoding one or more of the recombinant polypeptides as described herein.

In embodiments, the nucleic acid molecule as described herein is in the form of a vector configured to express the recombinant polypeptide after administration to a subject.

In embodiments, the vector is a viral vector. In embodiments, the viral vector is selected from the group consisting of an adenovirus, adeno-associated virus, lentivirus, and baculovirus.

In embodiments of the invention, the combination of features, comprising (a) a GEM combination smaller than full length PGRN, comprising 2-6 GEMs, together with (b) a viral vector, represents an improved and beneficial approach towards to PGRN administration in comparison to means of the prior art, for example administration of full length PGRN. The reduced size of a GEM combination of the invention, combined with viral administration methods, enables potentially improved delivery, transduction, expression, cleavage and/or other functional improvements, thus potentially achieving therapeutic or other functional improvements over full-length PGRN administration. Viral administration may also enable reduced toxicity compared to other modes of administration or compared to administration of full length PGRN.

In embodiments, the vector is an AAV vector, preferably an AAV9 vector, with an effective promoter, preferably with a Chicken B-actin modified promoter (CBh), and a Woodchuck hepatitis virus posttranscriptional regulatory element (WPRE).

In embodiments, the nucleic acid molecule encodes multiple GEMs configured for expression as a polycistronic mRNA, wherein said GEMs are encoded by a single nucleic acid molecule and configured for cleavage post-transcription and/or post-translation, and/or wherein the polycistronic mRNA comprises multiple internal ribosome entry sites (IRES), enabling expression of multiple distinct and soluble GEM polypeptides.

In embodiments, the nucleic acid molecule encodes multiple GEMs configured for expression under control of multiple promoters, enabling expression of multiple distinct and soluble GEM polypeptides.

A further aspect of the invention relates to a pharmaceutical composition comprising the recombinant polypeptide, the combination of multiple recombinant polypeptides, or the nucleic acid molecule as described herein, with a pharmaceutically acceptable excipient.

In one embodiment, the invention is present as a pharmaceutical combination, wherein
(a.) a first GEM is in a pharmaceutical composition in admixture with a pharmaceutically acceptable carrier, and (b.) a second and/or third GEM is in a separate pharmaceutical composition in admixture with a pharmaceutically acceptable carrier, or
(a.) a first GEM and (b.) a second and/or third GEM are present in a kit, in spatial proximity but in separate containers and/or compositions, or
(a.) a first GEM and (b.) a second and/or third GEM combined in a single pharmaceutical composition in admixture with a pharmaceutically acceptable carrier.

A further aspect of the invention relates to a method of treating a neurodegenerative disease in a subject, the method comprising administering a therapeutically effective amount of the recombinant polypeptide, the combination of multiple recombinant polypeptides, or the nucleic acid molecule as described herein to a subject in need thereof.

In embodiments, the neurodegenerative disease to be treated is selected from the group consisting of motor neuron disease, such as Amyotrophic lateral sclerosis (ALS), Frontotemporal dementia (FTD), Spinal muscular atrophy (SMA), Alzheimer's Disease (AD) and Parkinson's disease (PD).

In embodiments, the neurodegenerative disease to be treated is selected from the group consisting of dementia, schizophrenia, epilepsy, stroke, poliomyelitis, neuritis, myopathy, oxygen and nutrient deficiencies in the brain after hypoxia, anoxia, asphyxia, cardiac arrest, chronic fatigue syndrome, various types of poisoning, anaesthesia, particularly neuroleptic anaesthesia, spinal cord disorders, inflammation, particularly central inflammatory disorders, postoperative delirium and/or subsyndromal postoperative delirium, neuropathic pain, abuse of alcohol and drugs, addictive alcohol and nicotine craving, and/or effects of radiotherapy.

In embodiments, the neurodegenerative disease to be treated is selected from the group consisting of diseases associated with aberrant lysosomal function, for example Parkinson's disease (PD), Gaucher disease, or neuronal ceroid lipofuscinosis (NCL).

In embodiments, the brain disease to be treated is selected from schizophrenia and Bi-polar conditions.

In embodiments, the disease to be treated is selected from peripheral inflammatory conditions, such as arthritis and atherosclerosis.

In embodiments, the invention relates to a therapeutically effective amount of the polypeptide or combination of the invention, the combination of multiple recombinant polypeptides of the invention, the nucleic acid molecule of the invention or the composition of the invention, for use as a medicament in treating a neurodegenerative disease.

In further aspects and embodiments, the invention relates to:
A GEM polypeptide selected from the group consisting of GEM A, GEM B, GEM C, GEM D, GEM E, GEM F and GEM G.
A combination of multiple GEM polypeptides selected from the group consisting of GEM A, GEM B, GEM C, GEM D, GEM E, GEM F and GEM G.
A combination of two GEM polypeptides selected from the group consisting of GEM A, GEM B, GEM C, GEM D, GEM E, GEM F and GEM G.
A combination of three or more GEM polypeptides selected from the group consisting of GEM A, GEM B, GEM C, GEM D, GEM E, GEM F and GEM G.

In further aspects and embodiments, the invention relates to:
A nucleic acid molecule encoding a polypeptide selected from the group consisting of GEM A, GEM B, GEM C, GEM D, GEM E, GEM F and GEM G.
A nucleic acid molecule encoding a combination of multiple GEM polypeptides selected from the group consisting of GEM A, GEM B, GEM C, GEM D, GEM E, GEM F and GEM G.
A nucleic acid molecule encoding a combination of two GEM polypeptides selected from the group consisting of GEM A, GEM B, GEM C, GEM D, GEM E, GEM F and GEM G.
A nucleic acid molecule encoding a combination of three or more GEM polypeptides selected from the group consisting of GEM A, GEM B, GEM C, GEM D, GEM E, GEM F and GEM G.

In further aspects and embodiments, the invention relates to:
A pharmaceutical combination comprising a multiple, preferably 2 or 3, GEM polypeptides, or comprising a nucleic acid molecule encoding multiple, preferably 2 or 3, GEM polypeptides.
The polypeptide, nucleic acid molecule, pharmaceutical combination, or combination thereof, as described herein, preferably employ GEMs of the sequences according to SEQ ID NO 2, 3, 4, 5, 6, 7, 8, 36 and/or 37, and/or sequence variants thereof.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein or nucleic acid sequence or structure without affecting the biological or chemical action in kind or amount. These and other changes can be made to the disclosure in light of the detailed description. All such modifications are intended to be included within the scope of the appended claims.

Specific features of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### DETAILED DESCRIPTION OF THE INVENTION

The invention relates to methods and compositions comprising granulin/epithelin modules (GEMs) or combinations thereof suitable for treating neurodegenerative diseases. The invention further relates to methods of treatment of neurodegenerative diseases, such as methods of administering therapeutic recombinant GEM proteins or gene therapies for delivering recombinant GEM gene products.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application.

### Progranulin and Granulin/Epithelin Modules (GEMs);

Progranulin (PGRN) is a widely expressed, secreted glycoprotein that acts as a trophic factor for many cell types, including neuronal cells. PGRN modulates inflammation and facilitates wound repair. PGRN is involved in the regulation of multiple processes including development, wound healing, angiogenesis, growth and maintenance of neuronal cells and inflammation. In microglia, progranulin is constitutively expressed and secreted. Progranulin in neurons is important for proper trafficking and function of lysosomal enzymes such as β-glucocerebrosidase and cathepsin D.

Altered PGRN expression has been found in multiple neurodegenerative disorders, and studies into the genetic aetiology of neurodegenerative diseases have shown that heritable mutations in the PGRN gene may lead to adult-onset neurodegenerative disorders due to reduced neuronal survival. Complete PGRN deficiency and loss-of-function mutations lead to the neurodegenerative diseases or disorders, such as familial frontotemporal dementia (FTD), and the neurodegenerative lysosomal storage disorder neuronal ceroid lipofuscinosis (NCL), including neuronal ceroid lipofuscinosis 11 (CLN11). Low PGRN promotes neuroinflammation and enhances peripheral inflammatory conditions such as arthritis and atherosclerosis, and thus any disorder characterized by neuroinflammation, or peripheral inflammation could be a potential treatable disease using PGRN. In particular, low PGRN is risk factor for schizophrenia, bipolar and psychiatric disorders, in addition to AD and PD.

As used herein, the term “progranulin” or “PGRN” is used predominantly, although “progranulin” or “PGRN” may in some embodiments be used as synonyms with terms “proepithelin”, “acrogranin”, and “GP80”, which may be used herein interchangeably.

Progranulin is the precursor protein for granulins (GEMs). Cleavage of progranulin produces a variety of active about 6 kDa granulin (GEM) peptides. These smaller cleavage products are named granulin A, granulin B, granulin C, etc, or GEM A, GEM B, GEM C, etc. Epithelins 1 and 2 are synonymous with granulins A and B, respectively.

As used herein the term “granulin”, “granulin/epithelin module”, “GEM”, “epithelin”, or “Grn” may be used interchangeably, and refer to the GEMs of the present invention. References to “granulin polypeptide” also relate to a GEM polypeptide or to a combination of GEM polypeptides, as described herein. For example, the administration of “granulin polypeptide” may be used to described embodiments where a “combination of GEMs” is administered.

Cleavage of progranulin into granulins occurs either in the extracellular matrix or the lysosome. Elastase, proteinase 3 and matrix metalloproteinase are proteases capable of cleaving progranulin into individual granulin peptides. Each individual granulin domain peptide is about 60 amino acids in length. Granulin peptides are cysteine rich and capable of forming 6 disulfide bonds per residue. The disulfide bonds form a central rod-like core that shuttles each individual granulin peptide into a stacked β-sheet configuration. In humans, seven GRN (1-7) modules (GEMs) are present as tandem repeats within the precursor protein called progranulin (PGRN). Each GRN (GEM) domain consists of 12 cysteines at conserved locations. A review of granulins/GEMs is provided in Tolkatchev et al, Protein Sci. 2008 April; 17 (4): 711-724.

The GEM nomenclature used in the present disclosure and examples may include an alternative numbering scheme, as outlined in the examples.

Various combinations of two or three or more GEMs/granulins are encompassed by the present invention. By way of example, potential combinations are provided below. The position in the combination may be limiting or non-limiting, i.e., the position of the granulin may in some embodiments exist in a recombinant construct in the order presented, although this order can change.

A GEM combination may exhibit two GEMs, such combinations may be referred to as GEM dimers. A GEM combination may exhibit three GEMs, such combinations may be referred to as GEM trimers.

The combinations of two granulins are as follows:
granulin A-granulin A granulin C-granulin D granulin E-granulin G
granulin A-granulin B granulin C-granulin E granulin F-granulin A
granulin A-granulin C granulin C-granulin F granulin F-granulin B
granulin A-granulin D granulin C-granulin G granulin F-granulin C
granulin A-granulin E granulin D-granulin A granulin F-granulin D
granulin A-granulin F granulin D-granulin B granulin F-granulin E
granulin A-granulin G granulin D-granulin C granulin F-granulin F
granulin B-granulin A granulin D-granulin D granulin F-granulin G
granulin B-granulin B granulin D-granulin E granulin G-granulin A
granulin B-granulin C granulin D-granulin F granulin G-granulin B
granulin B-granulin D granulin D-granulin G granulin G-granulin C
granulin B-granulin E granulin E-granulin A granulin G-granulin D
granulin B-granulin F granulin E-granulin B granulin G-granulin E
granulin B-granulin G granulin E-granulin C granulin G-granulin F
granulin C-granulin A granulin E-granulin D granulin G-granulin G
granulin C-granulin B granulin E-granulin E
granulin C-granulin C granulin E-granulin F

The potential combinations of three granulins are as follows:
granulin A-granulin B-granulin A granulin A-granulin D-granulin A
granulin A-granulin B-granulin B granulin A-granulin D-granulin B
granulin A-granulin B-granulin C granulin A-granulin D-granulin C
granulin A-granulin B-granulin D granulin A-granulin D-granulin D
granulin A-granulin B-granulin E granulin A-granulin D-granulin E
granulin A-granulin B-granulin F granulin A-granulin D-granulin F
granulin A-granulin B-granulin G granulin A-granulin D-granulin G
granulin A-granulin C-granulin A granulin A-granulin E-granulin A
granulin A-granulin C-granulin B granulin A-granulin E-granulin B
granulin A-granulin C-granulin C granulin A-granulin E-granulin C
granulin A-granulin C-granulin D granulin A-granulin E-granulin D
granulin A-granulin C-granulin E granulin A-granulin E-granulin E
granulin A-granulin C-granulin F granulin A-granulin E-granulin F
granulin A-granulin C-granulin G granulin A-granulin E-granulin G
granulin A-granulin F-granulin A granulin B-granulin D-granulin F
granulin A-granulin F-granulin B granulin B-granulin D-granulin G
granulin A-granulin F-granulin C granulin B-granulin E-granulin A
granulin A-granulin F-granulin D granulin B-granulin E-granulin B
granulin A-granulin F-granulin E granulin B-granulin E-granulin C
granulin A-granulin F-granulin F granulin B-granulin E-granulin D
granulin A-granulin F-granulin G granulin B-granulin E-granulin E
granulin A-granulin G-granulin A granulin B-granulin E-granulin F
granulin A-granulin G-granulin B granulin B-granulin E-granulin G
granulin A-granulin G-granulin C granulin B-granulin F-granulin A
granulin A-granulin G-granulin D granulin B-granulin F-granulin B
granulin A-granulin G-granulin E granulin B-granulin F-granulin C
granulin A-granulin G-granulin F granulin B-granulin F-granulin D
granulin A-granulin G-granulin G granulin B-granulin F-granulin E
granulin B-granulin A-granulin A granulin B-granulin F-granulin F
granulin B-granulin A-granulin B granulin B-granulin F-granulin G
granulin B-granulin A-granulin C granulin B-granulin G-granulin A
granulin B-granulin A-granulin D granulin B-granulin G-granulin B
granulin B-granulin A-granulin E granulin B-granulin G-granulin C
granulin B-granulin A-granulin F granulin B-granulin G-granulin D
granulin B-granulin A-granulin G granulin B-granulin G-granulin E
granulin B-granulin C-granulin A granulin B-granulin G-granulin F
granulin B-granulin C-granulin B granulin B-granulin G-granulin G
granulin B-granulin C-granulin C granulin C-granulin A-granulin A
granulin B-granulin C-granulin D granulin C-granulin A-granulin B
granulin B-granulin C-granulin E granulin C-granulin A-granulin C
granulin B-granulin C-granulin F granulin C-granulin A-granulin D
granulin B-granulin C-granulin G granulin C-granulin A-granulin E
granulin B-granulin D-granulin A granulin C-granulin A-granulin F
granulin B-granulin D-granulin B granulin C-granulin A-granulin G
granulin B-granulin D-granulin C granulin C-granulin B-granulin A
granulin B-granulin D-granulin D granulin C-granulin B-granulin B
granulin B-granulin D-granulin E granulin C-granulin B-granulin C
granulin C-granulin B-granulin D granulin D-granulin A-granulin B
granulin C-granulin B-granulin E granulin D-granulin A-granulin C
granulin C-granulin B-granulin F granulin D-granulin A-granulin D
granulin C-granulin B-granulin G granulin D-granulin A-granulin E
granulin C-granulin D-granulin A granulin D-granulin A-granulin F
granulin C-granulin D-granulin B granulin D-granulin A-granulin G
granulin C-granulin D-granulin C granulin D-granulin B-granulin A
granulin C-granulin D-granulin D granulin D-granulin B-granulin B
granulin C-granulin D-granulin E granulin D-granulin B-granulin C
granulin C-granulin D-granulin F granulin D-granulin B-granulin D
granulin C-granulin D-granulin G granulin D-granulin B-granulin E
granulin C-granulin E-granulin A granulin D-granulin B-granulin F
granulin C-granulin E-granulin B granulin D-granulin B-granulin G
granulin C-granulin E-granulin C granulin D-granulin C-granulin A
granulin C-granulin E-granulin D granulin D-granulin C-granulin B
granulin C-granulin E-granulin E granulin D-granulin C-granulin C
granulin C-granulin E-granulin F granulin D-granulin C-granulin D
granulin C-granulin E-granulin G granulin D-granulin C-granulin E
granulin C-granulin F-granulin A granulin D-granulin C-granulin F
granulin C-granulin F-granulin B granulin D-granulin C-granulin G
granulin C-granulin F-granulin C granulin D-granulin E-granulin A
granulin C-granulin F-granulin D granulin D-granulin E-granulin B
granulin C-granulin F-granulin E granulin D-granulin E-granulin C
granulin C-granulin F-granulin F granulin D-granulin E-granulin D
granulin C-granulin F-granulin G granulin D-granulin E-granulin E
granulin C-granulin G-granulin A granulin D-granulin E-granulin F
granulin C-granulin G-granulin B granulin D-granulin E-granulin G
granulin C-granulin G-granulin C granulin D-granulin F-granulin A
granulin C-granulin G-granulin D granulin D-granulin F-granulin B
granulin C-granulin G-granulin E granulin D-granulin F-granulin C
granulin C-granulin G-granulin F granulin D-granulin F-granulin D
granulin C-granulin G-granulin G granulin D-granulin F-granulin E
granulin D-granulin A-granulin A granulin D-granulin F-granulin F
granulin D-granulin F-granulin G granulin E-granulin D-granulin E
granulin D-granulin G-granulin A granulin E-granulin D-granulin F
granulin D-granulin G-granulin B granulin E-granulin D-granulin G
granulin D-granulin G-granulin C granulin E-granulin F-granulin A
granulin D-granulin G-granulin D granulin E-granulin F-granulin B
granulin D-granulin G-granulin E granulin E-granulin F-granulin C
granulin D-granulin G-granulin F granulin E-granulin F-granulin D
granulin D-granulin G-granulin G granulin E-granulin F-granulin E
granulin E-granulin A-granulin A granulin E-granulin F-granulin F
granulin E-granulin A-granulin B granulin E-granulin F-granulin G
granulin E-granulin A-granulin C granulin E-granulin G-granulin A
granulin E-granulin A-granulin D granulin E-granulin G-granulin B
granulin E-granulin A-granulin E granulin E-granulin G-granulin C
granulin E-granulin A-granulin F granulin E-granulin G-granulin D
granulin E-granulin A-granulin G granulin E-granulin G-granulin E
granulin E-granulin B-granulin A granulin E-granulin G-granulin F
granulin E-granulin B-granulin B granulin E-granulin G-granulin G
granulin E-granulin B-granulin C granulin F-granulin A-granulin A
granulin E-granulin B-granulin D granulin F-granulin A-granulin B
granulin E-granulin B-granulin E granulin F-granulin A-granulin C
granulin E-granulin B-granulin F granulin F-granulin A-granulin D
granulin E-granulin B-granulin G granulin F-granulin A-granulin E
granulin E-granulin C-granulin A granulin F-granulin A-granulin F
granulin E-granulin C-granulin B granulin F-granulin A-granulin G
granulin E-granulin C-granulin C granulin F-granulin B-granulin A
granulin E-granulin C-granulin D granulin F-granulin B-granulin B
granulin E-granulin C-granulin E granulin F-granulin B-granulin C
granulin E-granulin C-granulin F granulin F-granulin B-granulin D
granulin E-granulin C-granulin G granulin F-granulin B-granulin E
granulin E-granulin D-granulin A granulin F-granulin B-granulin F
granulin E-granulin D-granulin B granulin F-granulin B-granulin G
granulin E-granulin D-granulin C granulin F-granulin C-granulin A
granulin E-granulin D-granulin D granulin F-granulin C-granulin B
granulin F-granulin C-granulin C granulin G-granulin B-granulin A
granulin F-granulin C-granulin D granulin G-granulin B-granulin B
granulin F-granulin C-granulin E granulin G-granulin B-granulin C
granulin F-granulin C-granulin F granulin G-granulin B-granulin D
granulin F-granulin C-granulin G granulin G-granulin B-granulin E
granulin F-granulin D-granulin A granulin G-granulin B-granulin F
granulin F-granulin D-granulin B granulin G-granulin B-granulin G
granulin F-granulin D-granulin C granulin G-granulin C-granulin A
granulin F-granulin D-granulin D granulin G-granulin C-granulin B
granulin F-granulin D-granulin E granulin G-granulin C-granulin C
granulin F-granulin D-granulin F granulin G-granulin C-granulin D
granulin F-granulin D-granulin G granulin G-granulin C-granulin E
granulin F-granulin E-granulin A granulin G-granulin C-granulin F
granulin F-granulin E-granulin B granulin G-granulin C-granulin G
granulin F-granulin E-granulin C granulin G-granulin D-granulin A
granulin F-granulin E-granulin D granulin G-granulin D-granulin B
granulin F-granulin E-granulin E granulin G-granulin D-granulin C
granulin F-granulin E-granulin F granulin G-granulin D-granulin D
granulin F-granulin E-granulin G granulin G-granulin D-granulin E
granulin F-granulin G-granulin A granulin G-granulin D-granulin F
granulin F-granulin G-granulin B granulin G-granulin D-granulin G
granulin F-granulin G-granulin C granulin G-granulin E-granulin A
granulin F-granulin G-granulin D granulin G-granulin E-granulin B
granulin F-granulin G-granulin E granulin G-granulin E-granulin C
granulin F-granulin G-granulin F granulin G-granulin E-granulin D
granulin F-granulin G-granulin G granulin G-granulin E-granulin E
granulin G-granulin A-granulin A granulin G-granulin E-granulin F
granulin G-granulin A-granulin B granulin G-granulin E-granulin G
granulin G-granulin A-granulin C granulin G-granulin F-granulin A
granulin G-granulin A-granulin D granulin G-granulin F-granulin B
granulin G-granulin A-granulin E granulin G-granulin F-granulin C
granulin G-granulin A-granulin F granulin G-granulin F-granulin D
granulin G-granulin A-granulin G granulin G-granulin F-granulin E
granulin G-granulin F-granulin F granulin G-granulin F-granulin G

### Definitions/Molecules

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, a PGRN gene or portion thereof, or regulatory elements. A nucleic acid molecule can be single-stranded or double-stranded.

A “PGRN nucleic acid” or “GEM nucleic acid” or refers to a nucleic acid that comprises the PGRN gene or a GEM portion thereof, or a functional variant of the PGRN gene or a GEM portion thereof. A functional variant of a gene includes a variant of the gene with minor variations such as, for example, silent mutations, single nucleotide polymorphisms, missense mutations, and other mutations or deletions that do not significantly alter gene function.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular PGRN protein (including fragments and portions thereof) is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the terms “gene” or “coding sequence,” is meant to refer broadly to a DNA region (the transcribed region) which encodes a protein. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into an active protein.

As used herein, “polypeptide” shall mean both peptides and proteins. In this invention, the polypeptides may be naturally occurring or recombinant (i.e., produced via recombinant DNA technology), and may contain mutations (e.g., point, insertion and deletion mutations) as well as other covalent modifications (e.g., glycosylation and labelling (via biotin, streptavidin, fluorescein, and radioisotopes)) or other molecular bonds to additional components. For example, PEGylated proteins are encompassed by the scope of the present invention. PEGylation has been widely used as a post-production modification methodology for improving biomedical efficacy and physicochemical properties of therapeutic proteins. Applicability and safety of this technology have been proven by use of various PEGylated pharmaceuticals for many years (refer Jevsevar et al, Biotechnol J. 2010 January; 5 (1): 1 13-28). In some embodiments the polypeptides described herein are modified to exhibit longer in vivo half-lives and resist degradation when compared to unmodified polypeptides. Such modifications are known to a skilled person, such as cyclized polypeptides, polypeptides fused to Vitamin B12, stapled peptides, protein lipidization and the substitution of natural L-amino acids with D-amino acids (refer Bruno et al, Ther Deliv. 2013 November; 4 (11): 1443-1467).

As used herein, a “secretion signal sequence”, or “signal peptide” (sometimes referred to as signal sequence, targeting signal, localization signal, localization sequence, transit peptide) is a short peptide (usually 16-30 amino acids long) present at the N-terminus (or occasionally C-terminus) of most newly synthesized proteins that are destined toward the secretory pathway. These proteins include those that reside either inside certain organelles (the endoplasmic reticulum, Golgi or endosomes), secreted from the cell, or inserted into most cellular membranes. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved. They are a kind of target peptide. Signal peptides function to prompt a cell to translocate the protein, usually to the cellular membrane. In prokaryotes, signal peptides direct the newly synthesized protein to the SecYEG protein-conducting channel, which is present in the plasma membrane. A homologous system exists in eukaryotes, where the signal peptide directs the newly synthesized protein to the Sec61 channel, which shares structural and sequence homology with SecYEG, but is present in the endoplasmic reticulum. Both the SecYEG and Sec61 channels are commonly referred to as the translocon, and transit through this channel is known as translocation. While secreted proteins are threaded through the channel, transmembrane domains may diffuse across a lateral gate in the translocon to partition into the surrounding membrane.

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornoe et al. (2002) Gene 297 (1-2): 21-32). In some embodiments, a promoter is operably linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34 (Database issue): D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7:2193. In some embodiments, the self-cleaving peptide is a T2A peptide.

### Sequence Variation

The embodiments disclosed herein regarding GEM sequences further relate to sequence variants of the sequences, as disclosed in more detail below and in the detailed description. Each sequence is considered to include sequence variants with a percentage sequence identity to the specific sequence of at least 70%, 75%, 80%, 85%, preferably 90%, more preferably at least 95%. Each sequence is also considered to include sequence variants with a truncation or extension in length, of e.g., a 0 to 10 amino acid addition or deletion at either terminus of the sequence.

Protein modifications to the polypeptide of the invention, which may occur through substitutions in amino acid sequence, and nucleic acid sequences encoding such molecules, are also included within the scope of the invention.

Substitutions as defined herein are modifications made to the amino acid sequence of the protein, whereby one or more amino acids are replaced with the same number of (different) amino acids, producing a protein which contains a different amino acid sequence than the primary protein. In some embodiments this amendment will not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

As is well known to those skilled in the art, altering any non-critical amino acid of a protein by conservative substitution should not significantly alter the activity of that protein because the side-chain of the amino acid which is used to replace the natural amino acid should be able to form similar bonds and contacts as the side chain of the amino acid which has been replaced. Non-conservative substitutions are possible provided that these do not excessively affect the neuroprotective or neurodegenerative activity of the polypeptide and/or reduce its effectiveness in treating neurodegenerative diseases.

As is well-known in the art, a “conservative substitution” of an amino acid or a “conservative substitution variant” of a polypeptide refers to an amino acid substitution which maintains: 1) the structure of the backbone of the polypeptide (e.g. a beta sheet or alpha-helical structure); 2) the charge or hydrophobicity of the amino acid; and 3) the bulkiness of the side chain or any one or more of these characteristics. More specifically, the well-known terminologies “hydrophilic residues” relate to serine or threonine. “Hydrophobic residues” refer to leucine, isoleucine, phenylalanine, valine or alanine. “Positively charged residues” relate to lysine, arginine or histidine. “Negatively charged residues” refer to aspartic acid or glutamic acid. Residues having “bulky side chains” refer to phenylalanine, tryptophan or tyrosine.

The terminology “conservative amino acid substitutions” is well known in the art, which relates to substitution of a particular amino acid by one having a similar characteristic (e.g., similar charge or hydrophobicity, similar bulkiness). Examples include aspartic acid for glutamic acid, or isoleucine for leucine. A conservative substitution variant will 1) have only conservative amino acid substitutions relative to the parent sequence, 2) will have at least 90% sequence identity with respect to the parent sequence, preferably at least 95% identity, 96% identity, 97% identity, 98% identity or 99% or greater identity; and 3) will retain neuroprotective or neurorestorative activity. In this regard, any conservative substitution variant of the above-described polypeptide sequences is contemplated in accordance with this invention. Such variants are considered to be “a progranulin.”

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs. Software such as BLAST or Clustal enable such sequence alignments and calculation of percent identity. As used herein, the percent homology between two sequences is equivalent to the percent identity between the sequences. Determination of percent identity or homology between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software (Altschul et al., 1990). In some embodiments, the percent homology or identity can be determined along the full-length of the nucleic acid.

A nucleic acid molecule encoding a GEM of the invention can be codon-optimized according to methods standard in the art for expression in the cell containing the target DNA of interest. For example, if the intended target nucleic acid is in a human cell, a human codon-optimized polynucleotide encoding PGRN is contemplated for use in the constructs described herein. According to some embodiments, the nucleic acid sequence is codon optimized for mammalian expression.

Modified granulin sequences, i.e. sequences that differ from the sequence encoding native granulin, are also encompassed by the invention, so long as the modified sequence still encodes a protein that exhibits the biological activity of the native granulin at a greater or lesser level of activity. These modified granulin sequences include modifications caused by point mutations, modifications due to the degeneracy of the genetic code or naturally occurring allelic variants, and further modifications that are introduced by genetic engineering, to produce recombinant granulin nucleic acids.

In embodiments, granulin nucleic acids include nucleic acids with 95% homology to the sequences described herein, or to nucleic acids which hybridize under highly stringent conditions to the complement of the DNA coding sequence for a granulin sequence as described herein. As used herein, the term “hybridization” is used in reference to the pairing of complementary nucleic acids. Hybridization and the strength of hybridization (e.g., the strength of the association between the nucleic acids) is impacted by such factors as the degree of complementary between the nucleic acids, stringency of the conditions involved, the Tm (melting temperature) of the formed hybrid, and the G:C ratio within the nucleic acids. As used herein the term “stringency” is used in reference to the conditions of temperature, ©onic strength, and the presence of other compounds such as organic solvents, under which nucleic acid hybridizations are conducted.

In an illustrative example, “highly stringent condition” can mean hybridization at 65 0C in 5×SSPE and 50% formamide and washing at 65° C. in 0.5×SSPE. In another illustrative example, “highly stringent condition” can mean hybridization at 55° C. in a hybridization buffer consisting of 50% formamide (vol/vol); 10% dextran sulfate; 1× Denhard″s solution; 20 mM sodium phosphate, pH 6.5; 5×SSC; and 200 μg of salmon sperm DNA per ml of hybridization buffer for 18 to 24 hours, and washing four times (5 min each time) with 2×SSC; 1% SDS at room temperature and then washing for 15 min at 50-55° C. with 0.1×SSC. In another illustrative example Conditions for high stringency hybridization are described in Sambrook et al., “Molecular Cloning: A Laboratory Manua””, ^{3r}d Edition, Cold Spring Harbor Laboratory Press, (2001), incorporated herein by reference. In some illustrative aspects, hybridization occurs along the full-length of the nucleic acid. Detection of highly stringent hybridization in the context of the present invention indicates strong structural similarity or structural homology (e.g., nucleotide structure, base composition, arrangement or order) to, e.g., the nucleic acids provided herein.

As used herein, the term “complementary” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement”. Complementary may be “partial” in which only some of the nucleic acid bases are matched according to the base pairing rules, or, there may be “complet” or “total” complementary between the nucleic acids.

In embodiments, the polypeptide may have, or the nucleic acid encodes a polypeptide that may have, a 0 to 10 amino acid addition or deletion at the N and/or C terminus of a sequence, with reference to the specific sequences provided herein. As used herein the term “a 0 to 10 amino acid addition or deletion at the N and/or C terminus of a sequence” means that the polypeptide may have a) 0, 1, 2, 3, 4, 5, 6, 7, 8, 9 or 10 additional amino acids at its N terminus and 0, 1, 2, 3, 4, 5, 6, 7, 8, 9 or 10 amino acids deleted at its C terminus or b) 0, 1, 2, 3, 4, 5, 6, 7, 8, 9 or 10 additional amino acids at its C terminus and 0, 1, 2, 3, 4, 5, 6, 7, 8, 9 or 10 nucleotides deleted at its N terminus, c) 0, 1, 2, 3, 4, 5, 6, 7, 8, 9 or 10 additional amino acids at its N terminus and 0, 1, 2, 3, 4, 5, 6, 7, 8, 9 or 10 additional amino acids at its N terminus or d) 0, 1, 2, 3, 4, 5, 6, 7, 8, 9 or 10 amino acids deleted at its N terminus and 0, 1, 2, 3, 4, 5, 6, 7, 8, 9 or 10 amino acids deleted at its C terminus.

Furthermore, in addition to the polypeptides described herein, peptidomimetics are also contemplated. Peptide analogues are commonly used in the pharmaceutical industry as non-peptide drugs with properties analogous to those of the template peptide. These types of non-peptide compound are termed “peptide mimetic” or “peptidomimetic” (Fauchere (1986) Adv. Drug Res. 15:29; Veber and Freidinger (1985) TINS p. 392; and Evans et al. (1987) J. Med. Chem. 30:1229) and are usually developed with the aid of computerized molecular modelling. Peptide mimetics that are structurally similar to therapeutically useful peptides may be used to produce an equivalent therapeutic or prophylactic effect. It may be preferred in some embodiments to use peptide mimetics in order to prolong the stability of the polypeptides, when administered to a subject. To this end peptide mimetics for the polypeptides may be preferred.

### Gene Therapy and Vectors for Gene Therapy:

In various illustrative embodiments, the presently described compositions comprise an (isolated and purified) nucleic acid sequence encoding the granulin or combination thereof. Methods of purifying nucleic acids are well-known to those skilled in the art. In one embodiment, the sequence is operatively linked to regulatory sequences directing expression of the granulin. In further embodiments, the sequence is operably linked to a heterologous promoter. In still further embodiments, the sequence is contained within a vector. In some embodiments, the vector is within a host cell (e.g., a neuronal cell).

As used herein, the term “vector” is used in reference to nucleic acid molecules that transfer DNA or mRNA segment(s) to cells in the patient. The vector contains the nucleic acid sequence and appropriate nucleic acid sequences necessary for the expression of the operably linked nucleic acid coding sequence in the patient. A vector is capable of expressing a nucleic acid molecule inserted into the vector and, of producing a polypeptide or protein. Nucleic acid sequences necessary for expression usually include a promoter, an operator (optional), and a ribosome binding site, often along with other sequences such as enhancers, and termination and polyadenylation signals.

In another illustrative embodiment, a granulin nucleic acid can be incorporated into a vector and administered to a patient by any protocol known in the art such as those described in U.S. Pat. Nos. 6,333,194, 7,105,342 and 7,1 12,668, incorporated herein by reference. In illustrative embodiments, granulin nucleic acid, can be introduced either in vitro into a cell extracted from an organ of the patient wherein the modified cell then being reintroduced into the body, or directly in vivo into the appropriate tissue or using a targeted vector-granulin nucleic acid construct. In various illustrative embodiments, the granulin nucleic acid can be introduced into a cell or an organ using, for example, a viral vector, a retroviral vector, or non-viral methods, such as transfection, injection of naked DNA, electroporation, sonoporation, a gene gun (e.g., by shooting DNA coated gold particles into cells using high pressure gas), synthetic oligomers, lipoplexes, polyplexes, virosomes, or dendrimers.

In one embodiment where cells or organs are treated, the granulin nucleic acid can be introduced into a cell or organ using a viral vector. The viral vector can be any viral vector known in the art. For example, the viral vector can be an adenovirus vector, a lentivirus vector, a retrovirus vector, an adeno-associated virus vector, a herpesvirus vector, a modified herpesvirus vector, and the like. In another illustrative embodiment where cells are transfected, the granulin nucleic acid can be introduced into a cell by direct DNA transfection (lipofection, calcium phosphate transfection, DEAE-dextran, electroporation, and the like).

In various illustrative embodiments, the granulin nucleic acid can be, for example, a DNA molecule, an RNA molecule, a cDNA molecule, or an expression construct comprising a granulin nucleic acid.

The granulin nucleic acids described herein can be prepared or isolated by any conventional means typically used to prepare or isolate nucleic acids and include the nucleic acids of SEQ ID. No. (1) and (13). For example, DNA and RNA molecules can be chemically synthesized using commercially available reagents and synthesizers by methods that are known in the art. The granulin nucleic acids described herein can be purified by any conventional means typically used in the art to purify nucleic acids. For example, the granulin nucleic acids can be purified using electrophoretic methods and nucleic acid purification kits known in the art (e.g. Qiagen kits). Granulin nucleic acids suitable for delivery using a viral vector or for introduction into a cell by direct DNA transfection can also be prepared using any of the recombinant methods known in the art.

The term “gene therapy” preferably refers to the transfer of DNA into a subject in order to treat a disease. The person skilled in the art knows strategies to perform gene therapy using gene therapy vectors.

Such gene therapy vectors are optimized to deliver foreign DNA into the host cells of the subject. In a preferred embodiment the gene therapy vectors may be a viral vector. Viruses have naturally developed strategies to incorporate DNA into the genome of host cells and may therefore be advantageously used. Preferred viral gene therapy vectors may include but are not limited to retroviral vectors such as Moloney murine leukemia virus (MMLV), adenoviral vectors, lentiviral, adenovirus-associated viral (AAV) vectors, pox virus vectors, herpes simplex virus vectors or human immunodeficiency virus vectors (HIV-1). However also non-viral vectors may be preferably used for the gene therapy such as plasmid DNA expression vectors driven by eukaryotic promoters or liposomes encapsulating the transfer DNA. Furthermore, preferred gene therapy vectors may also refer to methods to transfer of the DNA such as electroporation or direct injection of nucleic acids into the subject.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, an rAAV vector is single-stranded (e.g., single-stranded DNA). In some embodiments, the vector is a recombinant AAV (rAAV) vector. In some embodiments, a vector is a Baculovirus vector (e.g., an Autographa californica nuclear polyhedrosis (AcNPV) vector).

Various vectors are disclosed in the following passages. Bulcha, J. T., et al. (Viral vector platforms within the gene therapy landscape. Sig Transduct Target Ther 6, 53 (2021)) provide a useful review, the contents of which are incorporated in their entirety. The vectors disclosed therein are suitable for use in the present invention. Gene therapy is the treatment of a genetic disease by the introduction of specific cell function-altering genetic material into a patient. The key step in gene therapy is efficient gene delivery to the target tissue/cells, which is carried out by gene delivery vehicles called vectors. Contemporary viral vector-based gene therapy is achieved by in vivo delivery of the therapeutic gene into the patient by vectors based on retroviruses, adenoviruses (Ads) or adeno-associated viruses (AAVs).

### Adenovirus (Ad) Vectors:

Ad is a non-enveloped virus that is known to mostly cause infections of the upper respiratory tract but can also infect other organs such as the brain and bladder. It possesses an icosahedral protein capsid that accommodates a 26- to 45-kb linear, double-stranded DNA genome. The Ad genome is flanked by hairpin-like inverted terminal repeats (ITRs) that vary in length (30-371 bp at its termini). The ITRs serve as self-priming structures that promote primase-independent DNA replication. A packaging signal located at the left arm of the genome is required for viral genome packaging. The Ad genome encodes ˜35 proteins that are expressed in the early and late phases of viral gene transcription. The Ad genome comprises five so-called “early-phase” genes, E1A, E1B, E2, E3, and E4.7 The early-phase genes are transcribed before the initiation of viral DNA replication (about 7 h post infection). The “immediate-early” E1A gene is essential for transcription of other viral genes (e.g., E1B, E2, E3, and E4), which are responsible for viral DNA synthesis and play roles in modulating expression of host genes. E1B plays roles in counteracting the cell's activation of apoptosis by binding and inactivating p53, permitting viral replication to progress. The “late-phase” genes (L1-L5) are generally required for virus assembly, release, and lysis of the host cell. These gene products are derived from the five late transcriptional units that are produced by alternative splicing and polyadenylation of the major late messenger RNAs.

### Ad as a Vector in Gene Therapy:

Ad vectors have the following advantages: (1) high transduction efficiency, both in quiescent and dividing cells; (2) epichromosomal persistence in the host cell; (3) broad tropism for different tissue targets; and (4) and the availability of scalable production systems. Contemporary Ad vectors are derived from human serotypes hAd2 and hAd5.

First generation. The first generation of Ad vectors were engineered by replacing the E1A/E1B region with transgene cassettes that can be up to 4.5 kb in length. Removal of the E1A gene results in the inability of recombinant Ad (rAd) to replicate in the host cell.

Second generation. Due to issues with first-generation Ad vectors, researchers developed improved versions by further deleting the other early gene regions (E2a, E2b, or E4), permitting additional space for larger transgene cassettes (10.5 kb). These new vector designs include temperature-sensitive rAd vectors, generated by ablation of E2A-encoded DNA-binding protein, deletion of the E2b-encoded DNA polymerase (Pol) protein, and deletion of the E4 region.

Third generation. Third-generation Ad vectors, referred to as “gutless” or “helper-dependent” Ad vectors, have all viral sequences deleted, except for the ITRs and the packaging signal. These vectors, also called “high-capacity” adenoviral vectors (HCAds), can accommodate ˜36 kb of space for cargo gene(s). Production of HCAds in cell culture requires an additional adenoviral helper virus (HV) that is similar in composition to first-generation vectors, but with the distinction that they contain loxP sites inserted to flank the packaging signal. Compared with the previous generations of Ad vectors, HCAds have reduced immunogenicity, prolonged transduction in the host cell, and a significantly larger cargo capacity, which can accommodate multiple transgene cassettes, or therapeutic genes that are driven by their larger native promoters and enhancers to mimic physiological levels of expression.

### Aav Vectors:

Adeno-associated viruses (AAV) belong to the Parvoviridae family and more specifically constitute the Dependoparvovirus genus. As a dependoparvovirus, AAV lacks the essential genes needed for replication and expression of its own genome. These functions are provided by the Ad E1, E2a, E4, and VA RNA genes. The AAV genome itself, is a single-stranded DNA that houses four known open reading frames (ORFs). The first ORF encodes the four replication genes (rep), which are named after their molecular weights: Rep40, Rep52, Rep 68, and Rep78. The second ORF is the cap gene that encodes for the three viral capsid proteins, VP1, VP2, and VP3, respectively. The third and fourth are nested sub-genomic mRNAs, named the assembly-activating protein (AAP), which is involved in the shuttling of capsid monomers to the nucleolus where capsid assembly takes place; and the recently discovered membrane-associated accessory protein (MAAP), whose function is not completely understood. The 4.7-kb genome is flanked by 145-nt ITRs on both ends of the genome. The ITRs serve as self-priming structures for replication, and provides the signal for Rep-mediated packaging.

### AAV as a Vector for Gene Therapy:

The first demonstration of an AAV vector used in humans was performed in 1995 and involved the delivery of the cystic fibrosis transmembrane regulator (CFTR) gene packaged with the AAV2 capsid (rAAV2-CFTR), into a patient with cystic fibrosis. Since this first demonstration, multiple vector designs have been reported. The main consideration for AAV vector design is that the wild-type genome is ˜4.7 kb in size. Thus, vectors based on them are irrevocably limited to a ˜5 kb capacity. All components needed for proper expression therefore need to be abbreviated/truncated/minimized to fit into the small capsid. Alternatively, strategies that exploit ITR-mediated recombination have produced dual-vector systems that can express “oversized” transgenes, by way of transcript splicing across intermolecularly recombined ITRs from two complementary vector genomes. Other means of promoting vector-size expansion through vector recombination by homology, RNA trans-splicing, 148 or protein “trans-splicing” via split designs have also been developed.

Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

Typically, an rAAV vector (e.g., rAAV genome) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a AITR, for example as described by McCarty et al. (2003) Gene Ther. 10 (26): 2112-8.

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g. 15, 20 25, 50, 100, etc.) amino acid substitutions (e.g., mutations) relative to the wild-type AAV capsid protein from which it is derived. In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB).

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13 (16): 1935-43, Smith et al. (2009) Mol Ther 17 (11): 1888-1896, U.S. Pat. Nos. 8,945,918, 9,879,282, and International PCT Publication WO 2017/184879. However, an rAAV can be produced using any suitable method (e.g., using recombinant rep and cap genes).

Preclinical and clinical research on rAAV gene delivery in treating neurodegenerative, genetic, and acquired diseases affecting the nervous system, is well-established. Most studied in the CNS have been serotypes 1, 2, 5, 8, 9, and recombinant human (rh) 10. The effectiveness of a serotype depends on the brain region, the species, and the targeted cell type. These serotypes efficiently transduce neurons.

As disclosed in Hocquemiller et al, Human Gene Therapy, VOL 27 NUM 7 (2016), various approaches to rAAV therapy are described in the table below:

| | Clinical | | | Sero- | Pro- | | | Volume | |
|---|---|---|---|---|---|---|---|---|---|
| Disease | Trial | Status | NCT | type | moter | Gene | Dose Range | Injected | Site Injected |
| Canavan | Phase 1 | Complete | N/A | 2 | NSE | ASP | 9 × 10¹¹ | 900 | White Matter |
| Neuronal Ceroid | Phase 1 | Complete | NCT00151216 | 2 | CAG | CLN2 | 1.8-3.2 × 10¹² | 600 | White Matter |
| Lipofuscinosis | | | | | | | | | |
| Neuronal Ceroid | Phase 1/2 | Ongoing | NCT01414985 | rh10 | CAG | CLN2 | 2.85-9 × 10¹¹ | 1800 | White Matter |
| Lipofuscinosis | | | | | | | | | |
| Mucopolysaccharidos | Phase 1/2 | Complete | NCT01474343 | rh10 | PGK | SGSH | 7.2 × 10¹¹ | 720 | White Matter |
| is IIIA | | | | | | | | | |
| Mucopolysaccharidos | Phase 1/2 | Ongoing | ISRCTN19853672 | 5 | PGK | NAGLU | 4 × 10¹² | 960 | White Matter |
| is IIIB | | | | | | | | | |
| Metachromatic | Phase 1/2 | Ongoing | NCT01801709 | rh10 | CAG | ARSA | 1-4 × 10¹² | | White Matter |
| Leukodystrophy | | | | | | | | | |
| Parkinson's | Phase 2 | Complete | NCT00643890 | 2 | CAG | GAD | 2 × 10¹² | 70 | Subthalamique |
| | | | | | | | | | nucleus |
| Parkinson's | Phase 1 | Complete | NCT00229736 | 2 | CMV | AADC | 9 × 10¹⁰-3 × 10¹¹ | 200 | Striatum |
| Parkinson's | Phase 1/2 | Complete | NCT00252850 | 2 | CAG | NTN | 1.3-5.4 × 10¹¹ | 80 | Putamen |
| Parkinson's | Phase 1/2 | Ongoing | NCT00985517 | 2 | CAG | NTN | 9.4 × 10¹¹-2.4 × 10¹² | 360 | Putamen/ |
| | | | | | | | | | Substancia |
| | | | | | | | | | Nigra |
| Parkinson's | Phase 1 | Ongoing | NCT01621581 | 2 | CMV | GONF | 9 × 10¹⁰-3 × 10¹² | | Striatum |
| Parkinson's | Phase 1 | Ongoing | NCT01973543 | 2 | | AADC | 7.5 × 10¹¹-1.5 × 10¹² | | Striatum |
| Parkinson's | Phase 1/2 | Ongoing | NCT02418598 | 2 | | AADC | 3-9 × 10¹¹ | 200-600 | Putamen |
| Parkinson's | Phase 1 | Ongoing | NCT01395641 | 2 | | AADC | | | Putamen |
| Alzheimer's | Phase 1 | Complete | NCT00087789 | 2 | CAG | NGF | 1.2 × 10¹⁰-1.2 × 10¹¹ | 40-80 | Nucleus Basalis |
| Alzheimer's | Phase 2 | N/A | NCT00876863 | 2 | CAG | NGF | 2 × 10¹¹ | | Intraparenchymal |
| Giant Axonal | Phase 1 | Ongoing | NCT02362438 | 9 | JeT | Gigaxonin | | | Intrathecal |
| Neuropathy | | | | | | | | | |
| CLN6 Disease | Phase 1/2 | Ongoing | NCT02725580 | 9 | CAG | CLN2 | 1.5 × 10¹³ | | Lombar |
| SMAI | Phase 1/2 | Ongoing | NCT02122952 | 9 | CAG | SMN | 6.7 × 10¹³-3.3 × 10¹⁴ | | Peripheral Vein |
| | | | | | U1a | | | | |
| Mucopolysaccharidos | Phase 1/2 | Ongoing | NCT02716246 | 9 | | SGSH | 5 × 10¹²-1 × 10¹³ | | Peripheral Vein |
| is IIIA | | | | | | | | | |

Any of the Serotypes, promoters, dose ranges, volumes, or sites of injection may be employed in the present invention. For example, in embodiments of the invention, the NSE, CAG, PGK, CMV, Jet or CAG U1a promoters may be employed. Injection sites of the rAAV, in embodiments, may be selected from white matter, subthalamic nucleus, striatum, Putamen, Nigra, Nucleus Basalis, intrathecal, lumbar, or peripheral vein.

### Lentivirus Vectors:

Lentiviruses constitute a genus of the retroviridae family. Retroviruses are spherical, enveloped, single-stranded RNA viruses that are ˜100 nm in diameter. The lentiviral particle encapsidates two sense-strand RNAs that are bound by nucleocapsid proteins. The particle also contains reverse transcriptase, integrase, and protease proteins. Retroviruses can be classified into simple or complex viruses, based on their genome organization. Gammaretroviruses are an example of simple retroviruses, whereas the HIV-1, a lentivirus, is an example of a complex retrovirus.

### Lentiviruses as Vectors in Gene Therapy:

Lentiviral vectors have several features that make them amenable to transgene delivery for therapeutic purposes. Lentiviral vectors are integrating vectors that permit long-term transgene expression. They have a packaging capacity of up to 9 kb. High-level expression of multiple genes may be a requisite for achieving therapeutic outcomes for certain diseases. Employing two separate vectors carrying co-dependent transgenes may not be an optimal solution, as successful transduction of multiple viral vectors to the same cell is not efficient. Lentiviral vectors are demonstrated to have the ability to express multiple genes from a single vector. Lentiviral vectors can transduce postmitotic and quiescent cells, whereas other retrovirus-based platforms, such as gammaretroviral vectors, require active cell division for successful infection.

Lentiviral vector systems that are derived from the HIV-1 virus have evolved through the years. These advancements have been made in part to mitigate the potential risks associated with the virus that the platform is based on. The first generation of HIV-1-based vectors retained most of the viral genome within the trans packaging construct, including the viral core, regulatory protein coding sequences, and accessory regulatory genes. Additional modifications have been made to improve the expression and transduction efficiency of lentiviral vectors. Incorporating transcriptional regulatory elements, such as a central polypurine tract (cppt) and a matrix attachment region (MAR) in the cis expression vector augments viral transduction. In addition, incorporating woodchuck hepatitis virus post-transcriptional regulatory element (WPRE) as a posttranscriptional regulatory element in the 3′-untranslated region of the ORF significantly enhances transgene expression.

### Compositions, Dose, and Routes of Administration:

The polypeptides, nucleic acid molecules, gene therapy vectors or cells described herein may comprise different types of carriers depending on whether they are to be administered in solid, liquid or aerosol form, and whether they need to be sterile for such routes of administration as injection.

The active agent present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, directly, via a catheter, via a lavage, in cremes, in lipid compositions (e.g., liposomes), locally applied by sponges or by other method or any combination of the forgoing as would be known to one of ordinary skill in the art (see, for example, Remingto″s Pharmaceutical Sciences, 18th Ed. Mack Printing Company, 1990, incorporated herein by reference).

The present invention encompasses treatment of a patient by introducing a therapeutically effective number polypeptides, nucleic acids, gene therapy vectors or cells into a subject's bloodstream. As used herein, “introducing” polypeptides, nucleic acids, gene therapy vectors or cells into the subject's bloodstream shall include, without limitation, introducing such polypeptides, nucleic acids, gene therapy vectors or cells into one of the subject's veins or arteries via injection. Such administering can also be performed, for example, once, a plurality of times, and/or over one or more extended periods. A single injection is preferred, but repeated injections over time (e.g., quarterly, half-yearly or yearly) may be necessary in some instances. Such administering is also preferably performed using an admixture of polypeptides, nucleic acids, gene therapy vectors or cells and a pharmaceutically acceptable carrier. Pharmaceutically acceptable carriers are well known to those skilled in the art and include, but are not limited to, 0.01-0.1 M and preferably 0.05 M phosphate buffer or 0.8% saline.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Additionally, such pharmaceutically acceptable carriers can be aqueous or non-aqueous solutions, suspensions, and emulsions, most preferably aqueous solutions. Aqueous carriers include water, alcoholic/aqueous solutions, emulsions and suspensions, including saline and buffered media. Parenteral vehicles include sodium chloride solution, Ringers dextrose, dextrose and sodium chloride, lactated Ringers and fixed oils. Intravenous vehicles include fluid and nutrient replenishers, electrolyte replenishers such as Ringers dextrose, those based on Ringers dextrose, and the like. Fluids used commonly for i.v. administration are found, for example, in Remington: The Science and Practice of Pharmacy, 20th Ed., p. 808, Lippincott Williams S-Wilkins (2000). Preservatives and other additives may also be present, such as, for example, antimicrobials, antioxidants, chelating agents, inert gases, and the like.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Examples of parenteral dosage forms include aqueous solutions of the active agent, in an isotonic saline, 5% glucose or other well-known pharmaceutically acceptable liquid carriers such as liquid alcohols, glycols, esters, and amides. The parenteral dosage form in accordance with this invention can be in the form of a reconstitutable lyophilizate comprising a dose of a composition comprising granulin. In one aspect of the present embodiment, any of a number of prolonged or sustained release dosage forms known in the art can be administered such as, for example, the biodegradable carbohydrate matrices described in U.S. Pat. Nos. 4,713,249; 5,266,333; and 5,417,982, the disclosures of which are incorporated herein by reference.

In an illustrative embodiment pharmaceutical formulations for general use with granulins for parenteral administration comprising: a) a pharmaceutically active amount of the granulin; b) a pharmaceutically acceptable pH buffering agent to provide a pH in the range of about pH 4.5 to about pH 9; c) an ionic strength modifying agent in the concentration range of about 0 to about 250 millimolar; and d) water soluble viscosity modifying agent in the concentration range of about 0.5% to about 7% total formula weight are described or any combinations of a), b), c) and d).

In various illustrative embodiments, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, Cacodylate, MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. It is preferred that non-acidic viscosity enhancing agents, such as a neutral or basic agent be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed above. In one preferred embodiment, the viscosity modulating agent is cellulose that has been modified such as by etherification or esterification.

In various illustrative embodiments, granulin compositions are provided that may comprise all or portions of granulin polypeptides, alone or in combination with at least one other agent, such as an excipient and/or a stabilizing compound and/or a solubilizing agent, and may be administered in any sterile, biocompatible pharmaceutical carrier, including, but not limited to, saline, buffered saline, dextrose, glucose, and water. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including tragacanth; and proteins such as gelatin and collagen. Suitable disintegrating or solubilizing agents include agar, alginic acid or a salt thereof such as sodium alginate.

In illustrative embodiments, granulin polypeptides can be administered to a patient alone, or in combination with other agents, drugs or hormones or in pharmaceutical compositions where it is mixed with excipient(s) or other pharmaceutically acceptable carriers. In one embodiment, the pharmaceutically acceptable carrier is pharmaceutically inert. In another embodiment, granulin polypeptides may be administered alone to a patient suffering from a neurological disease.

The unitary daily dosage of the composition comprising the granulin polypeptide can vary significantly depending on the patient condition, the disease state being treated, the route of administration of granulin and tissue distribution, and the possibility of co-usage of other therapeutic treatments. The effective amount of a granulin to be administered to the patient is based on body surface area, patient weight, physician assessment of patient condition, and the like.

In one illustrative embodiment, an effective dose of a granulin can range from about 1 ng/kg of patient body weight to about 1 mg/kg of patient body weight, more preferably from about 1 ng/kg of patient body weight to about 500 ng/kg of patient body weight, and most preferably from about 1 ng/kg of patient body weight to about 100 ng/kg of patient body weight.

In another illustrative embodiment, an effective dose of the granulin polypeptide can range from about 1 μg/kg of patient body weight to about 1 mg/kg of patient body weight. In various illustrative embodiments, an effective dose can range from about 1 μg/kg of patient body weight to about 500 ng/kg of patient body weight, from about 500 μg/kg of patient body weight to about 500 ng/kg of patient body weight, from about 1 ng/kg of patient body weight to about 500 ng/kg of patient body weight, from about 100 ng/kg of patient body weight to about 500 ng/kg of patient body weight, and from about 1 ng/kg of patient body weight to about 100 ng/kg of patient body weight.

In another illustrative embodiment, an effective dose of the granulin polypeptide can range from about 1 μg/kg of patient body weight to about 1 mg/kg of patient body weight. In various illustrative embodiments, an effective dose can range from about 1 μg/kg of patient body weight to about 500 μg/kg of patient body weight, from about 500 ng/kg of patient body weight to about 500 μg/kg of patient body weight, from about 1 μg/kg of patient body weight to about 500 μg/kg of patient body weight, from about 0.1 μg/kg of patient body weight to about 5 μg/kg of patient body weight, from about 0.1 μg/kg of patient body weight to about 10 μg/kg of patient body weight, and from about 0.1 μg/kg of patient body weight to about 100 μg/kg of patient body weight.

In another illustrative embodiment, an effective dose of the nucleic acid molecule can range from about 1 million nucleic acid molecule molecules per 70 kg patient body to about 1 billion nucleic acid molecule molecules per 70 kg patient body. In various illustrative embodiments, an effective dose can range from about 1 million nucleic acid molecule molecules per 70 kg patient body to about 500 million nucleic acid molecule molecules per 70 kg patient body, from about 200,000 nucleic acid molecule molecules per 70 kg patient body to about 200 million nucleic acid molecule molecules per 70 kg patient body, from about 1 million nucleic acid molecule molecules per 70 kg patient body to about 200 million nucleic acid molecule molecules per 70 kg patient body.

The composition comprising the granulin polypeptide can be adapted for parenteral administration, the route of parenteral administration can be selected from the group consisting of intradermally, subcutaneously, intramuscularly, intraperitoneally, intravenously, intraventricularly, intrathecally, intracerebrally, and intracordally.

In some embodiments, a composition is administered directly to the nervous system, the CNS or peripheral nervous system of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

As used herein, the term “nervous system” includes both the central nervous system and the peripheral nervous system. The term “central nervous system” or “CNS” includes all cells and tissue of the brain and spinal cord of a vertebrate. The term “peripheral nervous system” refers to all cells and tissue of the portion of the nervous system outside the brain and spinal cord. Thus, the term “nervous system” includes, but is not limited to, neuronal cells, glial cells, astrocytes, cells in the cerebrospinal fluid (CSF), cells in the interstitial spaces, cells in the protective coverings of the spinal cord, epidural cells (i.e., cells outside of the dura mater), cells in non-neural tissues adjacent to or in contact with or innervated by neural tissue, cells in the epineurium, perineurium, endoneurium, funiculi, fasciculi, and the like. In some embodiments, the polypeptides or nucleic acids are to be delivered to target cells, wherein target cells comprise preferably neuronal cells.

In a preferred embodiment the pharmaceutical composition for use as a medicament as described herein is administered by introducing a therapeutically effective amount of the composition into the blood stream of a subject. This route of administration is particularly advantageous for an administration of the polypeptides. Advantageously the polypeptides and in particular the soluble polypeptides as described herein can cross the blood-brain barrier. Therefore a systemic administration by introducing a therapeutically effective amount of the polypeptides into the vascular system may be used to treat neurodegeneration in the brain.

In a further preferred embodiment the pharmaceutical composition for use as a medicament as described herein is administered locally. It may also be preferred that the local administration of the pharmaceutical composition to the skin is achieved by cremes or lotions that comprise the gene therapy or polypeptides.

Moreover in a preferred embodiment the local administration of the polypeptides may be preferably mediated by an implant such as a collagen sponge. In further preferred embodiment the polypeptides may be locally administered by means of a hydrogel. Hydrogels are three-dimensional, cross-linked networks of water-soluble polymers. The person skilled in the art knows how to produce suitable hydrogels for the delivery of proteins or polypeptides (Hoare et al. 2008, Peppas et al. 2000, Hoffmann A. et al. 2012). In particular the density of the cross-linked network of the hydrogel may be advantageously optimized to achieve a porosity suited to load the polypeptides into the hydrogel. Subsequently the release of the polypeptides is governed by the diffusion of the peptides throughout the gel network. Therefore the release rate and thus the therapeutically effective amount of the polypeptides can be precisely tuned by optimizing the cross-linking density of the hydrogel. Moreover preferred hydrogels may also encompass an outer membrane optimized for the release of the polypeptides. The preferred hydrogels are biocompatible and are preferably implanted for a long term local supply of the polypeptides.

Any effective regimen for administering the composition comprising granulin can be used. For example, the composition comprising granulin can be administered as a single dose, or the composition comprising granulin can be divided and administered as a multiple-dose daily regimen. Further, a staggered regimen, for example, one to three days per week can be used as an alternative to daily treatment, and for the purposes of this invention such intermittent or staggered daily regimen is considered to be equivalent to every day treatment and within the scope of this invention. In one embodiment, the patient is treated with multiple injections of the composition comprising granulin to decrease neuronal cell death. In another embodiment, the patient is injected multiple times (e.g., about 2 up to about 50 times) with the composition comprising granulin, for example, at 12-72 hour intervals or at 48-72 hour intervals. Additional injections of the composition comprising granulin can be administered to the patient at an interval of days or months after the initial injections(s) and the additional injections prevent recurrence of disease. Alternatively, the initial injection(s) of the composition comprising granulin may prevent recurrence of disease.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered either peripherally or directly to the CNS of a subject, or both peripherally and directly to the CNS of a subject. For example, in some embodiments, a subject is administered a composition by intra-arterial injection (e.g., injection into the carotid artery) and/or by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 109 Genome copies (GC)/kg and about 1014 GC/kg (e.g., about 109 GC/kg, about 1010 GC/kg, about 10¹¹ GC/kg, about 10¹² GC/kg, about 10¹² GC/kg, or about 1014 GC/kg). In some embodiments, a subject is administered a high titer (e.g., >10¹² Genome Copies GC/kg of an rAAV) by injection to the CSF space, or by intraparenchymal injection.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### Medical Conditions:

In embodiments, the neurodegenerative disease to be treated is selected from the group consisting of motor neuron disease, Amyotrophic lateral sclerosis (ALS), Frontotemporal dementia (FTD), Spinal muscular atrophy (SMA), Alzheimer's Disease (AD) and Parkinson's disease (PD). In other embodiments, the neurodegenerative disease to be treated is selected from the group consisting of dementia, schizophrenia, epilepsy, stroke, poliomyelitis, neuritis, myopathy, oxygen and nutrient deficiencies in the brain after hypoxia, anoxia, asphyxia, cardiac arrest, chronic fatigue syndrome, various types of poisoning, anaesthesia, particularly neuroleptic anaesthesia, spinal cord disorders, inflammation, particularly central inflammatory disorders, postoperative delirium and/or subsyndromal postoperative delirium, neuropathic pain, abuse of alcohol and drugs, addictive alcohol and nicotine craving, and/or effects of radiotherapy. In embodiments, the neurodegenerative disease to be treated is selected from the group consisting of diseases associated with aberrant lysosomal function, for example Parkinson's disease (PD), Gaucher disease, or neuronal ceroid lipofuscinosis (NCL). In embodiments, the brain disease to be treated is selected from schizophrenia and Bi-polar conditions. In embodiments, the disease to be treated is selected from peripheral inflammatory conditions, such as arthritis and atherosclerosis.

In another illustrative embodiment, a pharmaceutical composition comprising therapeutically effective amounts of granulin polypeptide or combinations thereof and a pharmaceutically acceptable carrier is provided, wherein the therapeutically effective amounts comprise amounts capable to increase neuronal cell survival in a patient and/or reducing neuronal cell death in the patient and/or reducing or preventing the symptoms of the neurodegenerative disease in the patient.

In another illustrative embodiment, a method for reducing neuronal cell death in a patient is provided, the method comprising the steps of administering to a patient a therapeutically effective amount of a granulin polypeptide or combinations thereof, wherein the amount of the peptide is effective to increase neuronal cell survival in a patient and/or reducing neuronal cell death in the patient and/or reducing or preventing the symptoms of the neurodegenerative disease in the patient.

In another illustrative embodiment, a method for treating a patient with a neurodegenerative disease is provided, the method comprising the steps of administering to the patient a composition comprising a granulin polypeptide or combinations thereof, and reducing neuronal cell death in the patient and/or reducing or preventing the symptoms of the neurodegenerative disease in the patient.

In another illustrative embodiment, a pharmaceutical composition comprising therapeutically effective amounts of nucleic acid molecule that expresses one or more granulins and a pharmaceutically acceptable carrier is provided, wherein the therapeutically effective amounts comprise amounts capable to increase neuronal cell survival in a patient and/or reducing neuronal cell death in the patient and/or reducing or preventing the symptoms of the neurodegenerative disease in the patient.

In another illustrative embodiment, a method for reducing neuronal cell death in a patient is provided, the method comprising the steps of administering to a patient a therapeutically effective amount of an nucleic acid molecule that expresses one or more granulins, wherein the amount of the nucleic acid molecule is effective to increase neuronal cell survival in a patient and/or reducing neuronal cell death in the patient and/or reducing or preventing the symptoms of the neurodegenerative disease in the patient.

In another illustrative embodiment, a method for treating a patient with a neurodegenerative disease is provided, the method comprising the steps of administering to the patient a composition comprising a nucleic acid molecule that expressions one or more granulins, and increasing neuronal cell survival in a patient and/or reducing neuronal cell death in the patient and/or reducing or preventing the symptoms of the neurodegenerative disease in the patient.

In one illustrative aspect, the neurodegenerative disease state can include, but is not limited to, Parkinson's disease and the parkinsonisms including progressive supranuclear palsy, Alzheimer's disease, and motor neuron disease (e.g., amyotrophic lateral sclerosis); or any other neurodegenerative disease mediated by an increase in neuronal cell death and/or a modification of progranulin expression and/or function.

In illustrative embodiments, the neurodegenerative disease is mediated by a heritable mutation of the progranulin gene that modifies progranulin expression. For example, Frontotemporal dementia (FTD), or frontotemporal degeneration disease, or frontotemporal neurocognitive disorder encompasses several types of dementia involving the frontal and temporal lobes. FTDs are broadly presented as behavioral or language disorders. The three main subtypes or variant syndromes are a behavioral variant (bvFTD) previously known as Pick's disease, and two variants of primary progressive aphasia-semantic variant (svPPA), and nonfluent variant (nfvPPA). Two rare distinct subtypes of FTD are neuronal intermediate filament inclusion disease (NIFID), and basophilic inclusion body disease. Other related disorders include corticobasal syndrome and FTD with amyotrophic lateral sclerosis (ALS) FTD-ALS also called FTD-MND.

Progranulin was found as a major genetic cause of frontotemporal dementia (FTD) in 2006, only months before TDP-43 was identified as the main protein constituent of the histopathological lesions in the same patients. Up to that point, the only known FTD gene had been MAPT, located near the progranulin gene, GRN, on chromosome 17q21, and the twin discoveries broke a double logjam in the field. Roughly 70 GRN mutations are known explain all 17q21-linked autosomal-dominant FTD families not accounted for by tau mutations, and because all FTD patients with a GRN mutation have TDP-43 pathology, TDP-43 explains these family's tau-negative protein inclusions. GRN mutations explain up to 20 percent of familial and 5 percent of sporadic FTD. Histopathological commonalities notwithstanding, GRN mutations lead to a variety of clinical presentations, causing mostly behavioural FTD and progressive nonfluent aphasia, but also rare presentations of Alzheimer's disease or parkinsonism.

All pathologic GRN mutations reduce progranulin levels or result in loss of function. Indeed, blood progranulin levels indicate the presence of a pathogenic progranulin mutation and are rapidly becoming a diagnostic biomarker. Progranulin is a secreted growth factor known for its role in biological processes such as inflammation, wound healing, and cancer, and for its neurotrophic properties. It is proteolytically processed into peptides called granulins. The present invention is therefore based, in some embodiments, on improved granulin treatment for FTD.

Several factors are known to influence progranulin expression. They include intrinsic factors, for example the gene TMEM106B and various microRNAs, as well as pharmacological agents, such as the histone deacetylase inhibitor SAHA and certain alkalizing drugs. Agents targeting the endocytic progranulin receptor sortilin-1 appear to increase plasma progranulin levels by slowing its internalization. Homozygous GRN mutations cause the rare lysosomal storage disease ceroid lipofuscinosis, and progranulin localizes to intraneuronal membrane compartments, including lysosomes. Both homozygous and heterozygous GRN knockout mice exist; the former show both behavioral and inflammatory phenotypes, the latter develop only the former.

Frontotemporal dementias are mostly early-onset syndromes that are linked to frontotemporal lobar degeneration (FTLD), which is characterized by progressive neuronal loss predominantly involving the frontal or temporal lobes, and a typical loss of over 70% of spindle neurons, while other neuron types remain intact. FTD was first described by Arnold Pick in 1892 and was originally called Pick's disease, a term now reserved only for behavioural variant FTD which shows the presence of Pick bodies and Pick cells. Second only to Alzheimer's disease (AD) in prevalence, FTD accounts for 20% of early-onset dementia cases. Signs and symptoms typically manifest in late adulthood, more commonly between the ages of 45 and 65, approximately equally affecting men and women. Common signs and symptoms include significant changes in social and personal behaviour, apathy, blunting of emotions, and deficits in both expressive and receptive language. Currently, there is no cure for FTD, but there are treatments that help alleviate symptoms.

In illustrative embodiments, the neurodegenerative disease is mediated by an environmental insult to the patient. As used herein, a neurodegenerative disease mediated by an environmental insult to the patient means a disease that is caused by an environmental insult and is not caused by a heritable mutation of the progranulin gene that modifies progranulin expression. A heritable mutation is a permanent mutation in a patient's DNA that may be transmitted to the patient's offspring.

These illustrative embodiments are however not meant to exclude the influence of allelic variants of modifier genes, that are, for example, involved in the metabolism of the neurotoxin, that render an individual more or less sensitive to neurodegenerative disease development. As used herein these modifier genes can modify the course of disease development.

The neurodegenerative disease mediated by environmental insult to the patient may be a sporadic disease linked to environmental factors that cause neuronal cell death directly or indirectly by modifying gene expression. In various other illustrative embodiments, the environmental insult is derived from the patient's diet or is the result of endogenous synthesis, or both. In one illustrative embodiment, the environmental insult causes synthesis of a compound that causes a detrimental effect in vivo. The neuronal cell death may occur by any variety of means including, but not limited to, excitotoxicity or oxidative stress.

In another illustrative embodiment, the neurodegenerative disease state is mediated by an excitotoxin. Excitotoxins are a class of substances that damage neurons through overactivation of receptors, for example, receptors for the excitatory neurotransmitter glutamate, leading to neuronal cell death. Examples of excitotoxins include excitatory amino acids, which can produce lesions in the central nervous system. Additional examples of excitotoxins include, but are not limited to, sterol glucoside, including beta-sitosterol-beta-D-glucoside and cholesterol glucoside, methionine sulfoximine, and other substances known in the art to induce neuro-excitotoxic reactions in a patient. In one illustrative embodiment, the excitotoxin is a sterol glycoside. In further illustrative embodiments, the sterol glycoside is selected from the group consisting of beta-sitosterol-beta-D-glucoside and cholesterol glucoside, or analogs or derivatives thereof.

In embodiments, the invention may be used to treat neurological disease. As used herein, the term “neurological disease” or disorder relates to any disorder of the nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain, limitations in cognitive abilities and altered levels of consciousness. There are many recognized neurological disorders, some relatively common, but many rare. They may be assessed by neurological examination and studied and treated within the specialties of neurology and clinical neuropsychology.

Alzheimer's disease (AD), also referred to simply as Alzheimer's, is a chronic neurodegenerative disease that gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioral issues.

Parkinson's disease (PD), or simply Parkinson's, is a long-term degenerative disorder of the central nervous system that mainly affects the nerves in the basal ganglia that control movement. As the disease worsens, non-motor symptoms become more common. Early in the disease, the most obvious symptoms are shaking, rigidity, slowness of movement, and difficulty with walking. Thinking and behavioral problems may also occur. Dementia becomes common in the advanced stages of the disease. The main motor symptoms are collectively called “parkinsonism”, or a “parkinsonian syndrome”.

An example of a motor neuron disease is amyotrophic lateral sclerosis (ALS). ALS primarily involves the loss of spinal and cortical motor neurons, leading to increasing paralysis and eventually death. Early symptoms of ALS include but are not limited to, footdrop or weakness in a patient's legs, feet, or ankles, hand weakness or clumsiness, muscle cramps and twitching in the arms, shoulders, and tongue. ALS generally affects chewing, swallowing, speaking, and breathing, and eventually leads to paralysis of the muscles required to perform these functions. A review of various neurological diseases is set forth in Shaw et al., Neuroscience and Biobehavioral Reviews, 27:493 (2003), which is hereby incorporated by reference. The method and compositions of the present invention can be used for both human clinical medicine and veterinary medicine applications. The methods and compositions described herein may be used alone, or in combination with other methods or compositions.

Spinal muscular atrophy (SMA) is a rare neuromuscular disorder that results in the loss of motor neurons and progressive muscle wasting. It is usually diagnosed in infancy and if left untreated it is a common genetic cause of infant death in children. It may also appear later in life and then have a milder course. The common feature is progressive weakness of voluntary muscles, with arm, leg and respiratory muscles being affected first. Associated problems may include poor head control, difficulties swallowing, scoliosis, and joint contractures. Spinal muscular atrophy is due to an abnormality (mutation) in the SMN1 gene which encodes SMN, a protein necessary for survival of motor neurons. Loss of these neurons in the spinal cord prevents signaling between the brain and skeletal muscles. Another gene, SMN2, is considered a disease modifying gene, since usually the more the SMN2 copies, the milder is the disease course. The diagnosis of SMA is based on symptoms and confirmed by genetic testing.

In embodiments, the invention may be used to treat lysosomal storage diseases. The lysosomal storage diseases (LSDs) are a group of inherited metabolic disorders that are caused for the most part by enzyme deficiencies within the lysosome resulting in accumulation of undegraded substrate. This storage process leads to a broad spectrum of clinical manifestations depending on the specific substrate and site of accumulation. Examples of LSDs include the mucopolysaccharidoses, mucolipidoses, oligosaccharidoses, Pompe disease, Gaucher disease, Fabry disease, the Niemann-Pick disorders, and neuronal ceroid lipofuscinoses.

Gaucher disease is a rare inborn error of glycosphingolipid metabolism due to deficiency of lysosomal acid β-glucocerebrosidase (Gcase, “GBA”). Patients suffer from non-CNS symptoms and findings including hepatosplenomegaly, bone marrow insufficiency leading to pancytopenia, lung disorders and fibrosis, and bone defects. In addition, a significant number of patients suffer from neurological manifestations, including defective saccadic eye movements and gaze, seizures, cognitive deficits, developmental delay, and movement disorders including Parkinson's disease. In addition to Gaucher disease patients (who possess mutations in both chromosomal alleles of GBA1 gene), patients with mutations in only one allele of GBA1 are at highly increased risk of Parkinson's disease (PD). The severity of PD symptoms-which include gait difficulty, a tremor at rest, rigidity, and often depression, sleep difficulties, and cognitive decline-correlate with the degree of enzyme activity reduction. Thus, Gaucher disease patients have a severe course, whereas patients with a single mild mutation in GBA1 typically have a more benign course. Mutation carriers are also at high risk of other PD-related disorders, including Lewy Body Dementia, characterized by executive dysfunction, psychosis, and a PD-like movement disorder, and multi-system atrophy, with characteristic motor and cognitive impairments.

### FIGURES

The invention is further described by the figures. These are not intended to limit the scope of the invention.

### SHORT DESCRIPTION OF THE FIGURES

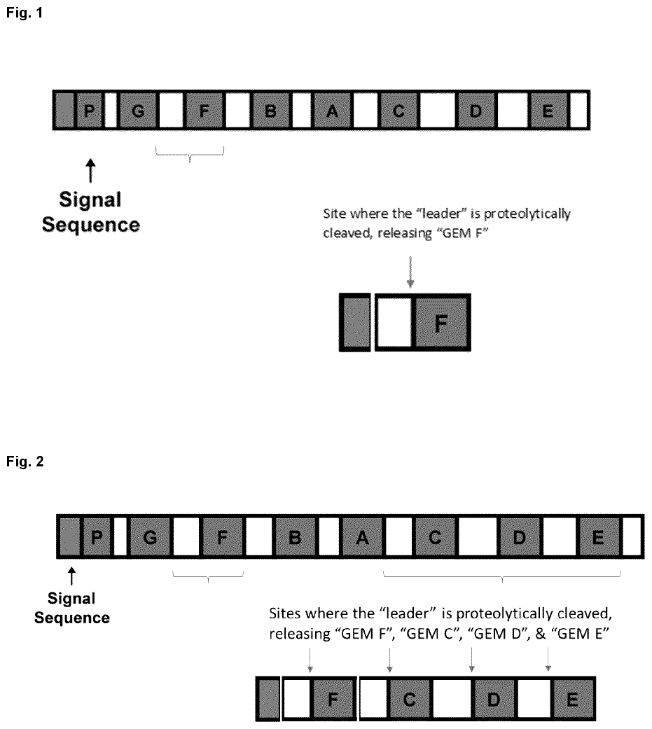

FIG. 1: AAV Single GEM Construct for the “Signal Sequence” attached to “leader+GEM F”.

FIG. 2: AAV Quad GEM Construct for the “Signal Sequence” attached to the “leader+GEM F”, “leader+GEM C”, “leader+GEM D”, & “leader+GEM E”.

FIG. 3: Vector Summary 1 of PGRN expressing pAAV.

FIG. 4: Vector Summary 2 of PGRN expressing pAAV.

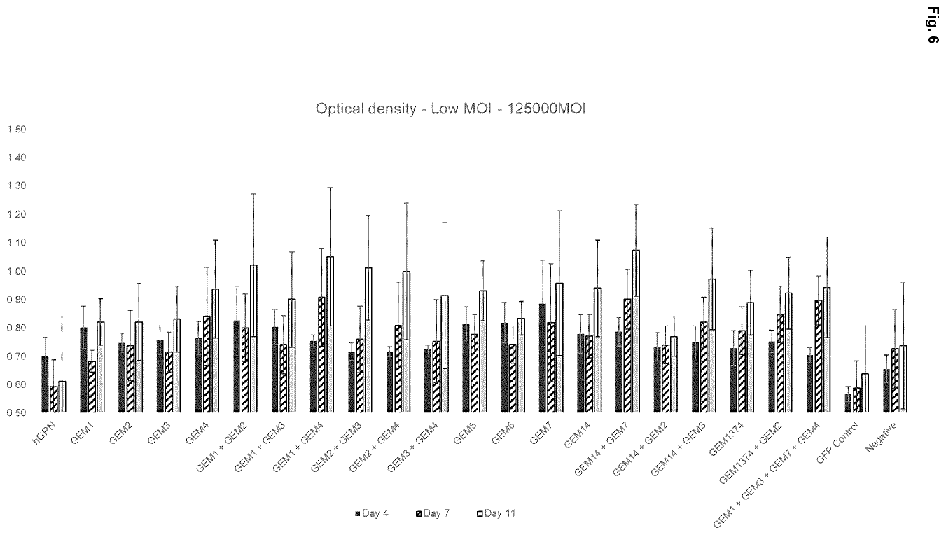

FIG. 5: Comparative cell proliferation of NSC34 cells after inoculation with different combinations of AAV vectors at 225000MOI.

FIG. 6: Comparative cell proliferation of NSC34 cells after inoculation with different combinations of AAV vectors at 125000MOI.

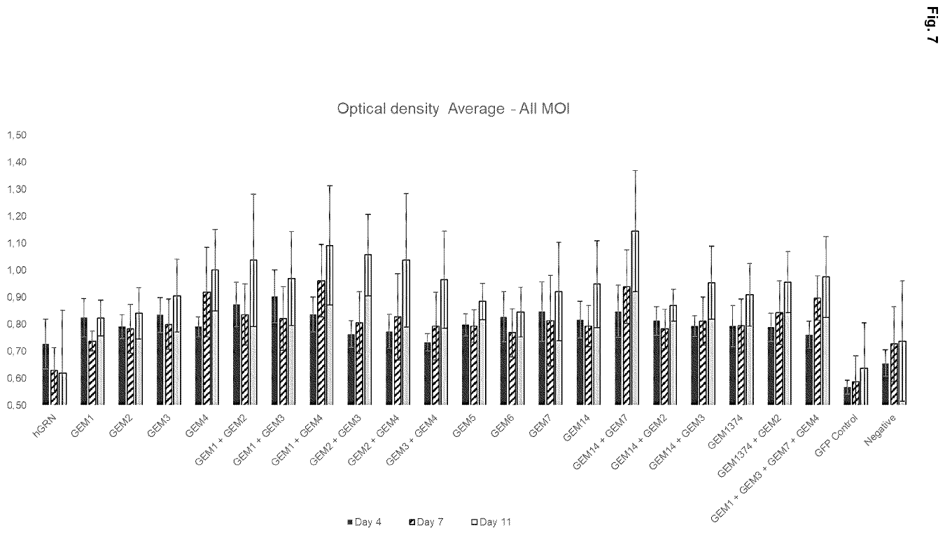

FIG. 7: Comparative cell proliferation of NSC34 cells after inoculation with different combinations of AAV vectors at 225000MOI and 125000MOI.

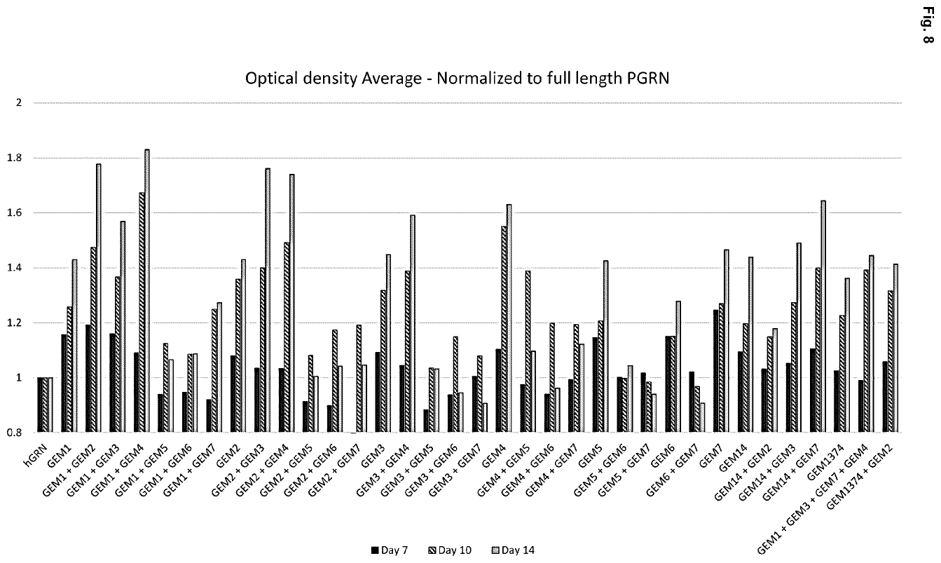

FIG. 8: Expression of hGRN protein modules (or their combination) in NSC34 cells promotes cell proliferation. Comparative cell proliferation of NSC34 cells after viral transduction with different combinations of AAV vectors.

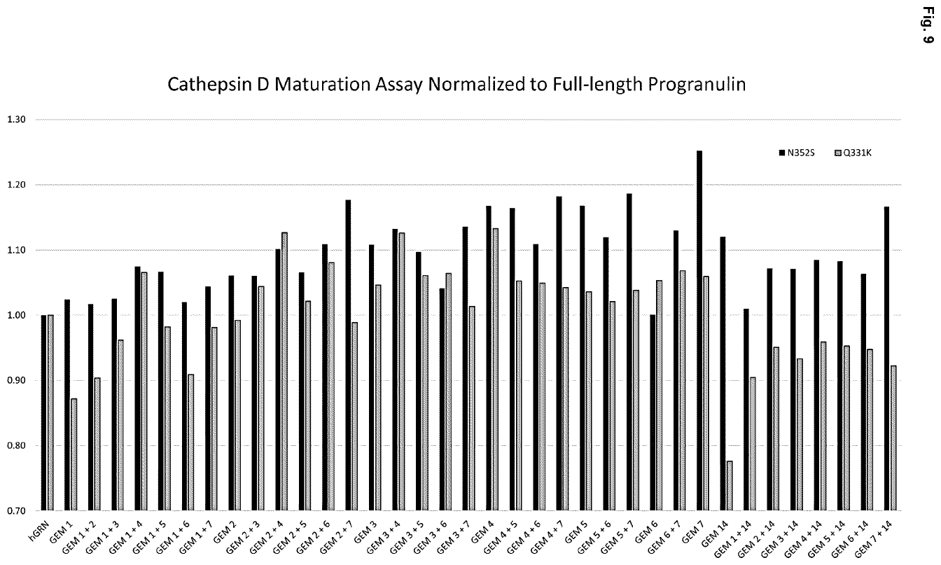

FIG. 9: Expression of hGRN protein modules (or their combination) in iPSC-derived motor neurons promotes cathepsin D maturation and activity.

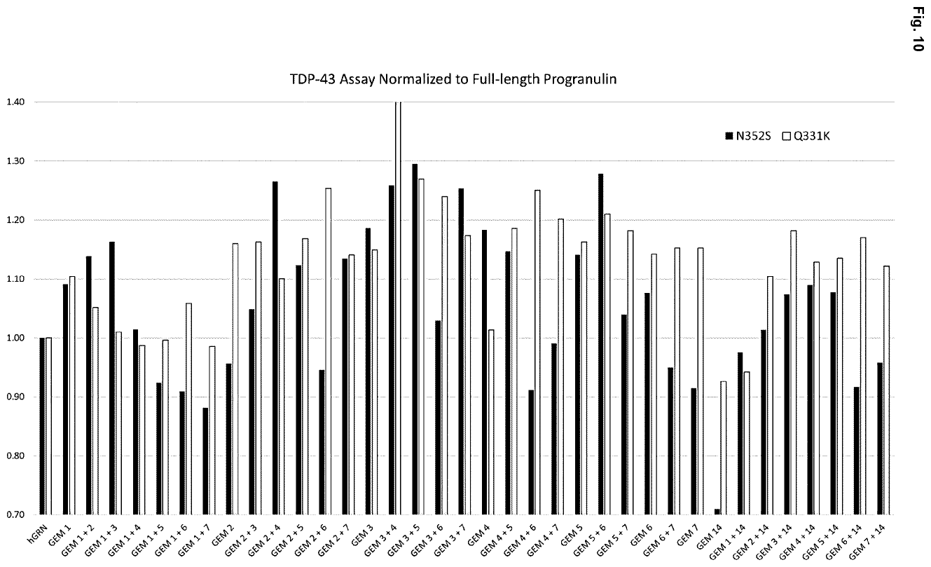

FIG. 10: Expression of hGRN protein modules (or their combination) in iPSC-derived motor neurons, appears to alleviate TDP-43 aggregation and accumulation.

FIG. 11: Survival of NSC-34 cells after stable genomic incorporation of mini-PGRNs.

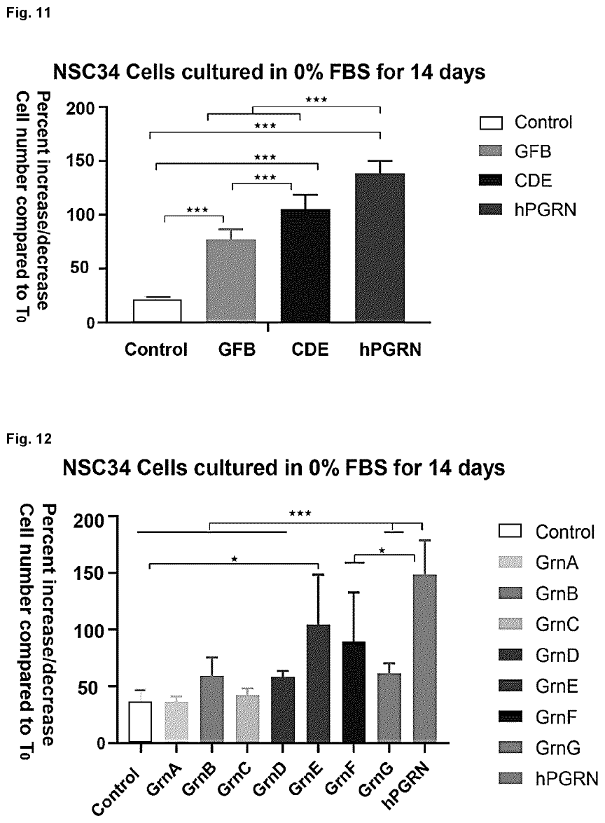

FIG. 12: Survival of NSC-34 cells after stable genomic incorporation of Grn modules (GEMs) GrnA, B, C, D, E and F or human PGRN.

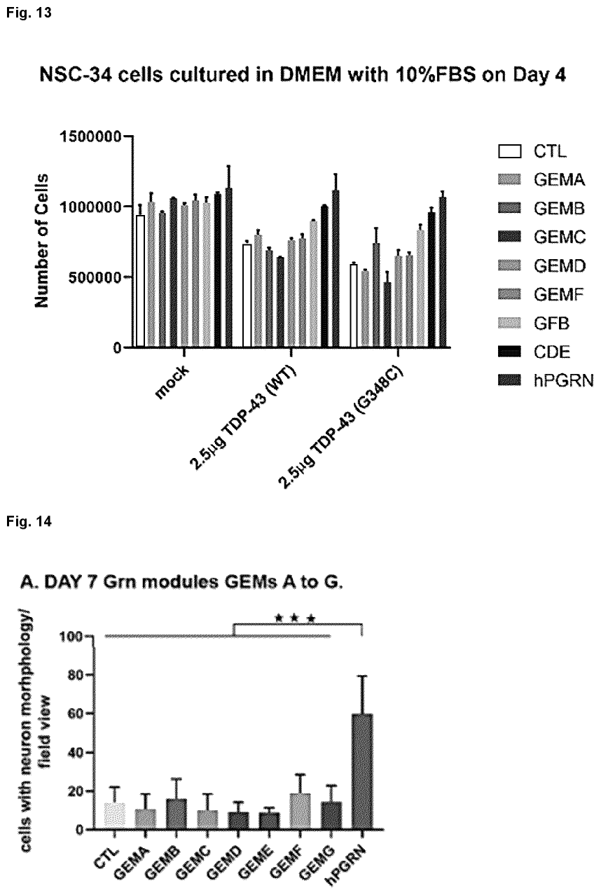

FIG. 13: The mini-PGRNs CDE and GFB show protective activity against the toxicity of the ALS related molecules TDP-43 and mutant TDP-43.

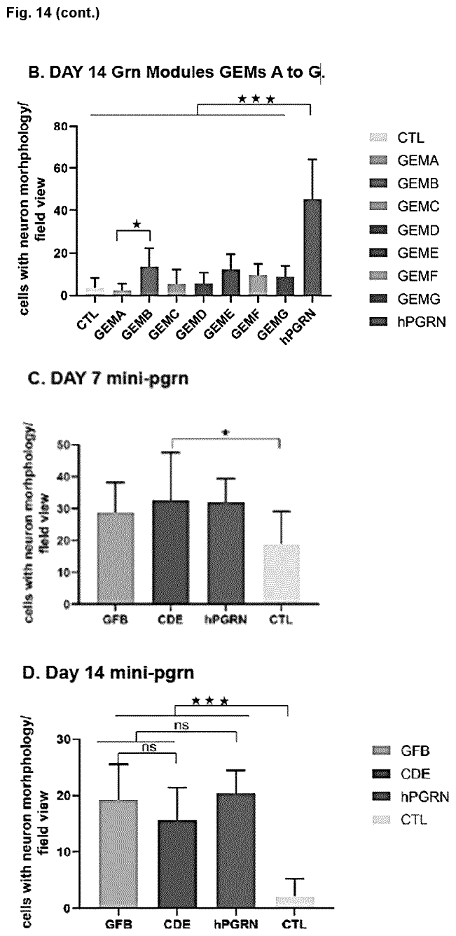

FIG. 14: The number of cells retaining a well-defined neuronal morphology was assessed after stress testing by serum deprivation.

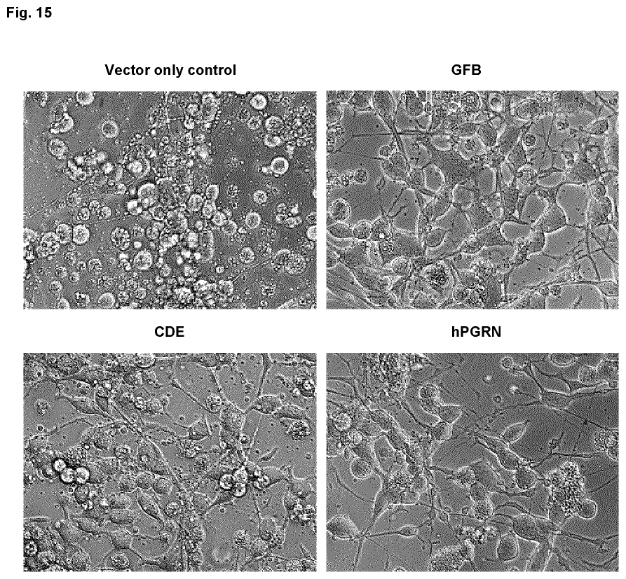

FIG. 15: Morphology of NSC-34 cells after stable genomic incorporation of half-PGRNs after 14 days of serum-withdrawal.

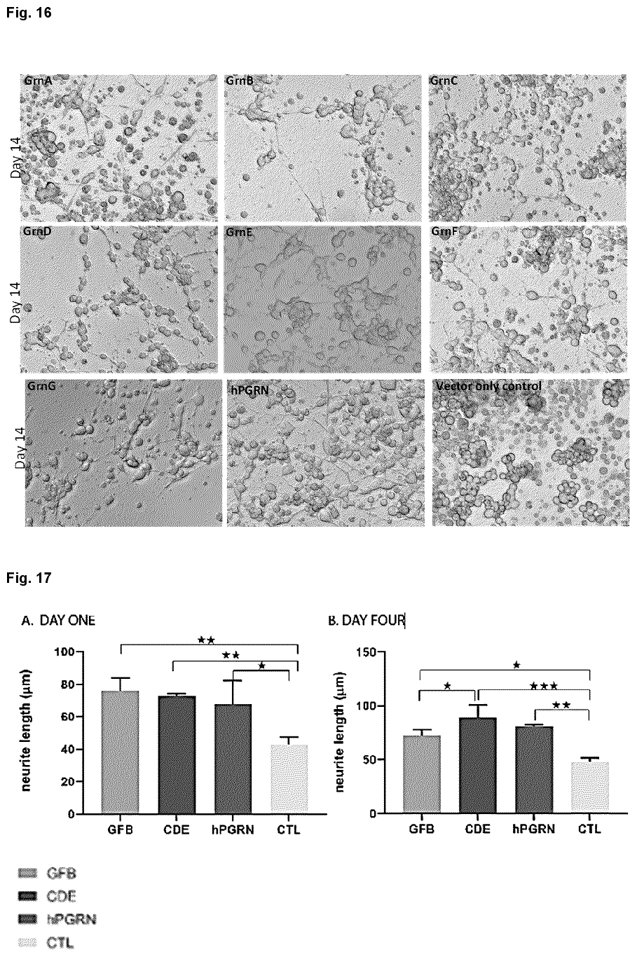

FIG. 16: Morphology of NSC-34 cells after stable genomic incorporation of individual Grn modules (GEMs) after 14 days of serum-withdrawal.

FIG. 17: The length of neurite-like extensions in NSC-34 control cells, hPGRN cells, and cells stably expressing the mini-PGRNs GFB and CDE.

### DETAILED DESCRIPTION OF THE FIGURES

FIG. 1: AAV Single GEM Construct for the “Signal Sequence” attached to the “leader+GEM F”. The Signal sequence is used to export the protein, natural to the full-length progranulin molecule, where it is naturally processed into the GEM subunits at the “end” of the “leader” peptide sequence.

FIG. 2: AAV Quad GEM Construct for the “Signal Sequence” attached to the “leader+GEM F”, “leader+GEM C”, “leader+GEM D”, & “leader+GEM E”. The Signal sequence is needed to export the protein, natural to the full-length progranulin molecule, where it is naturally processed into the GEM subunits at the “end” of the “leader” peptide sequence.

FIG. 3: Vector Summary 1 of PGRN expressing pAAV. Vector Summary and vector map of example pAAV vector pAAV [Exp]-CBh>hGRN[NM_002087.4]:WPRE, a mammalian gene expression AAV vector with the CBh promoter.

FIG. 4: Vector Summary 2 of PGRN expressing pAAV. Vector Summary and vector map of example pAAV vector pAAV [Exp]-Kan-CAG>hGRN[NM_002087.4]:WPRE3, a mammalian gene expression AAV vector with the CAG promoter.

FIG. 5: Comparative cell proliferation of NSC34 cells after inoculation with different combinations of AAV vectors. Cells were inoculated with different AAV construction at 225000MOI. The cellular proliferation was analysed by absorbance at 450 nM in microplate reader at 7, 10 and 14 days after inoculation with the AAV vectors. Data points represent the mean±SD for each condition for a single experiment performed in quadruplicate.

FIG. 6: Comparative cell proliferation of NSC34 cells after inoculation with different combinations of AAV vectors. Cells were inoculated with different AAV construction at 125000MOI. The cellular proliferation was analysed by absorbance at 450 nM in microplate reader at 7, 10 and 14 days after inoculation with the AAV vectors. Data points represent the mean±SD for each condition for a single experiment performed in quadruplicate.

FIG. 7: Comparative cell proliferation of NSC34 cells after inoculation with different combinations of AAV vectors. Cells were inoculated with different AAV construction at 125000MOI or 125000MOI. The cellular proliferation was analysed by absorbance at 450 nM in microplate reader at 7, 10 and 14 days after inoculation with the AAV vectors. Data points represent the mean±SD for both MOI conditions for a single experiment performed in quadruplicate.

FIG. 8: Expression of hGRN protein modules (or their combination) in NSC34 cells promotes cell proliferation. Comparative cell proliferation of NSC34 cells after inoculation with different combinations of AAV vectors. Data from FIG. 5-7 are combined and presented relative to levels of cell proliferation after treatment with full length PGRN.

FIG. 9: Expression of hGRN protein modules (or their combination) in iPSC-derived motor neurons promotes cathepsin D maturation and activity. Absorbances for GEM combinations were normalized against the full-length progranulin infected motor neurons and plotted.

FIG. 10: Expression of hGRN protein modules (or their combination) in iPSC-derived motor neurons, appears to alleviate TDP-43 aggregation and accumulation. Cellular concentrations of TDP-43 ranged between 10,000-25,000 μg/ml (Standard Range: 0-75,000 μg/ml). Absorbances for GEM combinations were normalized against the full-length progranulin infected motor neurons and plotted.

FIG. 11: Survival of NSC-34 cells after stable genomic incorporation of mini-PGRNs, corresponding to the amino-terminal half (GFB) and the carboxy-terminal half (CDE) of PGRN or full length human PGRN (hPGRN). Control cells were stably transfected with empty vector. Cell survival was challenged by incubation in medium containing 0% FBS. 100,000 cells were plated in each well (TO) and cell number counted after 14 days. (N=3, p<0.001-***, p<0.01-**, p<0.05-*, Error bars represent s.e.m.). The order of data bars in the graph from left to right on the x axis reflects the order of labels presented in the legend from top to bottom.

FIG. 12: Survival of NSC-34 cells after stable genomic incorporation of Grn modules (GEMs) GrnA, B, C, D, E and F or human PGRN (hPGRN). Control cells were stably transfected with empty vector. Cell survival was challenged by incubation in medium containing 0% FBS. 100,000 cells were plated in each well (To) and cell number counted after 14 days. (N=3, p<0.001-***, p<0.01-**, p<0.05-*, Error bars represent s.e.m.). The order of data bars in the graph from left to right on the x axis reflects the order of labels presented in the legend from top to bottom.

FIG. 13: The mini-PGRNs CDE and GFB show protective activity against the toxicity of the ALS related molecules TDP-43 and mutant TDP-43. NSC-34 cells stably expressing full grn modules or mini-PGRNs were plated in 12 well plates at 200000 cells per well containing DMEM with 10% FBS. After 24 hours the cells were transfected by lipofection with either full length wildtype TDP-43 or an ALS-causing mutant form of TDP-43 (G348C) both in pCS2+ at 2.5 ug each. The order of data bars in the graph from left to right on the x axis reflects the order of labels presented in the legend from top to bottom.

FIG. 14: The number of cells retaining a well-defined neuronal morphology after stress testing by serum deprivation: (A) Grn modules (GEMs) A, B, C, D, E, F and G seven days after serum withdrawal. (B) Grn modules (GEM) A, B, C, D, E, F and G fourteen days after serum withdrawal. (C) Mini-PGRNs GFB and CDE seven days after serum withdrawal (D) Mini-PGRNs fourteen days after serum withdrawal. The order of data bars in the graph from left to right on the x axis reflects the order of labels presented in the legend from top to bottom.

FIG. 15: Morphology of NSC-34 cells after stable genomic incorporation of half-PGRNs, corresponding to the amino-terminal half (GFB) or the carboxy-terminal half (CDE) of PGRN, after 14 days of serum-withdrawal. Note that in the empty vector only control almost all cells are showing evidence of apoptotic budding, detachment and rounding.

FIG. 16: Morphology of NSC-34 cells after stable genomic incorporation of individual Grn modules (GEMs) GrnA, GrnB, GrnC, GrnD, GrnE, GRNF and GrnG after 14 days of serum-withdrawal. Note that in the empty vector only control almost all cells are showing evidence of apoptotic budding, detachment and rounding.

FIG. 17: The length of neurite-like extensions in NSC-34 control cells, hPGRN cells, and cells stably expressing the mini-PGRNs GFB and CDE (A) one day after serum withdrawal and (B) four days after serum withdrawal. Note that the cells stably transfected with mini-PGRNs CDE and GFB show equivalent ability as to promote neurite-like extension as seen in cells stably transfected with hPGRN. The order of data bars in the graph from left to right on the x axis reflects the order of labels presented in the legend from top to bottom.

### Examples

The invention is further described by the following examples. The examples are intended to further describe the invention by way of practical example and do not represent a limiting description of the invention.

Progranulin is a secreted protein with important functions in several physiological and pathological processes, such as embryonic development, host defense, neuroprotection and wound repair. Structurally, progranulin consists of seven-and-a-half tandem repeats of the granulin/epithelin module (GEM), several of which have been isolated as discrete 6-kDa GEM peptides, also known as granulin polypeptides. All seven human GEMs can be expressed using recombinant approaches.

The present invention is based on beneficial granulins, preferably combinations of GEMs/granulins, that for example show improved effects over full length progranulin. The granulins and granulin combinations show beneficial properties in various in vitro models. Each of the granulins and/or combinations of granulins disclosed herein are to be tested in the models as follows. Preliminary investigation indicates beneficial biological effects, caused by the granulins and combinations thereof, as disclosed herein.

### Basic Methods:

Assays for assessing the effects of GEMs and combinations thereof are described below:

### NSC34 Cell Culture

The NSC34 cell line is maintained in DMEM with 10% fetal bovine serum unless otherwise stated [see Cashman et al., Dev Dyn. 194:209-21 (1992)]. For stable transfections NSC34 cells are transfected with human granulin (pcDNA-Pgrn) or empty vector (pcDNA) using Lipofectamine (Invitrogen) and selected with G418 for one month according to manufacture″s instructions. For example, serum deprivation assays are carried out in 6-well plates using 200,000 cells/well and cultured in 4 ml of RPMI (with glutamine) for 3, 6, 9, 12 and 15 days without the addition or exchange of fresh medium. For each time point the average cell number is determined over 6 visual fields per well at 10× magnification using an Olympus phase-contrast microscope.

As a further example, in hypoxia assays the cells are plated at a density of 50,000/well in 24-well plates, starved for 24 hours in RPMI without serum followed by the addition of fresh serum free RPMI or DMEM containing 5% serum and maintained in a hypoxia chamber containing 1% 02, 5% CO₂, balance N₂ for 72 hours. Cells are maintained in the hypoxic environment for 3 days, trypsinized and counted using a hemocytometer. For long term cultures NSC34 cells are plated at a density of 200,000/well in 6-well plates and maintained in serum free RPMI medium. Fresh medium was provided every 10 days and 10× magnification photos taken at 20 and 57 days using an Olympus phase-contrast microscope.

### NSC34 Cell Immunofluorescence

The NSC34 cell line, together with stable transfectants, are cultured on glass coverslips in DMEM with 10% fetal bovine serum. Cells are fixed in 4% PFA, rinsed twice with PBST, and incubated with permeabilization buffer (PBST with 0.2% Triton X-100) for 20 minutes. After being washed three times with PBST, the cultures are post-fixed for 10 minutes with 4% PFA, followed by extensive washing. Fixed cells are incubated in PBST with 0.5% (w/v) membrane blocking reagent (GE Healthcare) for one hour followed by the addition of sheep anti-mouse granulin, (1:500 dilution, R&D Systems).

Incubation with the primary antibody continued overnight at 40 C. Cultures are washed three times in PBST, then incubated with donkey anti-sheep Alexa-488 (1:200, Invitrogen) together with phalloidin-Alexa-594 conjugate (20 uM), in the blocking buffer for 45 minutes at room temperature. Cells are washed three times in PBST, then counterstained using 300 nM 4′,6-diamidino-2-phenylindole (DAPI) in PBS for 5 minutes at room temperature in the dark. Cultures are washed three times with PBST, twice with ddH₂O, and then mounted onto slides using Immu-mount (Thermo Fisher). Fluorescence is visualized with an Axioskop 2 microscope equipped with the appropriate fluorescence filters. Images were merged using Adobe Photoshop 7.0.

### Apoptosis Tunnel Assay

NSC34 cells are plated on German glass, photo-etched Coverslips (Electron Microscopy sciences) in 6-well plates at 200,000/well and cultured in 4 ml of RPMI (with glutamine) for six days. At time of fixation, cells are washed twice in PBS, then fixed using 4% PF A/PBS for 20 minutes. After being rinsed three times in PBST, cells are incubated in permeabilization buffer (0.2% Triton X-100 in PBST) for 20 minutes. Cells are subsequently post-fixed for 10 minutes with 4% PFA/PBS. After being washed extensively with PBST, cells are stored at 4° C. in sterile PBS.

At time of processing, cells are rinsed once with PBS, then overlaid with reaction solution from the Fluorescein In Situ Death Detection Kit (Roche Applied Science), as directed by manufacturer's instructions. Cells are incubated at 37° C. for 1 hour, and then rinsed twice with PBST at room temperature in the dark. After rinsing three times in PBST, cells are counterstained with 300 nm DAPI for 5 minutes in the dark. Cells are then rinsed twice with PBST and then mounted onto slides using Immumount (Thermo Fisher). Fluorescence was visualized with an Axioskop2 microscope equipped with appropriate filters and total cells (DAPI) versus apoptotic cells (FITC) are counted manually by visual inspection.

### Bromodeoxyuridine (BRDU) Proliferation Assay

NSC34 cells are plated on German glass, photo-etched Coverslips (Electron Microscopy Sciences) in 6-well plates at 200,000/well and cultured in 4 ml of RPMI (with glutamine) for six days. 12 hours prior to fixation/processing, BrdU labelling solution is added to each well at a concentration of 10 uM (Roche Applied Sciences). At the time of fixation, cells are washed three times in PBS to remove excess unincorporated BrdU, then fixed using 4% PFA/PBS for 20 minutes. After being rinsed three times in PBST, cells are incubated in permeabilization buffer (0.2% Triton X-100 in PBST) for 20 minutes. Cells are subsequently post-fixed for 10 minutes with 4% PFA/PBS. After being rinsed three times with PBST, the cells are placed in 0.1 M sodium borate pH 8.5 for 2 minutes at room temperature.

The cultures are incubated in PBST with 0.5% (w/v) membrane blocking reagent (GE Healthcare) for one hour followed by the addition of anti-BrdU Alexa-488 (1:200, Invitrogen) for 45 minutes in blocking buffer at room temperature After rinsing three times in PBST, cells are counterstained with 300 nm DAPI for 5 minutes in the dark. Cells are then rinsed twice with PBST, once with ddH2O and then mounted onto slides using Immu-mount (Thermo Fisher). Fluorescence is visualized with an Axioskop2 microscope equipped with appropriate filters and total cells (DAPI) versus proliferating cells (Alexa-488) were counted manually by visual inspection.

Specific examples using the apoptosis tunnel assay or bromodeoxyuridine (brdu) proliferation assay are not disclosed in the examples below, However, these methods represent potentially useful approaches to determining GEM or GEM combination function. The GEM combinations of the invention may therefore be assessed using these methods in order to show beneficial properties.

### Additional Methods

Additional in vitro methods suitable for assessing the effects of granulins are described in Cara L Ryan et al, Progranulin is expressed within motor neurons and promotes neuronal cell survival, BMC Neuroscience 2009, 10:130 doi: 10.1186/1471-2202-10-130.

For example, granulins provide sufficient trophic stimulus to maintain prolonged survival of NSC-34 cells in serum-free medium. Granulin expression can prevent apoptosis of NSC-34 cells induced by serum deprivation and exogenous granulins increase cell survival.

Further experimental approaches to be employed to show the effect of the granulin approach demonstrated herein are disclosed in Ederle and Dormann, FEBS Letters 591 (2017) 1489-1507.

Further experimental approaches to be employed to show the effect of the granulin approach demonstrated herein are disclosed in Beel et al. Molecular Neurodegeneration (2018) 13:55, https://doi.org/10.1186/s13024-018-0288-y.

Additional methods suitable for assessing the effects of granulins are described in Chitramuthu B P, Kay D G, Bateman A, Bennett H P J (2017) Neurotrophic effects of progranulin in vivo in reversing motor neuron defects caused by over or under expression of TDP-43 or FUS, pLoS ONE 12 (3): e0174784.

For example, mutations within the GRN gene cause frontotemporal lobar degeneration (FTLD). The affected neurons display distinctive TAR DNA binding protein 43 (TDP-43) inclusions. TDP-43 inclusions are also found in affected neurons of patients with other neurodegenerative diseases including amyotrophic lateral sclerosis (ALS) and Alzheimer's disease. In ALS, TDP-43 inclusions are typically also immunoreactive for fused in sarcoma (FUS). Mutations within TDP-43 or FUS are themselves neuropathogenic in ALS and some cases of FTLD. Analysis is therefore possible using the outgrowth of caudal primary motor neurons (MNs) in zebrafish embryos to investigate the interaction of PGRN with TDP-43 and FUS in vivo. As reported previously, depletion of zebrafish PGRNA (zfPGRN-A) is associated with truncated primary MNs and impaired motor function.

By way of example, the invention described herein is expected to or has been demonstrated to show one or more effects of:

Depletion of zfPGRN-A results in primary MNs outgrowth stalling at the horizontal myoseptum, a line of demarcation separating the myotome into dorsal and ventral compartments that is where the final destination of primary motor is assigned. Successful axonal outgrowth beyond the horizontal myoseptum depends in part upon formation of acetylcholine receptor clusters and this was found to be disorganized upon depletion of zfPGRN-A. Granulins are considered potentially effective to reverse the effects of zfPGRN-A knockdown. Both knockdown of TDP-43 or FUS, as well as expression of humanTDP-43 and FUS mutants results in MN abnormalities that are expected to be reversed by co-expression of granulins. The expected ability of granulin expression to override TDP-43 and FUS neurotoxicity due to partial loss of function or mutation in the corresponding genes is considered of therapeutic relevance.

The effect of granulin(s) on the neurodegenerative phenotype in TDP-43 (A315T) can be assessed. It is expected that granulin(s) reduce the levels of insoluble TDP-43 and histology of the spinal cord revealed a protective effect of granulin(s) on the loss of large axon fibers in the lateral horn, the most severely affected fiber pool in this mouse model. Overexpression of granulin(s) is expected to significantly slow down disease progression, extending the median survival by approximately 130 days. We expect granulin(s) to be effective in attenuating mutant TDP-43-induced neurodegeneration.

The following experimental examples represent beneficial effects of GEM treatment demonstrated through experimental approaches, employing administration of GEMs and GEM combinations according to the invention:

### Example 1: Proliferative Effect of GEMs on the NSC34 Cell Line

This example sets out to screen the proliferative effect of different progranulin (GRN) modules (GEMs, or combinations thereof) in the NSC34 cell line. The AAV-mediated progranulin gene modules (GRN) have been tested at 225000 and 125000 MOI and appropriate controls (hGRN full-length and GFP) have been included as well.

### Methods Summary:

NSC34 cells were seeded in 96-well plates at a density of 5.000 cells/well in presence of 225000 MOI or 125000 MOI of AAV-mediated progranulin gene modules or modules combination. Appropriate controls (hGRN, GFP and vehicle) were included as well. After 72 h of incubation, cell medium is replaced by DMEM with 1% FBS. The cell growth was determined on days 7, 10 and 14 post-infection using Cell Counting Kit-8 (CCK-8) method from Sigma Aldrich. This assay allows cell viability quantification using WST-8 reagent, which is bioreduced by cellular dehydrogenases to an orange formazan product that is soluble in tissue culture medium. The amount of formazan produced is directly proportional to the number of living cells. On day 14 post-infection, the cells were stained with Hoechst. Nuclei images and transmitted images were acquired with the Cell Insight High-Content Bioimager CX7 from Thermo Fisher. The experiments were carried out in quadruplicate.

### Materials and Methods:

The GEM nomenclature used in the present examples include an alternative numbering scheme, according to the following table:

| Granulin/GEM: | Number: |
|---|---|
| F | 1 |
| B | 2 |
| C | 3 |
| E | 4 |
| G | 5 |
| A | 6 |
| D | 7 |
| F + E | 14 |
| F + C + D + E | 1374 |

### Reagents and Equipment:

NSC34 provided by the inventor(s)
DMEM (Sigma-Aldrich D6429)
FBS (Sigma-Aldrich F2442, batch BCBW6329)
Flat bottom black 96-well plates (Becton Dickinson 353219, batchE1804340)
Cell Insight High-Content Bioimager CX7 from Thermofisher
Cell Counting Kit-8 (Sigma-Aldrich-96992)

### Virus Titer Employed (GC/Ml):

GFP: 1.26×10¹²
hGRN: 1.77×10¹²
GEM 1: 5.65×10¹¹
GEM 2: 7.25×10¹¹
GEM 3: 1.07×10¹²
GEM 4: 2.26×10¹¹
GEM 5: 3.52×10¹¹
GEM 6: 6.23×10¹¹
GEM 7: 2.96×10¹¹
GEM14: 1.04×10¹²
GEM1374: 9.35×10¹¹

The AAV-mediated progranulin gene modules were diluted 1/10 in DMEM medium supplemented with 10% FBS to obtain to obtain the following dilution factor corresponding to 225000 MOI and 125000MOI.

Actual Dilution Factors (uL); 1 to 10 Virus Stock Dilution/well

| | | |
|---|---|---|
| MOI | 225000 | 125000 |
| GFP | 9 | 0 |
| hGRN | 7 | 4 |
| GEM 1 | 20 | 12 |
| GEM 2 | 16 | 9 |
| GEM 3 | 11 | 6 |
| GEM 4 | 50 | 28 |
| GEM 5 | 32 | 18 |
| GEM 6 | 19 | 11 |
| GEM 7 | 39 | 22 |
| GEM14 | 11 | 7 |
| GEM1374 | 13 | 7 |

Recombinant NSC34 cell line was thawed (2×10⁶ cells per T75). Cells were maintained in DMEM supplemented with 10% FBS at 37° C. in a humidified 5% CO₂ atmosphere. Cells were plated in 96-well plates with a density of 5.000 cells per well in presence of 225000 MOI or 125000 MOI of AVV-hGRN modules or combination modules. Cells were maintained in DMEM medium supplemented with 10% FBS for 72 h at 37° C. in a humidified 5% CO₂ atmosphere. Each condition was carried out in quadruplicate.

96 h post-inoculation, the culture medium was removed from the wells and 10 μl of CCK-8 reagent (WST-8)+90 μl basal medium was added to each well and the plate was incubated at 37° C. After 1 hour, absorbance was measured at 450 nm using the Synergy II microplate reader (Biotek Instruments Inc., Winooski, United States). Then, WST-8 containing culture media was removed from the wells and replaced for 200 μl of the initial basal medium supplemented with 1% FBS.

10-day post-inoculation, the culture medium was removed from the wells and 10 μl of CCK-8 reagent (WST-8)+90 μl basal medium was added to each well and the plate was incubated at 37° C. After 1 hour, absorbance was measured at 450 nm using the Synergy II microplate reader (Biotek Instruments Inc., Winooski, United States). Then, WST-8 containing culture media was removed from the wells and replaced for 200 μl of the initial basal medium supplemented with 1% FBS.

14-day post-inoculation, the culture medium was removed from the wells and 10 μl of CCK-8 reagent (WST-8)+90 μl basal medium was added to each well and the plate was incubated at 37° C. After 1 hour, absorbance was measured at 450 nm using the Synergy II microplate reader (Biotek Instruments Inc., Winooski, United States). Then, the nuclei were stained using Hoechst (0.5 μg/ml) during 30 min and the fluorescence was measured using a Cell Insight High-Content Bioimager from Thermo Fisher. To detect the Hoechst, the filters used were 380/10 and 460/10 nm for excitation and emission, respectively. Additionally, transmittance images are taken for each well. The images were obtained with an objective of 20× taking 2 pictures of each well.

### Results:

Prior to the addition of cells, AVV Stocks for hGRN constructs were diluted into 1/10 (“Stock dilution” into DMEM with 10% serum. Following the Matrix (attached Methods), the appropriate volume of each Stock Dilution was added to the appropriate well to bring the MOI to 225000MOI or 125000MOI respectively. The immortalized motor neuron cell line NSC-34 was harvested by trypsinization, centrifuged at 1500 rpm for 5 minutes, then cells were resuspended in 1 ml of complete culture medium (DMEM 10% FBS). For measuring of cell viability, 10 μl of cell solution was stained with 10 μl of trypan blue dye and were counted using a hemocytometer. Cell solution was diluted to 5×10⁴ cell/ml density and 100 μl of cell suspension containing 5000 cells, was dispensed in each well of 96-well plates. After cell inoculation, the microtiter plates were incubated at 37° C., 5% CO₂, 95% air and 100% relative humidity for 72 hr. Then, culture medium was removed from the wells and replaced for 200 μl of DMEM supplemented with 1% of FBS. 10 days after infection, culture medium was removed from the wells and replaced for 200 μl of DMEM supplemented with 1.0% of FBS. The AAV-inoculated plates were monitored by microscopic observation and the cell proliferation was measured by WST8 at 4, 10 and 14 days.

The inoculation efficiency of the AAV vectors in this experiment was calculated by quantifying the percentage number of green cells (AAV-GFP) versus the number of total cells (total nuclei stained by Hoechst). The infection efficiency on plate A and plate B was 44.8% and 50.6% respectively.

Expression of hGRN protein modules (or their combination) in NSC34 cells promotes cell proliferation. The proliferation of NSC34 cells is more efficient in cells inoculated with 225000 MOI of AAV containing hGRN genetic material. The growth rate with respect to the negative control (GFP or non-inoculated cells) is more pronounced as the number of days in culture increases, with the largest difference occurring 14 days after inoculation.

The experimental results are shown in FIGS. 5 to 8 below. The results are the average of four independent replicates. For all plots: error bars represent: +/−S.D.

FIG. 5-8 demonstrate that combinations of the GEM modules showed an enhanced effect on the increase in NSC34 cell proliferation. By way of example GEMs F+E, F+B, F+C and B+C, B+E and C+E (1+4, 1+2, 1+3, 2+3, 2+4, 3+4 and 1+4+7) in addition to GEMs F+E+D+C (1+4+7+3) were beneficial in promoting NSC34 cell proliferation. Also beneficial are combinations 1+4+2, and 1+4+3.

As can be observed from FIG. 8, showing the NSC34 proliferation relative to full length PGRN, of note are the GEM combinations including GEM 1(E), with one or more of GEM2(B), 3(C) or 4(E), show improved performance over full length PGRN and in comparison to other GEMs individually or in combination.

Furthermore, the GEM combinations including GEM 2(B), with one or more of GEM3(C) or 4(E), show improved performance over full length PGRN and in comparison to other GEMs individually or in combination.

Furthermore, the GEM combinations including GEM 3(C), with GEM4(E), show improved performance over full length PGRN and in comparison to other GEMs individually or in combination.

Thus, unique combinations of GEMs, preferably those comprising double or triple GEM combinations of GEMs E, C, B and F, appear to support cell proliferation of NSC34 motor-neuron like cell line to a greater extent than treatment with full length PGRN or other GEMs alone or in combination.

### Example 2: Cathepsin D Maturation Effects of GEMs on Motor Neuron Cells with Known TDP-43 Mutations

This example sets out to screen the cathepsin D maturation potential of different progranulin (GRN) modules (GEMs, or combinations thereof) in motor neuron cell lines with known TDP-43 mutations. Cathepsins are lysosomal enzymes that are also used as sensitive markers in various toxicological investigations. The AAV-mediated progranulin gene modules (GRN) have been tested at a 250000 MOI and appropriate controls (hGRN full-length and GFP) have been included as well.

### Methods Summary:

Human Motor Neurons (iPSC-derived, heterozygous TDP-43 mutations at N352S or M337V) are derived from a genetically modified normal iPSC line carrying the heterozygous N352S or M337V mutation in the TDP-43 gene. iXCells™ hiPSC-derived motor neurons express typical markers of motor neurons, e.g., HB9 (MNX1), ISL1, CHAT, with the purity higher than 85%. Most of the cells will express high level of HB9 and ISL-1 after thawing, and after 5-7 days, will express high levels of CHAT and MAP2. Induced pluripotent stem cells, terminally differentiated into motor neurons, were seeded in 96-well plates at a density of 10,000 cells/well in presence of 250,000 MOI of AAV-mediated progranulin gene modules or module combinations. Appropriate controls (hGRN, GFP and vehicle) were included as well. After 7 days post-infection, the Cathepsin-D Activity assay from RayBiotech (Cat #: 68AT-CathD-S100) was used to determine if single GEM(s) or GEM combination(s) enhanced the efficiency of the TDP-43 mutant motor neurons to cleave the preferred cathepsin-D substrate sequence GKPILFFRLK (Dnp)-DR-NH2 labeled with MCA. This is quantified using a fluorometer or fluorescence plate reader at Ex/Em=328/460 nm.

### Materials and Methods:

The GEM nomenclature used in the present examples include an alternative numbering scheme, according to the following table:

| Granulin/GEM: | Number: |
|---|---|
| F | 1 |
| B | 2 |
| C | 3 |
| E | 4 |
| G | 5 |
| A | 6 |
| D | 7 |
| F + E | 14 |
| F + C + D + E | 1374 |

### Reagents and Equipment:

iXCell Human Motor Neurons (iPSC-derived, TDP-43 mutation, N352S, HET): 40HU-102-2M
iXCell Human Motor Neurons (iPSC-derived, TDP-43 mutation, Q331K, HET): 40HU-103-2M
iXCell Motor Neuron Maintenance Medium (Cat #MD-0022)
RayBiotech Cathepsin D Activity Assay Kit: 68AT-CathD-S100
Flat bottom black 96-well plates (Becton Dickinson 353219, batchE1804340)

### Virus Titer Employed (GC/Ml):

GFP: 1.26×10¹²
hGRN: 4.37×10¹³
GEM 1:5.65×10¹¹
GEM 2:7.25×10¹¹
GEM 3:1.07×10¹²
GEM 4:2.26×10¹¹
GEM 5:3.52×10¹¹
GEM 6:6.23×10¹¹
GEM 7:2.96×10¹¹
GEM14: 1.04×10¹²
GEM1374: 9.35×10¹¹

The AAV-mediated progranulin gene modules were diluted 1/10 in Motor Neuron Maintenance Medium to obtain to obtain the following dilution factor corresponding to 250000 MOI.

Actual Dilution Factors (uL): 1 to 10 Virus Stock Dilution/well

| | |
|---|---|
| MOI | 250000 |
| GFP | 20 |
| hGRN | 1 |
| GEM 1 | 45 |
| GEM 2 | 35 |
| GEM 3 | 24 |
| GEM 4 | 111 |
| GEM 5 | 72 |
| GEM 6 | 41 |
| GEM 7 | 85 |
| GEM14 | 25 |
| GEM1374 | 27 |

iPSC-derived motor neuron cells were thawed (1×106 cells per 96-well plate). Cells were maintained in Motor Neuron Maintenance Medium at 37° C. in a humidified 5% CO2 atmosphere. Cells were plated in 96-well plates with a density of 10.000 cells per well. After 24 hrs, the virus 1 to 10 dilutions were added to a final 250000 MOI AVV-hGRN modules or combination modules. Cells were maintained in Motor Neuron Maintenance Medium for 72 h at 37° C. in a humidified 5% CO2 atmosphere.

Five days post-inoculation, the culture medium was removed from the wells and 10 μl of CCK-8 reagent (WST-8)+90 μl basal medium was added to each well and the plate was incubated at 37° C. After 1 hour, absorbance was measured at 450 nm using the Synergy II microplate reader (Biotek Instruments Inc., Winooski, United States). Then, WST-8 containing culture media was removed from the wells and the Cathepsin D assay was performed.

### Assay Procedure:

1. Collect cells (1-2×10⁶) by centrifugation.
2. Lyse cells in 200 μl of chilled CD Cell Lysis Buffer. Incubate cells on ice for 10 min.
3. Centrifuge at top speed in a microcentrifuge for 5 min, transfer the supernatant to a new tube. Add 5-50 μl of cell lysate to a 96-well plate for each assay.
4. Bring up the volume to 50 μl of CD Reaction Buffer for each sample.
5. Prepare a master assay mix for each assay. Each assay needs: 50 μl of Reaction Buffer+2 μl CD substrate. Mix well.
6. Add 52 μl of master mixed into each assay well. Mix well
7. Incubate at 37° C. for 1-2 hours.
8. Read samples in a fluorometer equipped with a 328-nm excitation filter and 460-nm emission filter.

Fold-increase in Cathepsin D activity can be determined by comparing the relative fluorescence units (RFU) per million cells, or RFU per microgram of protein in a cell lysate sample, or RFU fold increase of treated versus untreated control or negative control samples.

### Results:

Absorbances for GEM combinations were normalized against the full-length progranulin infected motor neurons and plotted. Calculations above 1.00 are interpreted as GEM combinations which were able to increase the maturation of Cathepsin D, thus releasing more fluorescent substrate, more than in cells expressing the full-length progranulin. Calculations below 1.00 are interpreted as having no impact, or a negative impact, on a motor neuron's ability to promote the maturation of pro-Cathepsin D into fully mature and functional Cathepsin D protein.

Expression of hGRN protein modules (or their combination) in iPSC-derived motor neurons promotes cathepsin D maturation and activity. The Cathepsin D substrate cleavage rate with respect to full-length progranulin AAV infected cells shows variability from both GEM combinations and TDP-43 mutation.

Data is presented in FIG. 9.

As can be observed from FIG. 9, showing results from a Cathepsin D Maturation assay relative to full length PGRN, of note are the GEM combinations including 1+4, 1+5, 1+7 (F+E, D or G), which show improved performance over full length PGRN and in comparison to other GEMs individually or in combination.

Furthermore, the GEM combinations including GEM 2, with one or more of 3, 4, 5, 6 or 7, (GEM B, with C, E, G, A or D) show improved performance over full length PGRN and in comparison to other GEMs individually or in combination.

Furthermore, the GEM combinations including GEM 3, with one or more of 3, 4, 5, 6 or 7, (GEM C, with E, G, A or D) show improved performance over full length PGRN and in comparison to other GEMs individually or in combination.

Furthermore, the GEM combinations including GEM 4, with one or more of 5, 6 or 7, (GEM E, with G, A or D) show improved performance over full length PGRN and in comparison to other GEMs individually or in combination.

Furthermore, the GEM combinations including GEM 5, with one or more of 6 or 7, (GEM G, with A or D) show improved performance over full length PGRN and in comparison to other GEMs individually or in combination. GEMs 6+7 and 7 alone also showed improvements.

Under these experimental conditions, the combination of the peptide modules that showed the greatest improvements appear to be 2+7, 3+7, 4+7, 5+7, 6+7, and GEM 7 alone (B+D, C+D, E+D, G+D, A+D, and GEM D), which all showed enhanced Cathepsin D effects in the motor neuron cells. Also showing an enhanced effect on Cathepsin D maturation levels were combinations with GEM 4, 1+4 and 1+4+7 (GEM E combinations, and F+E+D).

### Example 3: Effects of GEMs on TDP43 Levels in Motor Neuron Cells with Known TDP43 Mutations

This example sets out to screen the potential of different progranulin (GRN) modules (GEMs, or combinations thereof) to alter/reduce the amount of TDP43 protein accumulation in motor neuron cell lines with known TDP43 mutations. Human TDP43 is an RNA-binding protein that is involved in various steps of RNA biogenesis and processing. Aberrant RNA processing, cellular compartmentalization, and protein degradation are associated with mutations in the TDP43 protein, and have a correlation with various neurological diseases. The AAV-mediated progranulin gene modules (GRN) have been tested at a 250,000 MOI and appropriate controls (hGRN full-length and GFP) have been included as well.

### Methods Summary:

Human Motor Neurons (iPSC-derived, heterozygous TDP-43 mutations at N352S or M337V) are derived from a genetically modified normal iPSC line carrying the heterozygous N352S or M337V mutation in the TDP-43 gene. iXCells™ hiPSC-derived motor neurons express typical markers of motor neurons, e.g., HB9 (MNX1), ISL1, CHAT, with the purity higher than 85%. Most of the cells will express high level of HB9 and ISL-1 after thawing, and after 5-7 days, will express high levels of CHAT and MAP2. Induced pluripotent stem cells, terminally differentiated into motor neurons, were seeded in 96-well plates at a density of 10,000 cells/well in presence of 250,000 MOI of AAV-mediated progranulin gene modules or module combinations. Appropriate controls (hGRN, GFP and vehicle) were included as well. After 7 days post-infection, the Cathepsin-D Activity assay from RayBiotech (Cat #: 68AT-CathD-S100) was used to determine if single GEM(s) or GEM combination(s) enhanced the efficiency of the TDP-43 mutant motor neurons to cleave the preferred cathepsin-D substrate sequence GKPILFFRLK (Dnp)-DR-NH2 labeled with MCA. This is quantified using a fluorometer or fluorescence plate reader at Ex/Em=328/460 nm.

### Materials and Methods:

| Granulin/GEM: | Number: |
|---|---|
| F | 1 |
| B | 2 |
| C | 3 |
| E | 4 |
| G | 5 |
| A | 6 |
| D | 7 |
| F + E | 14 |
| F + C + D + E | 1374 |

### Reagents and Equipment:

iXCell Human Motor Neurons (iPSC-derived, TDP-43 mutation, N352S, HET): 40HU-102-2M
iXCell Human Motor Neurons (iPSC-derived, TDP-43 mutation, Q331K, HET): 40HU-103-2M
iXCell Motor Neuron Maintenance Medium (Cat #MD-0022)
Abcam Human TDP43 SimpleStep ELISA Kit (TARDBP): ab282880
Flat bottom black 96-well plates (Becton Dickinson 353219, batchE1804340)

### Virus Titer Employed (GC/Ml):

GFP: 1.26×10¹²
hGRN: 4.37×10¹³
GEM 1:5.65×10¹¹
GEM 2:7.25×10¹¹
GEM 3:1.07×10¹²
GEM 4:2.26×10¹¹
GEM 5:3.52×10¹¹
GEM 6:6.23×10¹¹
GEM 7:2.96×10¹¹
GEM14: 1.04×10¹²
GEM1374: 9.35×10¹¹

The AAV-mediated progranulin gene modules were diluted 1/10 in Motor Neuron Maintenance Medium to obtain to obtain the following dilution factor corresponding to 250000 MOI.

Actual Dilution Factors (uL): 1 to 10 Virus Stock Dilution/well

| | |
|---|---|
| MOI | 250000 |
| GFP | 20 |
| hGRN | 1 |
| GEM 1 | 45 |
| GEM 2 | 35 |
| GEM 3 | 24 |
| GEM 4 | 111 |
| GEM 5 | 72 |
| GEM 6 | 41 |
| GEM 7 | 85 |
| GEM14 | 25 |
| GEM1374 | 27 |

iPSC-derived motor neuron cells were thawed (1×106 cells per 96-well plate). Cells were maintained in Motor Neuron Maintenance Medium at 37° C. in a humidified 5% CO2 atmosphere. Cells were plated in 96-well plates with a density of 10.000 cells per well. After 24 hrs, the virus 1 to 10 dilutions were added to a final 250000 MOI AVV-hGRN modules or combination modules. Cells were maintained in Motor Neuron Maintenance Medium for 72 h at 37° C. in a humidified 5% CO2 atmosphere.

Five days post-inoculation, the culture medium was removed from the wells and 10 μl of CCK-8 reagent (WST-8)+90 μl basal medium was added to each well and the plate was incubated at 37° C. After 1 hour, absorbance was measured at 450 nm using the Synergy II microplate reader (Biotek Instruments Inc., Winooski, United States). Then, WST-8 containing culture media was removed from the wells and the Cathepsin D assay was performed.

### Assay Procedure:

1. Prepare all reagents, cell samples, and standards as instructed
2. Add 50 UL standard or sample to appropriate wells
3. Add 50 μL Antibody Cocktail to all wells
4. Incubate at room temperature for 1 hour
5. Aspirate and wash each well three times with 350 μL 1× Wash Buffer PT
6. Add 100 μL TMB Development Solution to each well and incubate for 10 minutes.
7. Add 100 μL Stop Solution and read OD at 450 nm

### Results:

Cellular concentrations of TDP-43 ranged between 10,000-25,000 pg/ml (Standard Range: 0-75,000 pg/ml). Absorbances for GEM combinations were normalized against the full-length progranulin infected motor neurons and plotted. Calculations below 1.00 are interpreted as GEM combinations which were able to lower the TDP-43 concentrations more than cells expressing the full-length progranulin. Calculations above 1.00 are interpreted as having no impact, or a negative impact, on a motor neuron's ability to process and resolve TDP-43 proteinopathy.

Expression of hGRN protein modules (or their combination) in iPSC-derived motor neurons, appears to alleviate TDP-43 aggregation and accumulation. The TDP-43 accumulation rate with respect to full-length progranulin shows some variability from both GEM combinations and TDP-43 mutation.

Data is presented in FIG. 10.

As can be observed from FIG. 10, showing results for TDP-43 accumulation relative to full length PGRN, of note are the GEM combinations including 1+4, 1+5, 1+6, 1+7 (F+E, D, A or G), which show improved performance over full length PGRN and in comparison to other GEMs individually or in combination.

Furthermore, the GEM combinations including GEM 2 with 6 (GEM B, with A) showed improved performance over full length PGRN and in comparison to other GEMs individually or in combination.

Under these experimental conditions, the combination of the peptide modules 1+4, 1+5, 1+6 and 1+7 (F+E, F+A, and F+D) appeared to show the greatest enhanced effect on the ability of motor neuron cells to properly clear TDP-43, even with a known TDP-43 mutation. Also showing an enhanced effect on reducing TDP-43 protein levels were 1+4+6 and 1+4+7 (F+E+A and F+E+D).

### Example 4: Progranulin Expression Levels after GEM Treatment in Motor Neuron Cells with Known TDP-43 Mutations

This example seeks to screen the potential of different progranulin (GRN) modules (GEMs, or combinations thereof) in motor neuron cell lines with known TDP-43 mutations for any effects on the total expression of full-length progranulin. The AAV-mediated progranulin gene modules (GRN) are tested at a 250,000 MOI and appropriate controls (hGRN full-length and GFP) are included.

### Methods Summary:

Human Motor Neurons (iPSC-derived, heterozygous TDP-43 mutations at N352S or M337V) are derived from a genetically modified normal iPSC line carrying the heterozygous N352S or M337V mutation in the TDP-43 gene. iXCells™ hiPSC-derived motor neurons express typical markers of motor neurons, e.g., HB9 (MNX1), ISL1, CHAT, with the purity higher than 85%. Most of the cells will express high level of HB9 and ISL-1 after thawing, and after 5-7 days, will express high levels of CHAT and MAP2. Induced pluripotent stem cells, terminally differentiated into motor neurons, are seeded in 96-well plates at a density of 10,000 cells/well in presence of 250,000 MOI of AAV-mediated progranulin gene modules or module combinations. Appropriate controls (hGRN, GFP and vehicle) are included as well.

### Assay Procedure:

The assay is a sandwich Enzyme Linked-Immunosorbent Assay (ELISA) for quantitative determination of human progranulin in biological fluids. A polyclonal antibody specific for progranulin is precoated onto a 96-well microtiter plate. Standards and samples are pipetted into the wells for binding to the coated antibody. After extensive washing to remove unbound compounds, progranulin is recognized by the addition of a biotinylated polyclonal antibody specific for progranulin (Detection Antibody). After removal of excess biotinylated antibody, HRP labeled streptavidin (STREP-HRP) is added. Following a final washing, peroxidase activity is quantified using the substrate 3,3′,5,5′-tetramethylbenzidine (TMB). The intensity of the color reaction is measured at 450 nm after acidification and is directly proportional to the concentration of progranulin in the samples.

A strong signal is expected in full-length progranulin AAV wells. The inventor(s) postulates that expression of hGRN protein modules (GEMs or their combination) may lead to enhanced PGRN levels in treated wells. The GEMs (or their combination) may “free up” full-length progranulin, thus enabling a stronger signal, as the GEMs are performing a function that was initially required by the processing of endogenous full-length progranulin.

### Example 5: Effects of GEMs on Neuroinflammation

Elevated neuroinflammation is a pathological hallmark of neurodegenerative diseases. Full length PGRN is known to exhibit anti-neuroinflammatory properties. Conversely, exaggerated neuroinflammation is observed in PGRN deficient animals, where increased levels of activated astrocytes and microglial cells are observed with aging. This example seeks to screen the potential of different progranulin (GRN) modules (GEMs, or combinations thereof) on anti-neuroinflammatory effects, as observed for full length progranulin.

Lead candidate individual or combinations of GEMs, for example selected from the assessment of (i) cell survival assays, (ii) assays promoting mature neuronal phenotype, and/or (iii) assays measuring the reduction of toxic TDP-43, are evaluated for their ability to influence the production of pro- and anti-inflammatory cytokines in a human microglial cell line. The candidate GEM/GEM combinations effects on cytokine production are assessed under two conditions of culture, in the (i) absence and (ii) presence of lipopolysaccharide (LPS) activation.

### Methods Summary:

The human microglial cell line HMC3 (ATCC, CRL-3304) us infected with AAV-9 viral vectors encoding candidate individual or combination of GEMs. Three days following infection cells are subcultured and either exposed or not to LPS stimulation (0.5 mg/ml for 24 hours) to activate the microglial cells. Twenty-four hours later cell culture supernatants are analyzed for their content of a panel of pro and anti-inflammatory cytokines using the V-PLEX Proinflammatory Panel 1 Human Kit (Mesoscale K15049D; alternative kits may be employed). This kit permits quantitation of an array of both pro- and anti-inflammatory cytokines. Thus, IFN-γ, IL-1B, IL-2, IL-4, IL-6, IL-8, IL-10, IL-12 p70, IL-13, TNF-α can be quantitated.

The candidate GEM/GEM combinations effects on cytokine production are compared to that observed in cell culture supernatants from HMC3 cells expressing either full length PGRN (AAV-9 PGRN infected) acting as positive control, or GFP expressing & uninfected HMC3 cells. as negative controls.

The inventor(s) postulates that expression of hGRN protein modules (GEMs or their combination) exhibit beneficial anti-inflammatory effects, either at a level similar to or improved over treatment with full length PGRN.

### Example 6: Survival of NSC-34 Cells and Maintenance of Neuronal Morphology Upon Serum-Deprivation Stress, after Stable Genomic Incorporation of GEMs, Mini-PGRNs, and Full Length Human PGRN (hPGRN)

This example sets out to screen the survival enhancing effect of stable genomic integration of different progranulin (GRN) modules (GEMs, or combinations thereof), or mini-PGRNs, corresponding to the amino-terminal half (GFB) or the carboxy-terminal half (CDE) of full length human PGRN (hPGRN), in the NSC34 cell line.

NSC-34 cells stably expressing human progranulin (hPGRN), mini-PGRNs containing the N-terminal granulin modules GFB or the C-terminal granulin modules CDE, or individual GRN modules A, B, C, D, E, F and G were assessed for
(A): Survival upon serum-deprivation stress,
(B): Survival upon expression of the ALS-related molecules TDP-43 wild-type and mutant TDP-43 (G348C),
(D): Maintenance of neuronal morphology upon serum-deprivation stress, and
(C): Rates of neurite extension upon serum-withdrawal stress.

### Methods:

### Vectors:

Individual GEMs, and mini-PGRN CDE and GFB were inserted into (hPGRN-SP-pcDNA3.1/V5-His TOPO), a modified pcDNA3.1/V %-His TOPO designed to include the human PGRN secretory signal sequence (A. Bateman and B Chitramuthu). Plasmids were selected, purified and sequences to ensure fidelity of the sequence, to confirm that the insert was in frame and in the correct orientation to insert the hPGRN secretory signal peptide amino-terminally of the final protein product. The secretory signal peptide enables proteins to be secreted and to be routed to lysosomes.

### Cell Culture, Transfection and Validation:

NSC-34 cells were used as previously described. NSC-34 cells were maintained in DMEM with 10% fetal bovine serum (FBS). Stable transfectants that express GEMs grnA, grnB, grnC, grnD, grnE, grnF, grnG, mini-pgrn CDE and mini-pgrn GFB were generated by transfection with GEMs (in sp-pcDNA3.1 V5-Topo-GEMs) or sp-pcDNA3.1 V5-Topo for empty vector control transfections. Cells were transfected using Lipofectamine (Invitrogen) and selected with G418 (400 μg/ml) for 4 to 6 weeks according to manufacturer's instructions. To prevent phenotypic drift, stocks of the original transfectants were frozen in liquid N₂ and reanimated at regular intervals. Expression was confirmed by RT-PCR using total RNA isolated using Trizol reagent (Invitrogen). cDNA synthesis was performed with Revert-aid reverse transcriptase (Thermo Scientific). GEMs specific primer sets were designed used to amplify specific products.

### Cell Survival Bioassay:

NSC 34 cells expressing full grn modules or mini-PGRNs were plated in 6 well plates at 100000 cells per well containing DMEM with 10% FBS. After 24 hours, the media was replaced with DMEM containing 0% FBS. Cells were trypsinized and number of cells were counted at day 14 using the trypan blue dye exclusion assay to distinguish live versus dead cells. Statistical significance among experimental groups was determined by one-way ANOVA followed by Tukey's Multiple Comparisons Test (p<0.001-***, p<0.01-**, p<0.05-*) using GraphPad software (GraphPad Prism Software Inc., San Diego, CA) Error bars represent s.e.m.

### Cell Survival Bioassay-TDP-43 Challenge:

NSC-34 cells stably expressing full grn modules or mini-PGRNs were plated in 12 well plates at 200000 cells per well containing DMEM with 10% FBS. After 24 hours the cells were transfected by lipofection with either full length wildtype TDP-43 or an ALS-causing mutant form of TDP-43 (G348C) both in pCS2+ at 2.5 ug each. Control cells were mock transfected with reagents lacking DNA. Cells were propagated in 10% serum for four days, trypsinized, and viable cell number assessed by the trypan blue exclusion test.

### Morphology:

NSC-34 cells expressing full grn modules or mini-PGRNs were plated in 12 well plates at 100000 cells per well containing DMEM with 10% FBS. After 24 hours, the media was replaced with DMEM containing 0% FBS. To assess the maintenance of cells with neuronal morphology under stress conditions the cultures were monitored at 7 and 14 days in serum free medium, and photographs were acquired using an inverted phase contrast microscope. Neuronal morphology was assessed as spread cell body (not round cells) with clearly visible neuritic extension at least twice length of the cell body along its longest axis.

To evaluate the rate of neuritic extension we tested the two best performing constructs from above and assessed their average neurite extension length after one and four days in serum free medium compared to CTL cells stably transfected with an empty vector and hPGRN expressing cells. Cells were photographed in an inverted microscope and extension length was assessed using the ImageJ program in the FIJI open-sourced image processing package.

### Results:

### A. Serum-Deprivation Stress Challenge:

Survival of NSC-34 cells was assessed after stable genomic incorporation of mini-PGRNs, corresponding to the amino-terminal half (GFB) and the carboxy-terminal half (CDE) of PGRN or full length human PGRN (hPGRN). Control cells were stably transfected with empty vector. Cell survival was challenged by incubation in medium containing 0% FBS. 100,000 cells were plated in each well (TO) and cell number counted after 14 days.

As can be seen in FIG. 11, the mini-PGRNs GFB and CDE provide protection against serum-deprivation stress close to that provided by full length hPGRN. Since both the N-terminal mini-PGRN GFB and the C-terminal mini-PGRN CDE provide protection it is clear that activity is not confined to a single locus along full-length PGRN but is distributed between the N-terminal and C-terminal sections of PGRN.

As can be seen in FIG. 12, among the Grn modules (GEMs), E and F provide the strongest protection, suggesting that the protective activity detected in CDE may be centered on the E module and the protective activity of GFB may be centered on module F. Individual modules provide less protection than GEM combinations, as used in this experiment in the form of mini-PGRNs (see FIG. 11 above) suggesting that the activity of E and F is augmented by the presence of the other (potentially less active) modules (G, F in GFB and D, E in CDE) in the mini-PGRNs.

### B. Survival: TDP-43 Cell Toxicity Challenge:

NSC-34 cells stably expressing full grn modules or mini-PGRNs were plated in 12 well plates at 200000 cells per well containing DMEM with 10% FBS. After 24 hours the cells were transfected by lipofection with either full length wildtype TDP-43 or an ALS-causing mutant form of TDP-43 (G348C) both in pCS2+ at 2.5 ug each.

As can be seen in FIG. 13, CDE and GFB mini-PGRNs show protection against the ALS-related TDP-43 toxicity for both WT (i.e., sporadic ALS) and G348C (mutational ALS) that is close to that provided by full length hPGRN. The mini-PGRNs CDE and GFB show protective activity against the toxicity of the ALS related molecules TDP-43 and mutant TDP-43.

### C. Maintenance of Neuronal Morphology Upon Serum-Deprivation Stress:

The number of cells retaining a well-defined neuronal morphology was assessed after stress testing by serum deprivation. As can be seen from FIG. 14, the individual Grn modules (GEMs) G, F, B and E show a trend to improved morphology maintenance (panel B). Both of the mini-PGRNs GFB and CDE show improved maintenance of neuronal morphology after fourteen days of serum-deprivation stress that is indistinguishable from hPGRN.

Additional data is presented in FIGS. 15 and 16, demonstrating the morphology of NSC-34 cells after stable genomic incorporation of half-PGRNs, after 14 days of serum-withdrawal, and morphology of NSC-34 cells after stable genomic incorporation of individual Grn modules (GEMs) GrnA, GrnB, GrnC, GrnD, GrnE, GrnF and GrnG after 14 days of serum-withdrawal.

### D. Rate of Neurite Extension:

The best performing constructs from A through to C were tested for their ability to promote neurite-like extension compared to empty vector NSC-43 Control cells and NSC-34 hPGRN expressing cells in serum depleted medium. Assays were taken on day one and day four, both of which are before the onset of extensive apoptosis in CTL cells.

As can be seen in FIG. 17, the cells stably transfected with mini-PGRNs CDE and GFB show equivalent ability as to promote neurite-like extension as seen in cells stably transfected with hPGRN.

Additional GEMs and GEM combinations of the invention are being tested in the experiments of Examples 1-6. The inventor(s) postulate the effects observed in the examples disclosed herein may be reproduced for the same and additional GEM combinations of the invention.

1. A recombinant polypeptide or combination of multiple recombinant polypeptides, comprising two to six granulin/epithelin modules (GEMs).

2. The recombinant polypeptide or combination according to claim 1, comprising two to six granulin/epithelin modules (GEMs), comprising GEM E, and additionally one or more of GEM F, GEM B, GEM C and GEM D.

3. The recombinant polypeptide or combination according to claim 1, comprising granulin/epithelin module (GEM) E, and additionally GEM F.

4. The recombinant polypeptide or combination according to claim 1, comprising granulin/epithelin module (GEM) E, and additionally GEM C.

5. The recombinant polypeptide or combination according to claim 1, comprising granulin/epithelin module (GEM) E, and additionally GEM B.

6. The recombinant polypeptide or combination according to claim 1, comprising granulin/epithelin module (GEM) E, and additionally GEM F and GEM D.

7. The recombinant polypeptide or combination according to claim 1, comprising granulin/epithelin module (GEM) E, and additionally GEM F, GEM D and GEM C.

8. The recombinant polypeptide or combination according to claim 1, comprising two to six granulin/epithelin modules (GEMs), comprising GEM F, and additionally one or more of GEM A, GEM B, GEM E, GEM C and GEM D.

9. The recombinant polypeptide or combination according to claim 1, comprising granulin/epithelin module (GEM) F, and additionally GEM B.

10. The recombinant polypeptide or combination according to claim 1, comprising granulin/epithelin module (GEM) F, and additionally GEM A.

11. The recombinant polypeptide or combination according to claim 1, comprising granulin/epithelin module (GEM) F, and additionally GEM D.

12. The recombinant polypeptide or combination according to claim 1, comprising a signal sequence positioned N-terminally of the GEMs.

13. The recombinant polypeptide or combination according to claim 1, comprising one or more linker (leader) sequences positioned N-terminally of and/or between the GEMs.

14. The recombinant polypeptide or combination according to claim 1, wherein the polypeptide comprises or consists of a mixed portion of a full-length PGRN sequence according to SEQ ID NO 1, and/or does not consist of the full-length PGRN sequence according to SEQ ID NO 1.

15. The recombinant polypeptide or combination according to claim 1, wherein:
a. GEM E comprises or consists of SEQ ID NO 5,
b. GEM F comprises or consists of SEQ ID NO 2,
c. GEM C comprises or consists of SEQ ID NO 4,
d. GEM D comprises or consists of SEQ ID NO 8,
e. GEM A comprises or consists of SEQ ID NO 7, and/or
f. GEM B comprises or consists of SEQ ID NO 3.

16. The recombinant polypeptide or combination according to claim 1,
a. comprising a signal sequence positioned N-terminally of the GEMs, wherein the signal sequence comprises or consists of SEQ ID NO 26, and/or
b. comprising one or more linker (leader) sequences positioned N-terminally of and/or between the GEMs, wherein the linker sequence comprises or consists of one or more linker sequences of or from within a sequence according to SEQ ID NO 27-35, and/or
c. comprising or consisting of one or more of SEQ ID NO 27-35.

17. (canceled)

18. (canceled)

19. A combination of multiple recombinant polypeptides, said combination comprising the two to six granulin/epithelin modules (GEMs) according to claim 1, comprising GEM E, and additionally one or more of GEM B, GEM F, GEM C and GEM D, and/or comprising two to six granulin/epithelin modules (GEMs), comprising GEM F, and additionally one or more of GEM B, GEM E, GEM A and GEM D.

20. A nucleic acid molecule encoding the recombinant polypeptide or combination of multiple recombinant polypeptides according to claim 1.

21. The nucleic acid molecule according to claim 20, present as a combination of multiple nucleic acid molecules, each encoding one or more of a combination of recombinant polypeptides.

22. The nucleic acid molecule according to claim 20, in the form of a vector configured to express the recombinant polypeptide after administration to a subject.

23. The nucleic acid molecule according to claim 20, wherein the vector is selected from the group consisting of an adenovirus, adeno-associated virus, lentivirus and baculovirus.

24. The nucleic acid molecule according to claim 20, wherein said molecule encodes multiple GEMs configured for expression as a polycistronic mRNA, wherein said GEMs are encoded by a single nucleic acid molecule and configured for cleavage post-transcription and/or post-translation, and/or wherein the polycistronic mRNA comprises multiple internal ribosome entry sites (IRES), enabling expression of multiple distinct and soluble GEM polypeptides.

25. (canceled)

26. A pharmaceutical composition comprising the recombinant polypeptide or combination according to claim 1, with a pharmaceutically acceptable excipient.

27. A method of treating a neurodegenerative disease in a subject, the method comprising administering a therapeutically effective amount of the polypeptide or combination according to claim 1 to a subject in need thereof, optionally in the form of a-nucleic acid molecule encoding the recombinant polypeptide or combination of multiple recombinant polypeptides according to claim 1 and/or a pharmaceutical composition comprising the recombinant polypeptide or combination according to claim 1.

28. The method according to claim 27, wherein the neurodegenerative disease is selected from the group consisting of Amyotrophic Lateral Sclerosis (ALS), Frontotemporal Dementia (FTD), Spinal Muscular Atrophy (SMA), Alzheimer's Disease (AD) and Parkinson's Disease (PD).

29. The method according to claim 27, wherein the neurodegenerative disease is selected from a disease associated with aberrant lysosomal function.
